SEQUENCE LISTING (24 sequences)

SEQ ID NO: 1 - protein, 19 aa, synthetic construct
  source 1..19: note = N-terminal secretion signal
MGWSCIILFLVATATGVHS

SEQ ID NO: 2 - protein, 5 aa, synthetic construct
  source 1..5: note = Linker
GGGGS

SEQ ID NO: 3 - protein, 5 aa, synthetic construct
  source 1..5: note = Linker; n= any integer
GGGGA

SEQ ID NO: 4 - protein, 5 aa, synthetic construct
  source 1..5: note = Linker n=2
GGGGA

SEQ ID NO: 5 - protein, 5 aa, synthetic construct
  source 1..5: note = enterokinase
DDDDK

SEQ ID NO: 6 - protein, 4 aa, synthetic construct
  source 1..4: note = Factor Xa cleavage site
IEGR

SEQ ID NO: 7 - protein, 5 aa, synthetic construct
  source 1..5: note = Linker; n= any integer
GSGGS

SEQ ID NO: 8 - protein, 4 aa, synthetic construct
  source 1..4: note = Linker; n= any integer
GGGS

SEQ ID NO: 9 - skipped

SEQ ID NO: 10 - DNA, 3528 bp, synthetic construct
  source 1..3528: mol_type = other DNA; note = IgG-CTF-2 format, neprolysin, anti-gD, heavy chain
atgggatggtcatgtatcatcctttttctagtagcaactgcaaccggtgtacattcagaagttcagctggtggagtctggcggtggcctggtgcagccagggggctcactccgtttgtcctgtgccgcttctggctactccatcacctccgactttgcctggaactgggtccgtcaggccccgggtaagggcctggaatgggttggatacattagttactctggaaccactagctataaccctagcctgaagtcccgtatcactataagtcgcgacaattccaaaaacacattctacctgcagatgaacagcctgcgtgctgaggacactgccgtctattattgtgctcgagaaaactactatggccgttctcacgttgggtacttcgacgtctggggtcaaggaaccctggtcaccgtctcgagtgcctccaccaagggcccatcggtcttccccctggcaccctcctccaagagcacctctgggggcacagcggccctgggctgcctggtcaaggactacttccccgagccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggcggcggcggatcctacgacgacggcatctgcaagagcagcgactgcatcaagagcgccgccagactgatccagaacatggacgccaccaccgagccctgcaccgacttcttcaagtacgcctgcggcggctggctgaagagaaacgtgatccccgagaccagcagcagatacggcaacttcgacatcctgagagacgagctggaggtggtgctgaaggatgtgctgcaggagcccaagaccgaggacatcgtggccgtgcagaaggccaaggccctgtacagaagctgcatcaacgagagcgccatcgacagcagaggcggcgagcccctgctgaagctgctgcccgacatctacggctggcccgtggccaccgagaactgggagcagaagtacggcgccagctggaccgccgagaaggccatcgcccagctgaacagcaagtacggcaagaaggtgctgatcaacctgttcgtgggcaccgacgacaagaacagcgtgaaccacgtgatccacatcgaccagcccagactgggcctgcccagcagagactactacgagtgcaccggcatctacaaggaggcctgcaccgcctacgtggacttcatgatcagcgtggccagactgatcagacaggaggagagactgcccatcgacgagaaccagctggccctggagatgaacaaggtgatggagctggagaaggagatcgccaacgccaccgccaagcccgaggacagaaacgaccccatgctgctgtacaacaagatgaccctggcccagatccagaacaacttcagcctggagatcaacggcaagcccttcagctggctgaacttcaccaacgagatcatgagcaccgtgaacatcagcatcaccaacgaggaggatgtggtggtgtacgcccccgagtacctgaccaagctgaagcccatcctgaccaagtacagcgccagagacctgcagaacctgatgagctggagattcatcatggacctggtgagcagcctgagcagaacctacaaggagagcagaaacgccttcagaaaggccctgtacggcaccaccagcgagaccgccacctggagaagatgcgccaactacgtgaacggcaacatggagaacgccgtgggcagactgtacgtggaggccgccttcgccggcgagagcaagcacgtggtggaggacctgatcgcccagatcagagaggtgttcatccagaccctggacgacctgacctggatggacgccgagaccaagaagagagccgaggagaaggccctggccatcaaggagagaatcggctaccccgacgacatcgtgagcaacgacaacaagctgaacaacgagtacctggagctgaactacaaggaggacgagtacttcgagaacatcatccagaacctgaagttcagccagagcaagcagctgaagaagctgagagagaaggtggacaaggacgagtggatcagcggcgccgccgtggtgaacgccttctacagcagcggcagaaaccagatcgtgttccccgccggcatcctgcagccccccttcttcagcgcccagcagagcaacagcctgaactacggcggcatcggcatggtgatcggccacgagatcacccacggcttcgacgacaacggcagaaacttcaacaaggacggcgacctggtggactggtggacccagcagagcgccagcaacttcaaggagcagagccagtgcatggtgtaccagtacggcaacttcagctgggacctggccggcggccagcacctgaacggcatcaacaccctgggcgagaacatcgccgacaacggcggcctgggccaggcctacagagcctaccagaactacatcaagaagaacggcgaggagaagctgctgcccggcctggacctgaaccacaagcagctgttcttcctgaacttcgcccaggtgtggtgcggcacctacagacccgagtacgccgtgaacagcatcaagaccgatgtgcacagccccggcaacttcagaatcatcggcaccctgcagaacagcgccgagttcagcgaggccttccactgcagaaagaacagctacatgaaccccgagaagaagtgcagagtgtggtga

SEQ ID NO: 11 - DNA, 714 bp, synthetic construct
  source 1..714: mol_type = other DNA; note = IgG-CTF-2 format, neprolysin, anti-gD, light chain:
atgggatggtcatgtatcatcctttttctagtagcaactgcaaccggtgtacattcagacatccagatgacccagtctccatcctccctgtctgcatctgtaggagacagagtcaccatcacttgccgggcaagtgcgtctgttgactcttacggtaacagctttatacattggtatcagcagaaaccagggaaagcccctaagctcctgatctatcgtgcatccgatttggaaagtggggtcccatcaaggttcagtggcagtggatctgggacagatttcactctcaccatcagcagtctgcaacctgaagattttgcaacttactactgtcaacagaattacgctgaccctttcacgttcggccaaggtaccaaggtggagatcaaacgaactgtggctgcaccatctgtcttcatcttcccgccatctgatgagcagttgaaatctggaactgcttctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 12 - DNA, 3495 bp, synthetic construct
  source 1..3495: mol_type = other DNA; note = IgG-CTF-2 format, neprolysin, crenezumab, heavy chain:
atgggatggtcatgtatcatcctttttctagtagcaactgcaaccggtgtacattcagaagttcagctggtcgagtctgggggaggcttagtgcagcctggagggtccctgagactctcctgtgcagcctctggattcactttcagtagctatggcatgtcttgggttcgccaggctccaggcaagggtctcgaattggtcgcaagcatcaatagtaatggtggtagcacctattatccagacagtgtgaagggccgattcaccatctccagagacaatgccaagaactccctgtacctgcaaatgaacagtctgagagctgaggacaccgccgtgtattactgtgcaagtggtgactactggggccaaggcaccactgtcacagtctcgagtgcctccaccaagggcccatcggtcttccccctggcaccctcctccaagagcacctctgggggcacagcggccctgggctgcctggtcaaggactacttccccgagccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggcggcggcggatcctacgacgacggcatctgcaagagcagcgactgcatcaagagcgccgccagactgatccagaacatggacgccaccaccgagccctgcaccgacttcttcaagtacgcctgcggcggctggctgaagagaaacgtgatccccgagaccagcagcagatacggcaacttcgacatcctgagagacgagctggaggtggtgctgaaggatgtgctgcaggagcccaagaccgaggacatcgtggccgtgcagaaggccaaggccctgtacagaagctgcatcaacgagagcgccatcgacagcagaggcggcgagcccctgctgaagctgctgcccgacatctacggctggcccgtggccaccgagaactgggagcagaagtacggcgccagctggaccgccgagaaggccatcgcccagctgaacagcaagtacggcaagaaggtgctgatcaacctgttcgtgggcaccgacgacaagaacagcgtgaaccacgtgatccacatcgaccagcccagactgggcctgcccagcagagactactacgagtgcaccggcatctacaaggaggcctgcaccgcctacgtggacttcatgatcagcgtggccagactgatcagacaggaggagagactgcccatcgacgagaaccagctggccctggagatgaacaaggtgatggagctggagaaggagatcgccaacgccaccgccaagcccgaggacagaaacgaccccatgctgctgtacaacaagatgaccctggcccagatccagaacaacttcagcctggagatcaacggcaagcccttcagctggctgaacttcaccaacgagatcatgagcaccgtgaacatcagcatcaccaacgaggaggatgtggtggtgtacgcccccgagtacctgaccaagctgaagcccatcctgaccaagtacagcgccagagacctgcagaacctgatgagctggagattcatcatggacctggtgagcagcctgagcagaacctacaaggagagcagaaacgccttcagaaaggccctgtacggcaccaccagcgagaccgccacctggagaagatgcgccaactacgtgaacggcaacatggagaacgccgtgggcagactgtacgtggaggccgccttcgccggcgagagcaagcacgtggtggaggacctgatcgcccagatcagagaggtgttcatccagaccctggacgacctgacctggatggacgccgagaccaagaagagagccgaggagaaggccctggccatcaaggagagaatcggctaccccgacgacatcgtgagcaacgacaacaagctgaacaacgagtacctggagctgaactacaaggaggacgagtacttcgagaacatcatccagaacctgaagttcagccagagcaagcagctgaagaagctgagagagaaggtggacaaggacgagtggatcagcggcgccgccgtggtgaacgccttctacagcagcggcagaaaccagatcgtgttccccgccggcatcctgcagccccccttcttcagcgcccagcagagcaacagcctgaactacggcggcatcggcatggtgatcggccacgagatcacccacggcttcgacgacaacggcagaaacttcaacaaggacggcgacctggtggactggtggacccagcagagcgccagcaacttcaaggagcagagccagtgcatggtgtaccagtacggcaacttcagctgggacctggccggcggccagcacctgaacggcatcaacaccctgggcgagaacatcgccgacaacggcggcctgggccaggcctacagagcctaccagaactacatcaagaagaacggcgaggagaagctgctgcccggcctggacctgaaccacaagcagctgttcttcctgaacttcgcccaggtgtggtgcggcacctacagacccgagtacgccgtgaacagcatcaagaccgatgtgcacagccccggcaacttcagaatcatcggcaccctgcagaacagcgccgagttcagcgaggccttccactgcagaaagaacagctacatgaaccccgagaagaagtgcagagtgtggtga

SEQ ID NO: 13 - DNA, 717 bp, synthetic construct
  source 1..717: mol_type = other DNA; note = IgG-CTF-2 format, neprolysin, crenezumab, light chain
atgggatggtcatgtatcatcctttttctagtagcaactgcaaccggtgtacattcagatatcgtgatgacccaatctccactctccctgcctgtcactcctggtgagcctgcctccatctcttgcagatctagtcagagccttgtatatagtaatggagacacctatttacattggtatctgcagaagccaggccagtctccacagctcctgatctacaaagtttccaaccgattttctggggtcccagacaggttcagtggcagtggatcagggacagatttcacactcaagatcagcagagtggaggctgaggatgtgggagtttattactgctctcaaagtacacatgttccttggacgttcggccaaggtaccaaggtggagatcaaacgaactgtggctgcaccatctgtcttcatcttcccgccatctgatgagcagttgaaatctggaactgcttctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 14 - DNA, 3495 bp, synthetic construct
  source 1..3495: mol_type = other DNA; note = IgG-CTF-2 format, neprolysin, solanezumab, heavy chain
atgggatggtcatgtatcatcctttttctagtagcaactgcaaccggtgtacattcagaagtgcagctggtggagtctgggggaggcttggtacagcctggggggtccctgagactctcctgtgcagcctctggattcacctttagcagatattccatgagctgggtccgccaggctccagggaaggggctggagctggtcgcacaaattaatagcgttggtaatagcacatactatccagacaccgtgaagggccggttcaccatctccagagataatgccaagaacacgctgtatctgcaaatgaacagcctgagagccgaggacacggccgtatattactgtgcgtcaggtgactactggggccaaggaaccctggtcaccgtctcgagtgcctccaccaagggcccatcggtcttccccctggcaccctcctccaagagcacctctgggggcacagcggccctgggctgcctggtcaaggactacttccccgagccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtggcggcggcggatcctacgacgacggcatctgcaagagcagcgactgcatcaagagcgccgccagactgatccagaacatggacgccaccaccgagccctgcaccgacttcttcaagtacgcctgcggcggctggctgaagagaaacgtgatccccgagaccagcagcagatacggcaacttcgacatcctgagagacgagctggaggtggtgctgaaggatgtgctgcaggagcccaagaccgaggacatcgtggccgtgcagaaggccaaggccctgtacagaagctgcatcaacgagagcgccatcgacagcagaggcggcgagcccctgctgaagctgctgcccgacatctacggctggcccgtggccaccgagaactgggagcagaagtacggcgccagctggaccgccgagaaggccatcgcccagctgaacagcaagtacggcaagaaggtgctgatcaacctgttcgtgggcaccgacgacaagaacagcgtgaaccacgtgatccacatcgaccagcccagactgggcctgcccagcagagactactacgagtgcaccggcatctacaaggaggcctgcaccgcctacgtggacttcatgatcagcgtggccagactgatcagacaggaggagagactgcccatcgacgagaaccagctggccctggagatgaacaaggtgatggagctggagaaggagatcgccaacgccaccgccaagcccgaggacagaaacgaccccatgctgctgtacaacaagatgaccctggcccagatccagaacaacttcagcctggagatcaacggcaagcccttcagctggctgaacttcaccaacgagatcatgagcaccgtgaacatcagcatcaccaacgaggaggatgtggtggtgtacgcccccgagtacctgaccaagctgaagcccatcctgaccaagtacagcgccagagacctgcagaacctgatgagctggagattcatcatggacctggtgagcagcctgagcagaacctacaaggagagcagaaacgccttcagaaaggccctgtacggcaccaccagcgagaccgccacctggagaagatgcgccaactacgtgaacggcaacatggagaacgccgtgggcagactgtacgtggaggccgccttcgccggcgagagcaagcacgtggtggaggacctgatcgcccagatcagagaggtgttcatccagaccctggacgacctgacctggatggacgccgagaccaagaagagagccgaggagaaggccctggccatcaaggagagaatcggctaccccgacgacatcgtgagcaacgacaacaagctgaacaacgagtacctggagctgaactacaaggaggacgagtacttcgagaacatcatccagaacctgaagttcagccagagcaagcagctgaagaagctgagagagaaggtggacaaggacgagtggatcagcggcgccgccgtggtgaacgccttctacagcagcggcagaaaccagatcgtgttccccgccggcatcctgcagccccccttcttcagcgcccagcagagcaacagcctgaactacggcggcatcggcatggtgatcggccacgagatcacccacggcttcgacgacaacggcagaaacttcaacaaggacggcgacctggtggactggtggacccagcagagcgccagcaacttcaaggagcagagccagtgcatggtgtaccagtacggcaacttcagctgggacctggccggcggccagcacctgaacggcatcaacaccctgggcgagaacatcgccgacaacggcggcctgggccaggcctacagagcctaccagaactacatcaagaagaacggcgaggagaagctgctgcccggcctggacctgaaccacaagcagctgttcttcctgaacttcgcccaggtgtggtgcggcacctacagacccgagtacgccgtgaacagcatcaagaccgatgtgcacagccccggcaacttcagaatcatcggcaccctgcagaacagcgccgagttcagcgaggccttccactgcagaaagaacagctacatgaaccccgagaagaagtgcagagtgtggtga

SEQ ID NO: 15 - DNA, 717 bp, synthetic construct
  source 1..717: mol_type = other DNA; note = IgG-CTF-2 format, neprolysin, solanezumab, light chain
atgggatggtcatgtatcatcctttttctagtagcaactgcaaccggtgtacattcagatgttgtgatgactcaatctccactctccctgcccgtcacccttggacagccagcctccatctcatgcaggagtagtcaaagcctcatatacagtgatggaaacgcctacttgcattggtttctccagaagccaggccaatctccaaggctcctaatttataaggtttctaacagattctctggcgtcccagacagattcagcggcagtgggtcaggcactgatttcacactgaaaatcagcagggtggaggctgaggatgttggcgtgtattactgctcgcaatctactcacgttccttggacgttcggccaaggtaccaaggtggagatcaaacgaactgtggctgcaccatctgtcttcatcttcccgccatctgatgagcagttgaaatctggaactgcttctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 16 - DNA, 1416 bp, synthetic construct
  source 1..1416: mol_type = other DNA; note = IgG-NTF(LC)-2 format, MMP3-D4K-4, anti-gD, heavy chain:
atgggatggtcatgtatcatcctttttctagtagcaactgcaaccggtgtacattcagaggttcagctggtggagtctggcggtggcctggtgcagccagggggctcactccgtttgtcctgtgccgcttctggctactccatcacctccgactttgcctggaactgggtccgtcaggccccgggtaagggcctggaatgggttggatacattagttactctggaaccactagctataaccctagcctgaagtcccgtatcactataagtcgcgacaattccaaaaacacattctacctgcagatgaacagcctgcgtgctgaggacactgccgtctattattgtgctcgggaaaactactatggccgttctcacgttgggtacttcgacgtctggggtcaaggaaccctggtcaccgtctcgagtgcctccaccaagggcccatcggtcttccccctggcaccctcctccaagagcacctctgggggcacagcggccctgggctgcctggtcaaggactacttccccgagccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgtgacaaaactcacacatgcccaccgtgcggcggaggcggagccggcgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggagcccccatcgagaaaaccatctccaaagccaaagggcagccccgagaaccacaggtgtacaccctgcccccatccgaggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttga

SEQ ID NO: 17 - DNA, 2124 bp, synthetic construct
  source 1..2124: mol_type = other DNA; note = IgG-NTF(LC)-2 format, MMP3-D4K-4, anti-gD, light chain
atgggatggtcatgtatcatcctttttctagtagcaactgcaaccggtgtacattcataccccctggacggcgccgctagaggcgaggacaccagcatgaacctggtgcagaagtacctggagaactactatgacctgaagaaggacgatgacgataaggtgagaaggaaggacagcggccccgtggtcaagaagatcagagagatgcagaagttcctgggcctggaggtgaccggcaagctggacagcgacaccctggaggtgatgagaaagcccagatgcggcgtgcccgacgtgggccacttcagaaccttccccggcatccccaagtggagaaagacccacctgacctacagaatcgtgaactacacccccgacctgcccaaggacgccgtggacagcgccgtggagaaggccctgaaggtgtgggaggaagtgacccccctgaccttcagcagactgtacgagggcgaggccgacatcatgatcagcttcgccgtgagagagcacggcgacttctaccccttcgacggccccggcaacgtgctggcccacgcctacgcccccggccccggcatcaacggcgacgcccacttcgacgatgacgagcagtggaccaaggacaccacaggcaccaacctgttcctggtggccgctcacgagatcggccacagcctgggcctgttccacagcgccaacaccgaggccctgatgtaccccctgtaccacagcctgaccgacctgaccagattcagactgagccaggacgatatcaacggcatccagagcctgtacggccctccacctgacagccccgagacccccctggtgcccaccgagcccgtgcctccagagcccggcacccccgccaactgcgaccccgccctgagcttcgacgccgtgagcaccctgagaggcgagatcctgatcttcaaggacagacacttctggagaaagagcctgagaaagctggagcccgagctgcacctgatcagctccttctggcccagcctgcccagcggcgtggacgccgcttacgaggtgaccagcaaggacctggtgttcatcttcaagggcaaccagttctgggccatcagaggcaacgaggtgagagccggctaccccagaggcatccacaccctgggcttccctccaaccgtgagaaagatcgacgccgctatcagcgacaaggagaagaacaagacctacttcttcgtggaggacaagtactggagattcgacgagaagagaaacagcatggagcccggcttccccaagcagatcgccgaggacttccccggcatcgacagcaagatcgacgccgtgttcgaggagttcggcttcttctacttcttcaccggcagctcccagctggagttcgaccccaacgccaagaaggtgacccacaccctgaagagcaacagctggctgaactgcggcggaggcggagccggcggaggcggagccgacatccagatgacccagtctccatcctccctgtctgcatctgtaggagacagagtcaccatcacttgccgggcaagtgcgtctgttgactcttacggtaacagctttatacattggtatcagcagaaaccagggaaagcccctaagctcctgatctatcgtgcatccgatttggaaagtggggtcccatcaaggttcagtggcagtggatctgggacagatttcactctcaccatcagcagtctgcaacctgaagattttgcaacttactactgtcaacagaattacgctgaccctttcacgttcggccaaggtaccaaggtggagatcaaacgaactgtggctgcaccatctgtcttcatcttcccgccatctgatgagcagttgaaatctggaactgcttctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgttaa

SEQ ID NO: 18 - DNA, 1416 bp, synthetic construct
  source 1..1416: mol_type = other DNA; note = IgG-NTF(LC)-2 format, MMP3-D4K-4, RF61 wild-type, heavy chain
atgggatggtcatgtatcatcctttttctagtagcaactgcaaccggtgtacattcacagctgcagctgcaggagagcggccccggcctggtgaagcccagcgagaccctgagcctgacctgcaccgtgagcggcggcagcatcagcagaggcagccactactggggctggatcagacagccccccggcaagggcctggagtggatcggcagcatctactacagcggcaacacctacttcaaccccagcctgaagagcagagtgaccatcagcgtggacaccagcaagaaccagttcagcctgaagctgagcagcgtgaccgccgccgacaccgccgtgtactactgcgccagactgggccccgacgactacaccctggacggcatggacgtgtggggccagggcaccaccgtgaccgtctcgagtgcctccaccaagggcccatcggtcttccccctggcaccctcctccaagagcacctctgggggcacagcggccctgggctgcctggtcaaggactacttccccgagccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgtgacaaaactcacacatgcccaccgtgcggcggaggcggagccggcgggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcggagcccccatcgagaaaaccatctccaaagccaaagggcagccccgagaaccacaggtgtacaccctgcccccatccgaggaagagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggttga

SEQ ID NO: 19 - DNA, 2118 bp, synthetic construct
  source 1..2118: mol_type = other DNA; note = IgG-NTF(LC)-2 format, MMP3-D4K-4, RF61 wild-type, light chain
atgggatggtcatgtatcatcctttttctagtagcaactgcaaccggtgtacattcataccccctggacggcgccgctagaggcgaggacaccagcatgaacctggtgcagaagtacctggagaactactatgacctgaagaaggacgatgacgataaggtgagaaggaaggacagcggccccgtggtcaagaagatcagagagatgcagaagttcctgggcctggaggtgaccggcaagctggacagcgacaccctggaggtgatgagaaagcccagatgcggcgtgcccgacgtgggccacttcagaaccttccccggcatccccaagtggagaaagacccacctgacctacagaatcgtgaactacacccccgacctgcccaaggacgccgtggacagcgccgtggagaaggccctgaaggtgtgggaggaagtgacccccctgaccttcagcagactgtacgagggcgaggccgacatcatgatcagcttcgccgtgagagagcacggcgacttctaccccttcgacggccccggcaacgtgctggcccacgcctacgcccccggccccggcatcaacggcgacgcccacttcgacgatgacgagcagtggaccaaggacaccacaggcaccaacctgttcctggtggccgctcacgagatcggccacagcctgggcctgttccacagcgccaacaccgaggccctgatgtaccccctgtaccacagcctgaccgacctgaccagattcagactgagccaggacgatatcaacggcatccagagcctgtacggccctccacctgacagccccgagacccccctggtgcccaccgagcccgtgcctccagagcccggcacccccgccaactgcgaccccgccctgagcttcgacgccgtgagcaccctgagaggcgagatcctgatcttcaaggacagacacttctggagaaagagcctgagaaagctggagcccgagctgcacctgatcagctccttctggcccagcctgcccagcggcgtggacgccgcttacgaggtgaccagcaaggacctggtgttcatcttcaagggcaaccagttctgggccatcagaggcaacgaggtgagagccggctaccccagaggcatccacaccctgggcttccctccaaccgtgagaaagatcgacgccgctatcagcgacaaggagaagaacaagacctacttcttcgtggaggacaagtactggagattcgacgagaagagaaacagcatggagcccggcttccccaagcagatcgccgaggacttccccggcatcgacagcaagatcgacgccgtgttcgaggagttcggcttcttctacttcttcaccggcagctcccagctggagttcgaccccaacgccaagaaggtgacccacaccctgaagagcaacagctggctgaactgcggcggaggcggagccggcggaggcggagcccagagcgtgctgacccagccccccagcgccagcggcacccccggccagagagtgaccatcagctgcagcggcagcagcagcaacatcggcagcaactacgtgtactggtatcagcagctgcccggcaccgcccccaagctgctgatctacagaaacaaccagagacccagcggcgtgcccgacagattcagcggcagcaagagcggcaccagcgccagcctggccatcagcggcctgagaagcgaggacgaggccgactactactgcgccacctgggacgacagcctgagcgccgtgatcttcggcggcggtaccaagctgaccgtccttggccaacctaaggctgcaccatctgtcaccctcttcccgccatcttctgaggagttgcaagctaacaaagccactcttgtgtgcctgatcagtgacttctatcccggagcggtcacagtagcgtggaaggcggatagctcccccgtaaaggctggcgtcgagacgactaccccttcgaagcagagcaacaacaaatacgccgccagcagctacctgtcgctgaccccagaacagtggaagagccacaaaagctactcctgccaagtcacccatgagggctcgaccgtcgaaaagaccgtcgccccgacagagtgttcttga

SEQ ID NO: 20 - DNA, 777 bp, synthetic construct
  source 1..777: mol_type = other DNA; note = Fab-NTF(LC)-1 format, MMP3-D4K-4, RF61 wild-type, heavy chain
atgggatggtcatgtatcatcctttttctagtagcaactgcaaccggtgtacattcacagctgcagctgcaggagagcggccccggcctggtgaagcccagcgagaccctgagcctgacctgcaccgtgagcggcggcagcatcagcagaggcagccactactggggctggatcagacagccccccggcaagggcctggagtggatcggcagcatctactacagcggcaacacctacttcaaccccagcctgaagagcagagtgaccatcagcgtggacaccagcaagaaccagttcagcctgaagctgagcagcgtgaccgccgccgacaccgccgtgtactactgcgccagactgggccccgacgactacaccctggacggcatggacgtgtggggccagggcaccaccgtgaccgtctcgagtgcctccaccaagggcccatcggtcttccccctggcaccctcctccaagagcacctctgggggcacagcggccctgggctgcctggtcaaggactacttccccgagccggtgacggtgtcgtggaactcaggcgccctgaccagcggcgtgcacaccttcccggctgtcctacagtcctcaggactctactccctcagcagcgtggtgactgtgccctctagcagcttgggcacccagacctacatctgcaacgtgaatcacaagcccagcaacaccaaggtggacaagaaagttgagcccaaatcttgtgacaaaactcacacagattataaggacgatgacgataaatga

SEQ ID NO: 21 - DNA, 2118 bp, synthetic construct; identical to SEQ ID NO: 19
  source 1..2118: mol_type = other DNA; note = Fab-NTF(LC)-1 format, MMP3-D4K-4, RF61 wild-type, light chain

SEQ ID NO: 22 - protein, 22 aa, synthetic construct
  source 1..22: note = IgG hinge sequence
SCDKTHTCPPCPAPELLGGPSV

SEQ ID NO: 23 - protein, 15 aa, synthetic construct
  source 1..15: note = IgG WT
CPPCPAPELLGGPSV

SEQ ID NO: 24 - protein, 15 aa, synthetic construct
  source 1..15: note = IgG1
CPPCGGGGAGGGGAV

(19) United States
(12) Patent Application Publication
(10) Pub. No.: US 2026/0183373 A1
(43) Pub. Date: Jul. 2, 2026

(54) FUSIONS WITH PROTEASES AND USES THEREOF

(71) Applicant: Genentech, Inc., South San Francisco, CA (US)

(72) Inventors: Gregory Alan LAZAR, Pacifica, CA (US); Brandon LEONARD, Pacifica, CA (US); Matthew G. ROMEI, Richmond, CA (US)

(73) Assignee: Genentech, Inc., South San Francisco, CA (US)

(21) Appl. No.: 19/321,561

(22) Filed: Sep. 8, 2025

Related U.S. Application Data
(63) Continuation of application No. PCT/US2024/019042, filed on Mar. 8, 2024.
(60) Provisional application No. 63/451,509, filed on Mar. 10, 2023.

(51) Int. Cl. A61K 38/48; A61K 39/395; C07K 16/30; C12N 9/64

(52) U.S. Cl. CPC A61K 38/4886; A61K 39/3955; C07K 16/30; C12N 9/6491; C12Y 304/24017; C07K 2317/52; C07K 2317/55; C07K 2317/92; C07K 2319/01

(57) ABSTRACT

Provided herein are fusion proteins that involve an antibody that binds to a target fused to a protease that cleaves a substrate, and the target is in the proximity of the substrate. The target and the substrate in some cases are the same molecule. Methods of making and using such fusion proteins are also provided.

### CROSS-REFERENCE TO RELATED APPLICATIONS

This application is a Continuation of International Application No. PCT/US2024/019042, filed Mar. 8, 2024, which claims benefit of priority to U.S. Application No. 63/451,509, filed Mar. 10, 2023, each of which is incorporated herein by reference in its entirety.

### SEQUENCE LISTING

This application contains a Sequence Listing which has been submitted electronically in XML format and is hereby incorporated by reference in its entirety. Said XML copy, created on Feb. 16, 2026, is named P38275-US-1_SL.xml and is 48,281 bytes in size.

### FIELD

The present disclosure discloses fusion proteins comprising an antibody fused to a protease that cleaves a substrate, when the antibody binds to a target and the target is in the proximity of the substrate. In addition, the present disclosure also provides polynucleotides encoding the disclosed fusion proteins, and vectors and host cells comprising such polynucleotides. The present disclosure further provides methods for producing the fusion proteins, pharmaceutical compositions comprising the same, and uses thereof.

### BACKGROUND

Protein based therapeutics are a successful class of drugs that address major medical needs in a variety of therapeutic areas. For example, monoclonal antibodies are an immensely successful class of drugs that address major medical needs in a variety of therapeutic areas. The success of antibodies stems in part from their high specificity, capability for immune recruitment, long serum half-life, relatively low immunogenicity, and streamlined discovery methods. Despite these features, an inherent limitation of antibodies is their general reliance on stoichiometric target binding to induce the desired therapeutic effect. This aspect of antibodies can impede their effective application to some targets of therapeutic interest, specifically those of high abundance and those for which there are barriers to site of action. Other factors that have also hindered clinical success has been poor exposure of systemically administered antibody-based drugs. For example, low exposure of systemic antibodies to the CNS (˜0.1%) (Wang et al., 2018, Fluids Barriers Cns. 15, 10; Lemere, 2013, Mol. Neurodegener. 8, 36-36; Yu et al., 2011, Sci Transl Med. 3, 84ra44) has demanded extraordinarily high doses of antibodies targeting pathogenic amyloid-β and tau proteins within the central nervous system, and may be a factor that has hindered their clinical success (Lemere, 2013, Mol. Neurodegener. 8, 36-36; Kwan et al., 2021, Dement Geriatr Cogn. 49, 334-348; Dyck 2018, Biol Psychiat. 83, 311-319).

Enzymes are another class of proteins with a sub-stoichiometric mechanism of action. In contrast to antibodies, a single enzyme can react with many substrate molecules with a high catalytic rate and turnover, thus enabling low doses to maintain sufficient activity. Enzymes have been approved for the treatment of cancer, blood disorders, lysosomal storage disorders, and metabolic deficiencies, among many other conditions (Cioni et al., 2021 Curr Med Chem.; Tandon et al., 2021 J. Drug Deliv Sci Tec.; Fuente et al., 2021 Int J. Mol Sci. 22, 9181). However, several drawbacks limit more widespread application of this therapeutic class including short half-life, lack of tissue specificity, broad substrate specificity, and high immunogenicity when not of human origin.

Previous work has combined antibodies with enzymes to circumvent the drawbacks of antibody and enzyme based therapeutics. However, these studies focused on the former's use as a delivery vehicle, with varied preclinical and limited clinical success (Zhou et al., 2019 Trends Mol. Med. 25, 1094-1109). Antibody delivery of enzymes has been studied for three main applications. The first involves replacing the function of inactive native enzymes, termed enzyme replacement therapy, and has demonstrated clinical success for antibody-mediated delivery of enzymatic activity to the lysosome, cytosol, and brain (Zhou et al., 2019 Trends Mol. Med. 25, 1094-1109; Silver et al. 2021, Trends Pharmacol Sci. 42, 1064-1081; Yi et al., J. Mol Med. 95, 513-521; Giugliani et al. 2018, Orphanet J. Rare Dis. 13, 110). The second, referred to as antibody-directed enzyme prodrug therapy (ADEPT), combines a tumor-targeting antibody-enzyme fusion with a systemically-delivered inactive prodrug. Enzymatic activation of the prodrug locally at the tumor site is intended to minimize toxicity. Most ADEPT development has been preclinical, with minimal success in early clinical studies (Zhou et al., 2019 Trends Mol. Med. 25, 1094-1109; Silver et al. 2021, Trends Pharmacol Sci. 42, 1064-1081; Sharma et al. 2017 Adv Drug Deliver Rev. 118, 2-7; Mayer et al. 2006 Clin Cancer Res. 12, 6509-6516). The third is a broad category that includes any attempt to localize or direct enzymes to specific tissues, cell types, or subcellular locations. For example, antibodies that bind the human insulin receptor or transferrin receptor have been used to shuttle cargo, including enzymes, across the blood brain barrier to treat enzyme deficiencies (Zhou et al., 2019 Trends Mol. Med. 25, 1094-1109; Boado et al. 2013 Bioconjugate Chem. 24, 1741-1749). Cancer therapies have been explored that combine a tumor-targeted antibody with cytotoxic enzymes, such as RNases and various proapoptotic enzymes (D'Avino et al. 2014 Protein Eng Des Sel. 27, 83-88; Xu et al 2004 J. Immunol. 173, 61-67; Andrady et al. 2011 Immunotherapy 3, 193-211). Therefore, there is an unmet need in providing new therapeutic approaches for neutralization of difficult therapeutic targets.

All references cited herein, including patent applications, patent publications, and UniProtKB/Swiss-Prot Accession numbers are herein incorporated by reference in their entirety, as if each individual reference were specifically and individually indicated to be incorporated by reference.

### BRIEF SUMMARY

It is to be understood that one, some, or all of the properties of the various embodiments described herein may be combined to form other embodiments of the present disclosure. These and other aspects of the disclosure will become apparent to one of skill in the art. These and other embodiments of the disclosure are further described by the detailed description that follows.

The present application in one aspect provides fusion proteins comprising an antibody that binds to a target, wherein the antibody is fused to a protease that cleaves a substrate, wherein the target is in the proximity of the substrate. In some embodiments, the protease is fused to one or more polypeptide chains of the antibody. In some embodiments, the protease is fused to the N-terminus and/or C-terminus of one or more polypeptide chains of the antibody.

In some embodiments according to any of the fusion proteins described above, the antibody comprises:
a) a heavy chain variable domain (VH) and a light chain variable domain (VL), wherein the protease is fused to the N-terminus or C-terminus of the VH or VL; or
b) a full-length antibody comprising two heavy chains and two light chains, wherein the protease is fused to the N-terminus or C-terminus of one or both of the two heavy chains and/or the two light chains.

In some embodiments according to any of the fusion proteins described above, the antibody comprises a) a first polypeptide comprising a heavy chain variable (VH) domain and a first heavy chain constant (CH1) domain and b) a second polypeptide comprising a light chain variable (VL) domain and a light chain constant (CL) domain, wherein the protease is fused to the N-terminus of the VL domain, wherein the VH domain and VL domain form a binding domain for the target, wherein a disulfide bond is formed between the CH1 domain and the CL domain. In some embodiments, the antibody does not comprise a Fc fragment.

In some embodiments, the antibody comprises a) a first polypeptide comprising a light chain variable (VL) domain and a light chain constant (CL) domain, b) a second polypeptide comprising a heavy chain variable (VH) domain and first heavy chain constant (CH1) domains, and c) a third polypeptide comprising a Fc region, wherein the protease is fused to the N-terminus of the third polypeptide, and wherein the VH domain and the VL domain form a binding domain for the target.

In some embodiments, the antibody comprises two heavy chains and two light chains, wherein each heavy chain comprises a heavy chain variable (VH) domain and a heavy chain constant (CH) domain, wherein each light chain comprises a light chain variable (VL) domain and a light chain constant (CL) domain, wherein the protease is fused to the N-terminus of the two light chains.

In some embodiments, the antibody comprises two heavy chains and two light chains, wherein each heavy chain comprises a heavy chain variable (VH) domain and a heavy chain constant (CH) domain, wherein each light chain comprises a light chain variable (VL) domain and a light chain constant (CL) domain, wherein the protease is fused to the N-terminus of the two heavy chains.

In some embodiments, the antibody comprises two heavy chains and two light chains, wherein each heavy chain comprises a heavy chain variable (VH) domain and a heavy chain constant (CH) domain, wherein each light chain comprises a light chain variable (VL) domain and a light chain constant (CL) domain, wherein the protease is fused to the N-terminus of the two light chains and N-terminus of the two heavy chains.

In some embodiments, the antibody comprises two heavy chains and two light chains, wherein each heavy chain comprises a heavy chain variable (VH) domain and a heavy chain constant (CH) domain, wherein each light chain comprises a light chain variable (VL) domain and a light chain constant (CL) domain, wherein the protease is fused to the C-terminus of one of the heavy chains.

In some embodiments, the antibody comprises two heavy chains and two light chains, wherein each heavy chain comprises a heavy chain variable (VH) domain and a heavy chain constant (CH) domain, wherein each light chain comprises a light chain variable (VL) domain and a light chain constant (CL) domain, wherein the protease is fused to the C-terminus of both of the heavy chains.

In some embodiments according to any of the fusion proteins described above, the protease is fused with the antibody via a linker. In some embodiments, the linker is a peptide linker, optionally wherein the linker has a length of at least about 5, 10, 15, 20, 25, or 30 amino acids. In some embodiments, the linker is cleavable by a protease that is distinct from the protease comprised in the fusion protein. In some embodiments, the linker is not cleavable.

In some embodiments according to any of the fusion proteins described above, the dissociation rate constant (k_{off}) of antibody to the target is no more than about 5×10⁻³, 1×10⁻³, 5×10⁻⁴, or 1×10⁻⁴.

In some embodiments according to any of the fusion proteins described above, the equilibrium dissociation constant (k_{D}) of the antibody to the target has a range of about 0.1 nM to about 1000 nM.

In some embodiments according to any of the fusion proteins described above, the target and the substrate are expressed in the central nervous system (CNS).

In some embodiments according to any of the fusion proteins described above, the target and the substrate are the same molecule.

In some embodiments according to any of the fusion proteins described above, the protease is a metalloprotease.

In some embodiments according to any of the fusion proteins described above, the target and the substrate are amyloid-β (Aβ). In some embodiments, the antibody and/or the protease targets both Aβ₁₋₄₀ and Aβ₁₋₄₂. In some embodiments, the antibody is crenezumab or a variant thereof, or solanezumab or a variant thereof, optionally wherein the variant of crenezumb comprises G33S on its heavy chains, and further optionally wherein the variant of crenezumb comprises S56F on its light chains. In some embodiments, the protease is selected from the group consisting of neprolysin (NEP), neprolysin-2 (NEP2), endothelin-converting enzyme 1 and 2 (ECE1 and ECE2), angiotensin-converting enzyme (ACE), insulin-degrading enzyme (IDE), matrix metalloproteinase 2 and 9 (MMP2 and MMP9), and matriptase (MTSP1). In some embodiments, the protease is neprolysin. In some embodiments, the antibody comprises two heavy chains and two light chains, wherein the protease is fused to the C-terminus of one or both heavy chains of the antibody.

In some embodiments according to any of the fusion proteins described above, the target and the substrate are an IgG. In some embodiments, the protease is selected from the group consisting of matrix metalloproteinase 3 (MMP3), MMP7, Cathepsin G or a variant thereof. In some embodiments, the protease is MMP3 or a variant thereof. In some embodiments, the antibody comprises a full-length antibody. In some embodiments, the antibody comprises a R335E mutation in the Fc Fragment. In some embodiments, the full-length antibody comprises a hinge region resistant to the protease, optionally wherein the hinge region comprises a (G₄A)₂ sequence. In some embodiments, the antibody comprises a Fab fragment. In some embodiments, the antibody comprises a Rheumatoid factor or a variant thereof.

The present application in another aspect provides isolated nucleic acids encoding any of the fusion proteins described above.

The present application in another aspect provides host cells comprising any of the nucleic acids described above.

The present application in another aspect provides methods of producing any of the fusion proteins described above or a fragment thereof comprising culturing the host cell described above under conditions suitable for the expression of the fusion protein or a fragment thereof. In some embodiments, the method further comprises recovering the fusion protein or a fragment thereof from the host cell.

The present application in another aspect provides fusion proteins produced by the method described above.

The present application in another aspect provides pharmaceutical compositions comprising any of the fusion proteins and a pharmaceutically capable carrier.

The present application in another aspect provides any of the fusion proteins or any of the pharmaceutical compositions described above for use as a medicament.

The present application in another aspect provides any of the fusion proteins or any of the pharmaceutical compositions described above for use in treating a disease or condition.

The present application in another aspect provides uses of any of the fusion proteins or any of the pharmaceutical compositions in the manufacture of a medicament for treating a disease or condition.

The present application in another aspect provides methods of treating an individual having a disease or condition comprising administering to the individual an effective amount of any of the fusion proteins or any of the pharmaceutical compositions.

### BRIEF DESCRIPTION OF THE DRAWINGS

FIG. 1A shows a schematic overview of the design of antibody-guided proteases.

FIG. 1B shows schematics and naming convention of the non-targeted and targeted formats for the antibody-enzyme fusion proteins. The first label refers to the molecule format (Fc: Fragment crystallizable, Fab: Fragment antigen binding, scIgG: monovalent single-chain Immunoglobulin G, IgG: Immunoglobulin G). Enz refers to the free enzyme. The center label describes the fusion format as either NTF (N-terminal fusion) or CTF (C-terminal fusion). Further clarification of the fusion domain is specified within the parentheses. The third and final label denotes the number of proteases per molecule as either 1 or 2.

FIG. 2A shows the results of the activities of an Aβ protease panel on control substrates. The activity of 9 proteases previously shown to cleave Aβ was tested on one of three fluorescence resonance energy transfer (FRET) control substrates. Cleavage of the substrate resulted in increased fluorescence and reported as fold change over no protease. The catalog name of each substrate is indicated within each box.

FIG. 2B shows an in vitro protease screening assay for cleavage of Aβ(1-40) (circles) and Aβ(1-42) (squares).

FIG. 2C shows a chart of the expression yields of various Fc and IgG protease fusion formats. 4 different proteases were expressed in the context of the 8 formats shown in the icons. IgG fusions contained crenezumab variable regions of the anti-Aβ antibody crenezumab, and all heavy chain constant regions were human IgG1. The bar graph shows the expression yields from duplicate 30 ml 293 expressions of each construct.

FIG. 2D shows the size exclusion chromatography traces of purified crenezumab NEP protease fusion formats. Each NEP fusion format was expressed in HEK 293 cells and initially purified using a protein A resin. Size exclusion chromatography (SEC) coupled with sample fractionation was used for further purification. SEC chromatograms revealed the presence of multiple species with each sample containing 2-3 peaks.

FIG. 2E shows the results of the cleavage activity of the central fraction. The central fraction associated with each peak in the chromatograms above was tested for activity by assaying for Aβ(1-40) cleavage.

FIG. 3 shows the comparison of non-targeted and targeted proteolytic degradation of Aβ in different fusion formats. All targeted formats contain the variable domain of the anti-Aβ antibody crenezumab. Formats of the same enzyme and Fab valency and enzyme fusion site are compared in each plot along with enzyme alone. The table shows the EC50 values and visually depicts the targeted and non-targeted formats.

FIG. 4 summarizes the kinetic parameters and EC50 values for the anti-Aβ antibody off-rate series (panel A), shows the results of an in vitro cleavage assay with Aβ(1-40) as substrate (panel B), and shows a graph depicting the relationship between the EC50 value for Aβ(1-40) cleavage and the off-rate for the anti-Aβ targeting antibody (panel C).

FIG. 5A shows a schematic of human and bacterial protease cleavage sites within the hinge of human IgG.

FIG. 5B shows a structural representation of MMP3 (PDB ID: 1SLM(63)). The pro-domain (Pro-D) and catalytic domain (Catalytic) are shown in cartoon representation, while the signaling peptide (SP) and the hemopexin domain (Hemopexin) are not present in the published crystal structure. The four substitution sites for the factor Xa (IEGR) and enterokinase (DDDDK) cleavage sequences are boxed and listed with the corresponding MMP3 residues.

FIG. 5C shows a SDS-PAGE gel depicting the 8 protease cleavage site insertion variants before and after activation with their respective external protease (factor Xa and enterokinase).

FIG. 5D shows the results of a cleavage assay. MMP3-D4K-4, which represents the variant with an enterokinase cleavage site substituted within position 4 of the MMP3 pro-domain. This variant efficiently cleaves a fluorescent MMP3 peptide substrate after the pro-domain is removed with enterokinase (filled circles), while the intact form containing the pro-domain minimally cleaves the substrate (open circles).

FIG. 5E shows a SDS-PAGE gel showing the cleavage of human IgG by MMP3-D4K-4 either with or without the pro-domain at different relative concentrations (10% and 1% w/w) after 24 hours at 37° C. Presence or absence of a component in the reaction is represented by + and −, respectively. MMP3-D4K-4 cleaves the lower hinge of intact human IgG in a sequential manner, first producing a single cleavage product (SCP, in which half of the Fc is lost upon denaturation), then producing F(ab′)₂ and Fc (not shown) after the second cleavage. Enterokinase does not detectably cleave human IgG (lane 3).

FIG. 6A shows a structural representation of two RF61 Fabs (LC: blue, HC: orange) bound to human Fc (red) (PDB ID: 2J6E). The inset highlights residues identified through saturation mutagenesis to be important for Fc binding.

FIG. 6B shows a waterfall plot summarizing the affinities of RF61 variants to human IgG1 over three rounds of saturation mutagenesis and screening. The affinity of wild-type RF61 could not be determined and was estimated as >1 μM. Affinities were measured via SPR on RF61 variants in a mouse IgG2a chimeric format against human IgG1 Fc (see Methods).

FIG. 6C shows quantification of binding between 8 high affinity RF61 variants identified in round 3 of affinity maturation and 38 binding-ablation variants of human Fc. See Example 4 for specific RF61 variants. Late analyte binding values from SPR sensograms representing binding of each RF61 variant to human Fc were used to evaluate the binding-ablation variants, with complete binding ablation shown in white and high binding levels shown in gray. The asterisk denotes that the Fc variant (R355E, variant 12) used in all RF61 IgG constructs to eliminate binding to self.

FIG. 6D shows that mutation of the lower hinge sequence and N-terminal region of CH2 of human IgG1 effectively inhibits cleavage by MMP3-D4K-4. SDS-PAGE gel image (top left) and densitometric representation (top right) confirm resistance of the IgG1 hinge variant to proteolytic cleavage.

FIG. 7A shows cartoon representations of the IgG-MMP3-D4K-4 and the Fab-MMP3-D4K-4 fusion protein formats.

FIG. 7B shows a summary of affinity and relevant mutations for the anti-IgG antibody affinity series tested in FIGS. 7C-7F.

FIG. 7C shows the results of an In vitro cleavage assays measuring proteolytic activity of an anti-IgG affinity series of Fab-MMP3-D4K-4 fusion proteins against a fluorogenic MMP3 peptide substrate. Cleavage of the substrates generated fluorescent signal through dequenching of fluorophores.

FIG. 7D shows the results of an In vitro cleavage assays measuring proteolytic activity of an anti-IgG affinity series of Fab-MMP3-D4K-4 fusion proteins against a fluorogenic DQ collagen, type IV substrate. Cleavage of the substrates generated fluorescent signal through dequenching of fluorophores.

FIG. 7E shows the results of an In vitro cleavage assays measuring proteolytic activity of an anti-IgG affinity series of Fab-MMP3-D4K-4 fusion proteins against a human IgG1 substrate. Cleavage of IgG was measured with ELISA.

FIG. 7F shows the results of an In vitro cleavage assays measuring proteolytic activity of an anti-IgG affinity series of IgG-MMP3-D4K-4 fusion proteins against a human IgG1 substrate. Cleavage of IgG was measured with ELISA.

FIG. 8A shows the results of cleavage assays measuring the proteolytic activity in human serum of targeted (triangles) and non-targeted (circles) MMP3-D4K-4 against human IgG1 after 24 hours at 37° C. The targeted MMP3-D4K-4 construct contains the anti-IgG Fab RF61-DEF, while the non-targeted construct contains an anti-gD Fab.

FIG. 8B shows the results of cleavage assays measuring the proteolytic activity in human serum of targeted (triangles) and non-targeted (circles) MMP3-D4K-4 against a fluorogenic MMP3 peptide substrate after 24 hours at 37° C. The targeted MMP3-D4K-4 construct contains the anti-IgG Fab RF61-DEF, while the non-targeted construct contains an anti-gD Fab.

FIG. 9 shows the dependence of targeted and non-targeted IgG cleavage on human IgG subtypes. MMP3 does not cleave IgG2, while RF61 does not bind IgG4.

FIG. 10 shows activities of various antibody-MMP3-D₄K-4 fusion formats. After affinity column purification, eluate from each sample was incubated with enterokinase at room temperature overnight, then MMP3-D₄K-4 activity was tested using a fluorogenic peptide substrate (top) and IgG (bottom). Blue (left) and red (right) bars represent samples without and with incubation with enterokinase to cleave the pro-domain, respectively. Background color shading differentiates between IgG and Fab formats, as well as non-targeted formats. Formats with and without antibody domain fusions are separated by a dashed black line. Activity against the peptide substrate (top) demonstrates presence of active MMP3-D₄K-4. All samples containing MMP3 showed some level of enzyme activity. Activity against IgG as measured by ELISA denotes antibody-targeted proteolytic activity. Little to no activity was observed for non-targeted formats, while targeted MMP3-D₄K-4 formats cleaved IgG. The varying extents of activity can be explained by different sample concentrations and purity, as the samples for this initial screening attempt were not normalized or further purified with size exclusion chromatography.

### DETAILED DESCRIPTION

The present application provides fusion proteins comprising an antibody that binds to a target, wherein the antibody is fused to a protease that cleaves a substrate, wherein the target is in the proximity of the substrate. In some embodiments, the protease is fused to one or more polypeptide chains of the antibody. In some embodiments, the target and the substrate are the same molecule.

### I. Definitions

“Affinity” refers to the strength of the sum total of noncovalent interactions between a single binding site of a molecule (e.g., an antibody) and its binding partner (e.g., an antigen). Unless indicated otherwise, as used herein, “binding affinity” refers to intrinsic binding affinity which reflects a 1:1 interaction between members of a binding pair (e.g., antibody and antigen). The affinity of a molecule X for its partner Y can generally be represented by the dissociation constant (K_{D}). Affinity can be measured by common methods known in the art, including those described herein. Specific illustrative and exemplary methods for measuring binding affinity are described in the following.

An “affinity matured” antibody refers to an antibody with one or more alterations in one or more complementary determining regions (CDRs), compared to a parent antibody which does not possess such alterations, such alterations resulting in an improvement in the affinity of the antibody for antigen.

The terms “an antibody that binds to a target” refer to an antibody that is capable of binding the target with sufficient affinity such that the antibody is useful as a diagnostic and/or therapeutic agent in targeting the target. In one aspect, the extent of binding of an antibody to an unrelated, non-target protein is less than about 10% of the binding of the antibody to target as measured, e.g., by surface plasmon resonance (SPR). In certain aspects, an antibody that binds to target has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M). An antibody is said to “specifically bind” to target when the antibody has a K_{D} of 1 μM or less. In certain aspects, an antibody binds to an epitope of the target that is conserved among target from different species.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

The term “epitope” denotes the site on an antigen, either proteinaceous or non-proteinaceous, to which an antibody binds. Epitopes can be formed both from contiguous amino acid stretches (linear epitope) or comprise non-contiguous amino acids (conformational epitope), e.g., coming in spatial proximity due to the folding of the antigen, i.e. by the tertiary folding of a proteinaceous antigen. Linear epitopes are typically still bound by an antibody after exposure of the proteinaceous antigen to denaturing agents, whereas conformational epitopes are typically destroyed upon treatment with denaturing agents. An epitope comprises at least 3, at least 4, at least 5, at least 6, at least 7, or 8-10 amino acids in a unique spatial conformation.

Screening for antibodies binding to a particular epitope (i.e., those binding to the same epitope) can be done using methods routine in the art such as, e.g., without limitation, alanine scanning, peptide blots (see Meth. Mol. Biol. 248 (2004) 443-463), peptide cleavage analysis, epitope excision, epitope extraction, chemical modification of antigens (see Prot. Sci. 9 (2000) 487-496), and cross-blocking (see “Antibodies”, Harlow and Lane (Cold Spring Harbor Press, Cold Spring Harb., NY).

Competitive binding can be used to determine whether an antibody competes for binding with, a reference antibody that binds to the same target. For example, an “antibody that competes for binding with a reference antibody” refers to an antibody that blocks binding of the reference antibody to its antigen in a competition assay by 50% or more, and conversely, the reference antibody blocks binding of the antibody to its antigen in a competition assay by 50% or more. Also for example, to determine if an antibody competes for binding with a reference antibody, the reference antibody is allowed to bind to the target under saturating conditions. After removal of the excess of the reference antibody, the ability of an antibody in question to bind to the target is assessed. If the antibody is able to bind to the target after saturation binding of the reference antibody, it can be concluded that the antibody in question binds to a different epitope than the reference antibody. But, if the antibody in question is not able to bind to the target after saturation binding of the reference antibody, then the antibody in question may bind to the same epitope as the epitope bound by the reference antibody. To confirm whether the antibody in question binds to the same epitope or is just hampered from binding by steric reasons routine experimentation can be used (e.g., peptide mutation and binding analyses using ELISA, RIA, surface plasmon resonance, flow cytometry or any other quantitative or qualitative antibody-binding assay available in the art). This assay should be carried out in two set-ups, i.e. with both of the antibodies being the saturating antibody. If, in both set-ups, only the first (saturating) antibody is capable of binding to the target, then it can be concluded that the antibody in question and the reference antibody compete for binding to the target.

In some aspects, two antibodies are deemed to bind to the same or an overlapping epitope if a 1-, 5-, 10-, 20- or 100-fold excess of one antibody inhibits binding of the other by at least 50%, at least 75%, at least 90% or even 99% or more as measured in a competitive binding assay (see, e.g., Junghans et al., Cancer Res. 50 (1990) 1495-1502).

In some aspects, two antibodies are deemed to bind to the same epitope if essentially all amino acid mutations in the antigen that reduce or eliminate binding of one antibody also reduce or eliminate binding of the other. Two antibodies are deemed to have “overlapping epitopes” if only a subset of the amino acid mutations that reduce or eliminate binding of one antibody reduce or eliminate binding of the other.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. In certain aspects, the antibody is of the IgG₁ isotype. In certain aspects, the antibody is of the IgG₁ isotype with the P329G, L234A and L235A mutation to reduce Fc-region effector function. In other aspects, the antibody is of the IgG₂ isotype. In certain aspects, the antibody is of the IgG₄ isotype with the S228P mutation in the hinge region to improve stability of IgG₄ antibody. The heavy chain constant domains that correspond to the different classes of immunoglobulins are called α, δ, ε, γ, and μ, respectively. The light chain of an antibody may be assigned to one of two types, called kappa (κ) and lambda (λ), based on the amino acid sequence of its constant domain.

The terms “constant region derived from human origin” or “human constant region” as used in the current application denotes a constant heavy chain region of a human antibody of the subclass IgG1, IgG2, IgG3, or IgG4 and/or a constant light chain kappa or lambda region. Such constant regions are well known in the state of the art and e.g. described by Kabat, E. A., et al., Sequences of Proteins of Immunological Interest, 5th ed., Public Health Service, National Institutes of Health, Bethesda, MD (1991) (see also e.g. Johnson, G., and Wu, T. T., Nucleic Acids Res. 28 (2000) 214-218; Kabat, E. A., et al., Proc. Natl. Acad. Sci. USA 72 (1975) 2785-2788). Unless otherwise specified herein, numbering of amino acid residues in the constant region is according to the EU numbering system, also called the EU index of Kabat, as described in Kabat, E. A. et al., Sequences of Proteins of Immunological Interest, 5th ed., Public Health Service, National Institutes of Health, Bethesda, MD (1991), NIH Publication 91-3242.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

An “effective amount” of an agent, e.g., a pharmaceutical composition, refers to an amount effective, at dosages and for periods of time necessary, to achieve the desired therapeutic or prophylactic result.

The term “Fc region” herein is used to define a C-terminal region of an immunoglobulin heavy chain that contains at least a portion of the constant region. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, EU numbering system). Therefore, the C-terminal lysine (Lys447), or the C-terminal glycine (Gly446) and lysine (Lys447), of the Fc region may or may not be present. Amino acid sequences of heavy chains including an Fc region are denoted herein without C-terminal glycine-lysine dipeptide if not indicated otherwise. In one aspect, a heavy chain including an Fc region as specified herein, comprised in an antibody according to the invention, comprises an additional C-terminal glycine-lysine dipeptide (G446 and K447, EU numbering system). In one aspect, a heavy chain including an Fc region as specified herein, comprised in an antibody according to the invention, comprises an additional C-terminal glycine residue (G446, numbering according to EU index). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

“Framework” or “FR” refers to variable domain residues other than complementary determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1(CDR-L1)-FR2-CDR-H2(CDR-L2)-FR3-CDR-H3(CDR-L3)-FR4.

The terms “full length antibody”, “intact antibody”, and “whole antibody” are used herein interchangeably to refer to an antibody having a structure substantially similar to a native antibody structure or having heavy chains that contain an Fc region as defined herein.

The terms “host cell”, “host cell line”, and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Kabat et al., Sequences of Proteins of Immunological Interest, Fifth Edition, NIH Publication 91-3242, Bethesda MD (1991), vols. 1-3. In one aspect, for the VL, the subgroup is subgroup kappa I as in Kabat et al., supra. In one aspect, for the VH, the subgroup is subgroup III as in Kabat et al., supra.

A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

The term “hypervariable region” or “HVR” as used herein refers to each of the regions of an antibody variable domain which are hypervariable in sequence and which determine antigen binding specificity, for example “complementarity determining regions” (“CDRs”).

An “immunoconjugate” is an antibody conjugated to one or more heterologous molecule(s), including but not limited to a cytotoxic agent.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the individual or subject is a human.

An “isolated” antibody is one which has been separated from a component of its natural environment. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007).

The term “nucleic acid molecule” or “polynucleotide” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the invention in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler ert al, Nature Medicine 2017, published online 12 Jun. 2017, doi:10.1038/nm.4356 or EP 2 101 823 B1).

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extrachromosomally or at a chromosomal location that is different from its natural chromosomal location.

“Isolated nucleic acid encoding a fusion protein or a fragment thereof” refers to one or more nucleic acid molecules encoding one or more polypeptides of the fusion proteins or fragment thereof, including such nucleic acid molecule(s) in a single vector or separate vectors, and such nucleic acid molecule(s) present at one or more locations in a host cell.

The term “monoclonal antibody” as used herein refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the present invention may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

“Native antibodies” refer to naturally occurring immunoglobulin molecules with varying structures. For example, native IgG antibodies are heterotetrameric glycoproteins of about 150,000 daltons, composed of two identical light chains and two identical heavy chains that are disulfide-bonded. From N- to C-terminus, each heavy chain has a variable domain (VH), also called a variable heavy domain or a heavy chain variable region, followed by three constant heavy domains (CH1, CH2, and CH3). Similarly, from N- to C-terminus, each light chain has a variable domain (VL), also called a variable light domain or a light chain variable region, followed by a constant light (CL) domain.

The term “package insert” is used to refer to instructions customarily included in commercial packages of therapeutic products, that contain information about the indications, usage, dosage, administration, combination therapy, contraindications and/or warnings concerning the use of such therapeutic products.

The term “pharmaceutical composition” or “pharmaceutical formulation” refers to a preparation which is in such form as to permit the biological activity of an active ingredient contained therein to be effective, and which contains no additional components which are unacceptably toxic to a subject to which the pharmaceutical composition would be administered.

A “pharmaceutically acceptable carrier” refers to an ingredient in a pharmaceutical composition or formulation, other than an active ingredient, which is nontoxic to a subject. A pharmaceutically acceptable carrier includes, but is not limited to, a buffer, excipient, stabilizer, or preservative.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, antibodies of the invention are used to delay development of a disease or to slow the progression of a disease.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementary determining regions (CDRs). (See, e.g., Kindt et al. Kuby Immunology, 6^{th} ed., W.H. Freeman and Co., page 91 (2007).) A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., J. Immunol. 150:880-887 (1993); Clarkson et al., Nature 352:624-628 (1991).

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

### II. Compositions and Methods

In one aspect, the invention is based, in part, on the remarkable findings that fusion proteins that have an antibody that binds to a target fused with a protease that cleaves a substrate are able to catalyze the cleavage in a much more potent manner. In some embodiments, the target and the substrate are in proximity. In some embodiments, the target and the substrate are the same molecule. In some aspects, the fusions proteins are antibody-enzyme fusions that provide favorable selectivity of an antibody to the enzyme to improve catalytic activity and tune selectivity. In another aspect of the invention, the invention is based on engineering fusions proteins (e.g. antibody-enzyme fusions) to broaden the therapeutic range of proteases. In another aspect of the invention, the antibody-enzyme fusions provide for a new therapeutic approach for neutralization of therapeutic targets (e.g., those of high abundance). In another aspect of the invention, the antibody-enzyme fusions provide for improvements in delivery of proteases, e.g., in hard-to-reach subcellular tissue sites. In another aspect of the invention, the antibody-enzyme fusions provide for concentrating the proteases at one or more sites. In another aspect of the invention, the antibody-enzyme fusions decrease non-specific activity (e.g. protease activity at off-target sites) of proteases. In another aspect of the invention, the invention is based on targeting proteins with high protein content to effectively lower the amount therapeutically. Fusion proteins of the invention are useful, e.g., for the treatment of various diseases or conditions that involve a therapeutic substrate desired to be removed. The fusion proteins are also effective in several systems and tissue types, such as hard-to-reach locations including the central nervous system.

### A. Fusion Proteins

The fusion proteins described herein bring together a protease with a substrate that is to be targeted for proteolysis. To facilitate proteolysis of a target, the fusion protein comprises an antibody that binds to the target, and an enzyme that catalyzes hydrolysis of a substrate. These groups can be fused directly or via a linker. This molecular construct can bring the enzyme in specific proximity with the target so that it is specifically proteolyzed by the enzyme.

Provided herein are fusion proteins comprising an antibody that binds to a target, wherein the antibody is fused to a protease that cleaves a substrate, wherein the target is in the proximity of the substrate. In some embodiments, the target and the substrate are the same molecule. Fusion proteins described herein have one or more of the following advantages: a) higher specificity against the desired substrate, b) increased potency against the substrate, c) longer half-life, d) more efficient removal of the substrates. Those advantages allow the fusion proteins to more effectively suppress therapeutic targets.

In some embodiments, there is provided a fusion protein comprising an antibody that binds to a target, wherein the antibody is fused to a protease that cleaves a substrate, wherein the target and the substrate are the same molecule, wherein the antibody comprises a) a first polypeptide comprising a heavy chain variable (VH) domain and a first heavy chain constant (CH1) domain and b) a second polypeptide comprising a light chain variable (VL) domain and a light chain constant (CL) domain, wherein the protease is fused to the N-terminal of the VL domain, wherein the VH domain and VL domain form a binding domain for the target, wherein a disulfide bond is formed between the CH1 domain and the CL domain. In some embodiments, the antibody does not comprise a Fc fragment. In some embodiments, the protease is fused with the antibody via a linker. In some embodiments, the linker is a peptide linker, optionally wherein the linker has a length of at least about 5, 10, 15, 20, 25, or 30 amino acids. In some embodiments, the linker is cleavable by a protease that is distinct from the protease comprised in the fusion protein. In some embodiments, the linker is not cleavable. In some embodiments, the dissociation rate constant (k_{off}) of antibody to the target is no more than about 5×10⁻³, 1×10⁻³, 5×10⁻⁴, or 1×10⁻⁴. In some embodiments, the equilibrium dissociation constant (k_{D}) of the antibody to the target has a range of about 0.1 nM to about 1000 nM. In some embodiments, the target and the substrate are expressed in the central nervous system (CNS). In some embodiments, the target and the substrate are the same molecule. In some embodiments, the protease is a metalloprotease.

In some embodiments, there is provided a fusion protein comprising an antibody that binds to a target, wherein the antibody is fused to a protease that cleaves a substrate, wherein the target and the substrate are the same molecule, wherein the antibody comprises a) a first polypeptide comprising a light chain variable (VL) domain and a light chain constant (CL) domain, b) a second polypeptide comprising a heavy chain variable (VH) domain and first heavy chain constant (CH1) domains, and c) a third polypeptide comprising a Fc region, wherein the protease is fused to the N-terminus of the third polypeptide, and wherein the VH domain and the VL domain form a binding domain for the target. In some embodiments, the protease is fused with the antibody via a linker. In some embodiments, the linker is a peptide linker, optionally wherein the linker has a length of at least about 5, 10, 15, 20, 25, or 30 amino acids. In some embodiments, the linker is cleavable by a protease that is distinct from the protease comprised in the fusion protein. In some embodiments, the linker is not cleavable. In some embodiments, the dissociation rate constant (k_{off}) of antibody to the target is no more than about 5×10⁻³, 1×10⁻³, 5×10⁻⁴, or 1×10⁻⁴. In some embodiments, the equilibrium dissociation constant (k_{D}) of the antibody to the target has a range of about 0.1 nM to about 1000 nM. In some embodiments, the target and the substrate are expressed in the central nervous system (CNS). In some embodiments, the target and the substrate are the same molecule. In some embodiments, the protease is a metalloprotease.

In some embodiments, there is provided a fusion protein comprising an antibody that binds to a target, wherein the antibody is fused to a protease that cleaves a substrate, wherein the target and the substrate are the same molecule, wherein the antibody comprises two heavy chains and two light chains, wherein each heavy chain comprises a heavy chain variable (VH) domain and a heavy chain constant (CH) domain, wherein each light chain comprises a light chain variable (VL) domain and a light chain constant (CL) domain, wherein the protease is fused to the N-terminus of the two light chains. In some embodiments, the protease is fused with the antibody via a linker. In some embodiments, the linker is a peptide linker, optionally wherein the linker has a length of at least about 5, 10, 15, 20, 25, or 30 amino acids. In some embodiments, the linker is cleavable by a protease that is distinct from the protease comprised in the fusion protein. In some embodiments, the linker is not cleavable. In some embodiments, the dissociation rate constant (k_{off}) of antibody to the target is no more than about 5×10⁻³, 1×10⁻³, 5×10⁻⁴, or 1×10⁻⁴. In some embodiments, the equilibrium dissociation constant (k_{D}) of the antibody to the target has a range of about 0.1 nM to about 1000 nM. In some embodiments, the target and the substrate are expressed in the central nervous system (CNS). In some embodiments, the target and the substrate are the same molecule. In some embodiments, the protease is a metalloprotease.

In some embodiments, there is provided a fusion protein comprising an antibody that binds to a target, wherein the antibody is fused to a protease that cleaves a substrate, wherein the target and the substrate are the same molecule, wherein the antibody comprises two heavy chains and two light chains, wherein each heavy chain comprises a heavy chain variable (VH) domain and a heavy chain constant (CH) domain, wherein each light chain comprises a light chain variable (VL) domain and a light chain constant (CL) domain, wherein the protease is fused to the N-terminus of the two heavy chains. In some embodiments, the protease is fused with the antibody via a linker. In some embodiments, the linker is a peptide linker, optionally wherein the linker has a length of at least about 5, 10, 15, 20, 25, or 30 amino acids. In some embodiments, the linker is cleavable by a protease that is distinct from the protease comprised in the fusion protein. In some embodiments, the linker is not cleavable. In some embodiments, the dissociation rate constant (k_{off}) of antibody to the target is no more than about 5×10⁻³, 1×10⁻³, 5×10⁻⁴, or 1×10⁻⁴. In some embodiments, the equilibrium dissociation constant (k_{D}) of the antibody to the target has a range of about 0.1 nM to about 1000 nM. In some embodiments, the target and the substrate are expressed in the central nervous system (CNS). In some embodiments, the target and the substrate are the same molecule. In some embodiments, the protease is a metalloprotease.

In some embodiments, there is provided a fusion protein comprising an antibody that binds to a target, wherein the antibody is fused to a protease that cleaves a substrate, wherein the target and the substrate are the same molecule, wherein the antibody comprises two heavy chains and two light chains, wherein each heavy chain comprises a heavy chain variable (VH) domain and a heavy chain constant (CH) domain, wherein each light chain comprises a light chain variable (VL) domain and a light chain constant (CL) domain, wherein the protease is fused to the N-terminus of the two light chains and the N-terminus of the two heavy chains. In some embodiments, the protease is fused with the antibody via a linker. In some embodiments, the linker is a peptide linker, optionally wherein the linker has a length of at least about 5, 10, 15, 20, 25, or 30 amino acids. In some embodiments, the linker is cleavable by a protease that is distinct from the protease comprised in the fusion protein. In some embodiments, the linker is not cleavable. In some embodiments, the dissociation rate constant (k_{off}) of antibody to the target is no more than about 5×10⁻³, 1×10⁻³, 5×10⁻⁴, or 1×10⁻⁴. In some embodiments, the equilibrium dissociation constant (k_{D}) of the antibody to the target has a range of about 0.1 nM to about 1000 nM. In some embodiments, the target and the substrate are expressed in the central nervous system (CNS). In some embodiments, the target and the substrate are the same molecule. In some embodiments, the protease is a metalloprotease.

In some embodiments, there is provided a fusion protein comprising an antibody that binds to a target, wherein the antibody is fused to a protease that cleaves a substrate, wherein the target and the substrate are the same molecule, wherein the antibody comprises two heavy chains and two light chains, wherein each heavy chain comprises a heavy chain variable (VH) domain and a heavy chain constant (CH) domain, wherein each light chain comprises a light chain variable (VL) domain and a light chain constant (CL) domain, wherein the protease is fused to the C-terminus of one of the heavy chains. In some embodiments, the protease is fused with the antibody via a linker. In some embodiments, the linker is a peptide linker, optionally wherein the linker has a length of at least about 5, 10, 15, 20, 25, or 30 amino acids. In some embodiments, the linker is cleavable by a protease that is distinct from the protease comprised in the fusion protein. In some embodiments, the linker is not cleavable. In some embodiments, the dissociation rate constant (k_{off}) of antibody to the target is no more than about 5×10⁻³, 1×10⁻³, 5×10⁻⁴, or 1×10⁻⁴. In some embodiments, the equilibrium dissociation constant (k_{D}) of the antibody to the target has a range of about 0.1 nM to about 1000 nM. In some embodiments, the target and the substrate are expressed in the central nervous system (CNS). In some embodiments, the target and the substrate are the same molecule. In some embodiments, the protease is a metalloprotease.

In some embodiments, there is provided a fusion protein comprising an antibody that binds to a target, wherein the antibody is fused to a protease that cleaves a substrate, wherein the target and the substrate are the same molecule, wherein the antibody comprises two heavy chains and two light chains, wherein each heavy chain comprises a heavy chain variable (VH) domain and a heavy chain constant (CH) domain, wherein each light chain comprises a light chain variable (VL) domain and a light chain constant (CL) domain, wherein the protease is fused to the C-terminus of both of the heavy chains. In some embodiments, the protease is fused with the antibody via a linker. In some embodiments, the linker is a peptide linker, optionally wherein the linker has a length of at least about 5, 10, 15, 20, 25, or 30 amino acids. In some embodiments, the linker is cleavable by a protease that is distinct from the protease comprised in the fusion protein. In some embodiments, the linker is not cleavable. In some embodiments, the dissociation rate constant (k_{off}) of antibody to the target is no more than about 5×10⁻³, 1×10⁻³, 5×10⁻⁴, or 1×10⁻⁴. In some embodiments, the equilibrium dissociation constant (k_{D}) of the antibody to the target has a range of about 10 nM to about 1000 nM. In some embodiments, the target and the substrate are expressed in the central nervous system (CNS). In some embodiments, the target and the substrate are the same molecule. In some embodiments, the protease is a metalloprotease.

In some embodiments, there is provided a fusion protein comprising an antibody that binds to a target, wherein the antibody is fused to a protease that cleaves a substrate, wherein the target and the substrate are in the proximity of each other or are the same molecule expressed in the central nervous system (CNS). In some embodiments, the antibody comprises a full-length antibody. In some embodiments, the protease is fused to the N-terminus and/or C-terminus of one or more polypeptide chains of the antibody. In some embodiments, the antibody comprises an antibody fragment. In some embodiments, the protease is fused with the antibody via a linker. In some embodiments, the linker is a peptide linker, optionally wherein the linker has a length of at least about 5, 10, 15, 20, 25, or 30 amino acids. In some embodiments, the linker is cleavable by a protease that is distinct from the protease comprised in the fusion protein. In some embodiments, the linker is not cleavable. In some embodiments, the dissociation rate constant (k_{off}) of antibody to the target is no more than about 5×10⁻³, 1×10⁻³, 5×10⁻⁴, or 1×10⁻⁴. In some embodiments, the equilibrium dissociation constant (k_{D}) of the antibody to the target has a range of about 0.1 nM to about 1000 nM. In some embodiments, the target and the substrate are expressed in the central nervous system (CNS). In some embodiments, the target and the substrate are the same molecule. In some embodiments, the protease is a metalloprotease.

In some embodiments, there is provided a fusion protein comprising an antibody that binds to a target, wherein the antibody is fused to a protease that cleaves a substrate, wherein the target and the substrate are in the proximity of each other or are the same molecule expressed in the circulation. In some embodiments, the antibody comprises a full-length antibody. In some embodiments, the protease is fused to the N-terminus and/or C-terminus of one or more polypeptide chains of the antibody. In some embodiments, the antibody comprises an antibody fragment. In some embodiments, the protease is fused with the antibody via a linker. In some embodiments, the linker is a peptide linker, optionally wherein the linker has a length of at least about 5, 10, 15, 20, 25, or 30 amino acids. In some embodiments, the linker is cleavable by a protease that is distinct from the protease comprised in the fusion protein. In some embodiments, the linker is not cleavable. In some embodiments, the dissociation rate constant (k_{off}) of antibody to the target is no more than about 5×10⁻³, 1×10⁻³, 5×10⁻⁴, or 1×10⁻⁴. In some embodiments, the equilibrium dissociation constant (k_{D}) of the antibody to the target has a range of about 0.1 nM to about 1000 nM. In some embodiments, the target and the substrate are expressed in the central nervous system (CNS). In some embodiments, the target and the substrate are the same molecule. In some embodiments, the protease is a metalloprotease.

In some embodiments, there is provided a fusion protein comprising an antibody that binds to a target, wherein the antibody is fused to a protease that cleaves a substrate, wherein the target and the substrate are in the proximity of each other or are the same molecule expressed in a cancer tissue. In some embodiments, the antibody comprises a full-length antibody. In some embodiments, the protease is fused to the N-terminus and/or C-terminus of one or more polypeptide chains of the antibody. In some embodiments, the antibody comprises an antibody fragment. In some embodiments, the protease is fused with the antibody via a linker. In some embodiments, the linker is a peptide linker, optionally wherein the linker has a length of at least about 5, 10, 15, 20, 25, or 30 amino acids. In some embodiments, the linker is cleavable by a protease that is distinct from the protease comprised in the fusion protein. In some embodiments, the linker is not cleavable. In some embodiments, the dissociation rate constant (k_{off}) of antibody to the target is no more than about 5×10⁻³, 1×10⁻³, 5×10⁻⁴, or 1×10⁻⁴. In some embodiments, the equilibrium dissociation constant (k_{D}) of the antibody to the target has a range of about 0.1 nM to about 1000 nM. In some embodiments, the target and the substrate are expressed in the central nervous system (CNS). In some embodiments, the target and the substrate are the same molecule. In some embodiments, the protease is a metalloprotease.

In some embodiments, there is provided a fusion protein comprising an antibody that binds to a target, wherein the antibody is fused to a protease that cleaves a substrate, wherein the target and the substrate are in the proximity of each other or are the same molecule expressed in a diseased tissue (e.g., with inflammation). In some embodiments, the antibody comprises a full-length antibody. In some embodiments, the protease is fused to the N-terminus and/or C-terminus of one or more polypeptide chains of the antibody. In some embodiments, the antibody comprises an antibody fragment. In some embodiments, the protease is fused with the antibody via a linker. In some embodiments, the linker is a peptide linker, optionally wherein the linker has a length of at least about 5, 10, 15, 20, 25, or 30 amino acids. In some embodiments, the linker is cleavable by a protease that is distinct from the protease comprised in the fusion protein. In some embodiments, the linker is not cleavable. In some embodiments, the dissociation rate constant (k_{off}) of antibody to the target is no more than about 5×10⁻³, 1×10⁻³, 5×10⁻⁴, or 1×10⁻⁴. In some embodiments, the equilibrium dissociation constant (k_{D}) of the antibody to the target has a range of about 0.1 nM to about 1000 nM. In some embodiments, the target and the substrate are expressed in the central nervous system (CNS). In some embodiments, the target and the substrate are the same molecule. In some embodiments, the protease is a metalloprotease.

In some embodiments, there is provided a fusion protein comprising an antibody that binds to a target, wherein the antibody is fused to a protease that cleaves a substrate, wherein the target and the substrate are in the proximity of each other or are the same molecule expressed in a fibrosis tissue. In some embodiments, the antibody comprises a full-length antibody. In some embodiments, the protease is fused to the N-terminus and/or C-terminus of one or more polypeptide chains of the antibody. In some embodiments, the antibody comprises an antibody fragment. In some embodiments, the protease is fused with the antibody via a linker. In some embodiments, the linker is a peptide linker, optionally wherein the linker has a length of at least about 5, 10, 15, 20, 25, or 30 amino acids. In some embodiments, the linker is cleavable by a protease that is distinct from the protease comprised in the fusion protein. In some embodiments, the linker is not cleavable. In some embodiments, the dissociation rate constant (k_{off}) of antibody to the target is no more than about 5×10⁻³, 1×10⁻³, 5×10⁻⁴, or 1×10⁻⁴. In some embodiments, the equilibrium dissociation constant (k_{D}) of the antibody to the target has a range of about 0.1 nM to about 1000 nM. In some embodiments, the target and the substrate are expressed in the central nervous system (CNS). In some embodiments, the target and the substrate are the same molecule. In some embodiments, the protease is a metalloprotease.

In some embodiments, there is provided a fusion protein comprising an antibody that binds to a target, wherein the antibody is fused to a protease that cleaves a substrate, wherein the target and the substrate are associated with a neurological disease. In some embodiments, the target and the substrate are amyloid-β (Aβ). In some embodiments, the antibody and/or the protease targets both Aβ₁₋₄₀ and Aβ₁₋₄₂. In some embodiments, the antibody is crenezumab or a variant thereof, or solanezumab or a variant thereof, optionally wherein the variant of crenezumb comprises G33S on its heavy chains, and further optionally wherein the variant of crenezumb comprises S56F on its light chains. In some embodiments, the protease is selected from the group consisting of neprolysin (NEP), neprolysin-2 (NEP2), endothelin-converting enzyme 1 and 2 (ECE1 and ECE2), angiotensin-converting enzyme (ACE), insulin-degrading enzyme (IDE), matrix metalloproteinase 2 and 9 (MMP2 and MMP9), and matriptase (MTSP1). In some embodiments, the protease is neprolysin. In some embodiments, the antibody comprises two heavy chains and two light chains, wherein the protease is fused to the C-terminus of one or both heavy chains of the antibody.

In some embodiments, there is provided a fusion protein comprising an antibody that binds to a target, wherein the antibody is fused to a protease that cleaves a substrate, wherein the target and the substrate are IgG. In some embodiments, the protease is selected from the group consisting of matrix metalloproteinase 3 (MMP3), MMP7, Cathepsin G or a variant thereof. In some embodiments, the protease is MMP3 or a variant thereof. In some embodiments, the antibody comprises a full-length antibody. In some embodiments, the antibody comprises a R335E mutation in the Fc Fragment. In some embodiments, the full-length antibody comprises a hinge region resistant to the protease, optionally wherein the hinge region comprises a (G₄A)₂. In some embodiments, the antibody comprises a Fab fragment. In some embodiments, the antibody comprises a Rheumatoid factor or a variant thereof.

### Antibodies

The antibodies described herein can be monoclonal antibodies (including full-length monoclonal antibodies), polyclonal antibodies, multispecific antibodies (e.g., bispecific antibodies), and antibody fragments so long as they exhibit the desired biological activity (e.g., binding to the target).

In some aspects, the antibody is or comprises a full-length antibody. In some embodiments, the antibody is an intact IgA, IgG, IgM, IgD, IgE antibody or other antibody class or isotype as defined herein.

In some aspects, the antibody is chimeric, human, partially humanized, fully humanized, or semi-synthetic. Antibodies and/or antibody fragments described in may be derived from murine antibodies, rabbit antibodies, human antibodies, fully humanized antibodies, camelid antibody variable domains and humanized versions, shark antibody variable domains and humanized versions, and camelized antibody variable domains.

In some aspects, the antibody comprises an Fc fragment. In some aspects, the Fc fragment is selected from the group consisting of Fc fragments from IgG, IgA, IgD, IgE, IgM, and combinations and hybrids thereof. In some embodiments, the Fc fragment is derived from a human IgG. In some embodiments, the Fc fragment comprises the Fc region of human IgG1, IgG2, IgG3, IgG4, or a combination or hybrid IgG.

In some aspects, the antibody binds to a target that is the same as, or a fragment of the substrate. In some aspects, the antibody binds to an isoform (e.g. protein variant) of the target. Therefore, in some aspects, the antibody recognizes one isoform. In some aspects, the antibody recognizes one or more isoforms. In some aspects, the antibody prefers one isoform. Preference may be exhibited by enhanced binding and other parameters that may be measured by biochemical assays, such as the assays described herein. In some aspects, the antibody prefers one or more isoform. In some aspects, the antibody is not isoform specific. In some aspects, the antibody binds to all isoforms. In some aspects, the antibody does not exhibit superior binding to any isoform of the target.

In some embodiments, the target is an extracellular molecule. In some embodiments, the target is a molecule in the tissue. In some embodiments, the target is a molecule in the tissue in the central nervous system (e.g., in brain), and optionally the antibody penetrates the blood-brain membrane. See e.g., Fluids Barriers CNS. 2022 Dec. 12; 19(1):99. In some embodiments, the target is in circulation. In some embodiments, the target is in an organ (e.g., liver, spleen, lung, heart, etc).

In some embodiments, the target is an intracellular molecule.

In some embodiments, the target is a cell surface molecule.

In some embodiments, the target is a peptide or a fragment thereof. In some embodiments, the peptide has a length of about 0-100 amino acids, 10-80 amino acids, 20-70 amino acids, or 30-60 amino acids.

In a further aspect, the target is amyloid beta (ap or abeta).

In a further aspect, the target is immunoglobulin G (IgG).

In a further aspect, an antibody that binds to a target according to any of the above aspects may incorporate any of the features, singly or in combination, as described in Sections 1-7 below:

### 1. Antibody Affinity

In certain aspects, an antibody provided herein has a dissociation constant (KD) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M).

In certain aspects, an antibody provided herein has a dissociation constant (KD) between 100 nM and 1 μM, between 10 nM and 100 nM, between 1 nM and 10 nM, between 0.1 nM and 1 nM, between 0.01 nM and 0.1 nM, or between 0.001 and 0.1 nM.

In certain aspects, an antibody provided herein has one or more modifications that promotes a weaker dissociation constant against the target.

In certain aspects, an antibody provided herein has a off-rate constant (k_{off}) of ≤10⁻² s⁻¹, ≤5×10⁻³ s⁻¹, ≤10⁻³ s⁻¹, ≤5×10⁻⁴ s⁻¹, ≤10⁻⁴ s⁻¹, ≤5×10⁻⁵ s⁻¹, or ≤10⁻⁵ s⁻¹. In certain aspects, an antibody provided herein has a off-rate (k_{off}) between 10⁻² s⁻¹ and 5×10⁻³ s⁻¹, between 10⁻³ s⁻¹ and 5×10⁻³ s⁻¹, between 5×10⁻⁴ s⁻¹ and 10⁻³ s⁻¹, between 10⁻⁴ s⁻¹ and 5×10⁻⁴ s⁻¹, between 10⁻⁴ s⁻¹ and 5×10⁻⁴ s⁻¹, between 5×10⁻⁵ s⁻¹ and 10⁻⁴ s⁻¹ or between 10⁻⁵ s⁻¹ and 5×10⁻⁵ s⁻¹.

In certain aspects, an antibody provided herein has one or more modifications that promotes a quicker off-rate constant against the target.

In one aspect, K_{D} is measured using a BIACORE® surface plasmon resonance assay. For example, an assay using a BIACORE®-2000 or a BIACORE®-3000 (BIAcore, Inc., Piscataway, NJ) is performed at 25° C. with immobilized antigen CM5 chips at ˜10 response units (RU). In one aspect, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{D}) is calculated as the ratio k_{off}/kₒₙ. See, e.g., Chen et al., J. Mol. Biol. 293:865-881 (1999). If the on-rate exceeds 10⁶ M⁻¹ s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

In an alternative method, K_{D} is measured by a radiolabeled antigen binding assay (RIA). In one aspect, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

### 2. Antibody Fragments

In certain aspects, an antibody provided herein is an antibody fragment.

In one aspect, the antibody fragment is a Fab, Fab′, Fab′-SH, or F(ab′)₂ fragment, in particular a Fab fragment. Papain digestion of intact antibodies produces two identical antigen-binding fragments, called “Fab” fragments containing each the heavy- and light-chain variable domains (VH and VL, respectively) and also the constant domain of the light chain (CL) and the first constant domain of the heavy chain (CH1). The term “Fab fragment” thus refers to an antibody fragment comprising a light chain comprising a VL domain and a CL domain, and a heavy chain fragment comprising a VH domain and a CH1 domain. “Fab′ fragments” differ from Fab fragments by the addition of residues at the carboxy terminus of the CH1 domain including one or more cysteines from the antibody hinge region. Fab′-SH are Fab′ fragments in which the cysteine residue(s) of the constant domains bear a free thiol group. Pepsin treatment yields an F(ab′)₂ fragment that has two antigen-binding sites (two Fab fragments) and a part of the Fc region. For discussion of Fab and F(ab′)₂ fragments comprising salvage receptor binding epitope residues and having increased in vivo half-life, see U.S. Pat. No. 5,869,046.

In another aspect, the antibody fragment is a diabody, a triabody or a tetrabody. “Diabodies” are antibody fragments with two antigen-binding sites that may be bivalent or bispecific. See, for example, EP 404,097; WO 1993/01161; Hudson et al., Nat. Med. 9:129-134 (2003); and Hollinger et al., Proc. Natl. Acad. Sci. USA 90: 6444-6448 (1993). Triabodies and tetrabodies are also described in Hudson et al., Nat. Med. 9:129-134 (2003).

In a further aspect, the antibody fragment is a single chain Fab fragment. A “single chain Fab fragment” or “scFab” is a polypeptide consisting of an antibody heavy chain variable domain (VH), an antibody heavy chain constant domain 1 (CH1), an antibody light chain variable domain (VL), an antibody light chain constant domain (CL) and a linker, wherein said antibody domains and said linker have one of the following orders in N-terminal to C-terminal direction: a) VH-CH1-linker-VL-CL, b) VL-CL-linker-VH-CH1, c) VH-CL-linker-VL-CH1 or d) VL-CH1-linker-VH-CL. In particular, said linker is a polypeptide of at least 30 amino acids, preferably between 32 and 50 amino acids. Said single chain Fab fragments are stabilized via the natural disulfide bond between the CL domain and the CH1 domain. In addition, these single chain Fab fragments might be further stabilized by generation of interchain disulfide bonds via insertion of cysteine residues (e.g., position 44 in the variable heavy chain and position 100 in the variable light chain according to Kabat numbering).

In another aspect, the antibody fragment is single-chain variable fragment (scFv). A “single-chain variable fragment” or “scFv” is a fusion protein of the variable domains of the heavy (VH) and light chains (VL) of an antibody, connected by a linker. In particular, the linker is a short polypeptide of 10 to 25 amino acids and is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458.

In another aspect, the antibody fragment is a single-domain antibody. “Single-domain antibodies” are antibody fragments comprising all or a portion of the heavy chain variable domain or all or a portion of the light chain variable domain of an antibody. In certain aspects, a single-domain antibody is a human single-domain antibody (Domantis, Inc., Waltham, MA; see, e.g., U.S. Pat. No. 6,248,516 B1).

Antibody fragments can be made by various techniques, including but not limited to proteolytic digestion of an intact antibody as well as recombinant production by recombinant host cells (e.g., E. coli), as described herein.

### 3. Chimeric and Humanized Antibodies

In certain aspects, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain aspects, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which the CDRs (or portions thereof) are derived from a non-human antibody, and FRs (or portions thereof) are derived from human antibody sequences. A humanized antibody optionally will also comprise at least a portion of a human constant region. In some aspects, some FR residues in a humanized antibody are substituted with corresponding residues from a non-human antibody (e.g., the antibody from which the CDR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### 4. Human Antibodies

In certain aspects, an antibody provided herein is a human antibody. Human antibodies can be produced using various techniques known in the art. Human antibodies are described generally in van Dijk and van de Winkel, Curr. Opin. Pharmacol. 5: 368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extrachromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; U.S. Pat. No. 5,770,429 describing HUMAB® technology; U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods. Human myeloma and mouse-human heteromyeloma cell lines for the production of human monoclonal antibodies have been described. (See, e.g., Kozbor J. Immunol., 133: 3001 (1984); Brodeur et al., Monoclonal Antibody Production Techniques and Applications, pp. 51-63 (Marcel Dekker, Inc., New York, 1987); and Boerner et al., J. Immunol., 147: 86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26(4):265-268 (2006) (describing human-human hybridomas). Human hybridoma technology (Trioma technology) is also described in Vollmers and Brandlein, Histology and Histopathology, 20(3):927-937 (2005) and Vollmers and Brandlein, Methods and Findings in Experimental and Clinical Pharmacology, 27(3):185-91 (2005).

Human antibodies may also be generated by isolating variable domain sequences selected from human-derived phage display libraries. Such variable domain sequences may then be combined with a desired human constant domain. Techniques for selecting human antibodies from antibody libraries are described below.

### 5. Library-Derived Antibodies

In certain aspects, an antibody provided herein is derived from a library. Antibodies of the invention may be isolated by screening combinatorial libraries for antibodies with the desired activity or activities. Methods for screening combinatorial libraries are reviewed, e.g., in Lerner et al. in Nature Reviews 16:498-508 (2016). For example, a variety of methods are known in the art for generating phage display libraries and screening such libraries for antibodies possessing the desired binding characteristics. Such methods are reviewed, e.g., in Frenzel et al. in mAbs 8:1177-1194 (2016); Bazan et al. in Human Vaccines and Immunotherapeutics 8:1817-1828 (2012) and Zhao et al. in Critical Reviews in Biotechnology 36:276-289 (2016) as well as in Hoogenboom et al. in Methods in Molecular Biology 178:1-37 (O'Brien et al., ed., Human Press, Totowa, NJ, 2001) and in Marks and Bradbury in Methods in Molecular Biology 248:161-175 (Lo, ed., Human Press, Totowa, NJ, 2003).

In certain phage display methods, repertoires of VH and VL genes are separately cloned by polymerase chain reaction (PCR) and recombined randomly in phage libraries, which can then be screened for antigen-binding phage as described in Winter et al. in Annual Review of Immunology 12: 433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al. in EMBO Journal 12: 725-734 (1993). Furthermore, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter in Journal of Molecular Biology 227: 381-388 (1992). Patent publications describing human antibody phage libraries include, for example: U.S. Pat. Nos. 5,750,373; 7,985,840; 7,785,903 and 8,679,490 as well as US Patent Publication Nos. 2005/0079574, 2007/0117126, 2007/0237764 and 2007/0292936.

Further examples of methods known in the art for screening combinatorial libraries for antibodies with a desired activity or activities include ribosome and mRNA display, as well as methods for antibody display and selection on bacteria, mammalian cells, insect cells or yeast cells. Methods for yeast surface display are reviewed, e.g., in Scholler et al. in Methods in Molecular Biology 503:135-56 (2012) and in Cherf et al. in Methods in Molecular biology 1319:155-175 (2015) as well as in Zhao et al. in Methods in Molecular Biology 889:73-84 (2012). Methods for ribosome display are described, e.g., in He et al. in Nucleic Acids Research 25:5132-5134 (1997) and in Hanes et al. in PNAS 94:4937-4942 (1997).

Antibodies or antibody fragments isolated from human antibody libraries are considered human antibodies or human antibody fragments herein.

### 6. Multispecific Antibodies

In certain aspects, an antibody provided herein is a multispecific antibody, e.g., a bispecific antibody. “Multispecific antibodies” are monoclonal antibodies that have binding specificities for at least two different sites, i.e., different epitopes on different antigens or different epitopes on the same antigen. In certain aspects, the multispecific antibody has three or more binding specificities. In certain aspects, one of the binding specificities is for a target and the other specificity is for any other antigen. In certain aspects, bispecific antibodies may bind to two (or more) different epitopes of a target. Multispecific (e.g., bispecific) antibodies may also be used to localize cytotoxic agents or cells to cells which express a target. Multispecific antibodies may be prepared as full length antibodies or antibody fragments.

Techniques for making multispecific antibodies include, but are not limited to, recombinant co-expression of two immunoglobulin heavy chain-light chain pairs having different specificities (see Milstein and Cuello, Nature 305: 537 (1983)) and “knob-in-hole” engineering (see, e.g., U.S. Pat. No. 5,731,168, and Atwell et al., J. Mol. Biol. 270:26 (1997)). Multi-specific antibodies may also be made by engineering electrostatic steering effects for making antibody Fc-heterodimeric molecules (see, e.g., WO 2009/089004); cross-linking two or more antibodies or fragments (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992) and WO 2011/034605); using the common light chain technology for circumventing the light chain mis-pairing problem (see, e.g., WO 98/50431); using “diabody” technology for making bispecific antibody fragments (see, e.g., Hollinger et al., Proc. Natl. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147: 60 (1991).

Engineered antibodies with three or more antigen binding sites, including for example, “Octopus antibodies”, or DVD-Ig are also included herein (see, e.g., WO 2001/77342 and WO 2008/024715). Other examples of multispecific antibodies with three or more antigen binding sites can be found in WO 2010/115589, WO 2010/112193, WO 2010/136172, WO 2010/145792, and WO 2013/026831. The bispecific antibody or antigen binding fragment thereof also includes a “Dual Acting FAb” or “DAF” comprising an antigen binding site that binds to a target as well as another different antigen, or two different epitopes of a target (see, e.g., US 2008/0069820 and WO 2015/095539).

Multi-specific antibodies may also be provided in an asymmetric form with a domain crossover in one or more binding arms of the same antigen specificity, i.e. by exchanging the VH/VL domains (see e.g., WO 2009/080252 and WO 2015/150447), the CH1/CL domains (see e.g., WO 2009/080253) or the complete Fab arms (see e.g., WO 2009/080251, WO 2016/016299, also see Schaefer et al, PNAS, 108 (2011) 1187-1191, and Klein at al., MAbs 8 (2016) 1010-20). In one aspect, the multispecific antibody comprises a cross-Fab fragment. The term “cross-Fab fragment” or “xFab fragment” or “crossover Fab fragment” refers to a Fab fragment, wherein either the variable regions or the constant regions of the heavy and light chain are exchanged. A cross-Fab fragment comprises a polypeptide chain composed of the light chain variable region (VL) and the heavy chain constant region 1 (CH1), and a polypeptide chain composed of the heavy chain variable region (VH) and the light chain constant region (CL). Asymmetrical Fab arms can also be engineered by introducing charged or non-charged amino acid mutations into domain interfaces to direct correct Fab pairing. See e.g., WO 2016/172485.

Various further molecular formats for multispecific antibodies are known in the art and are included herein (see e.g., Spiess et al., Mol Immunol 67 (2015) 95-106).

A particular type of multispecific antibodies, also included herein, are bispecific antibodies designed to simultaneously bind to a surface antigen on a target cell, e.g., a tumor cell, and to an activating, invariant component of the T cell receptor (TCR) complex, such as CD3, for retargeting of T cells to kill target cells. Hence, in certain aspects, an antibody provided herein is a multispecific antibody, particularly a bispecific antibody, wherein one of the binding specificities is for a target and the other is for a different target (e.g. another antigen).

Examples of bispecific antibody formats that may be useful for this purpose include, but are not limited to, the so-called “BiTE” (bispecific T cell engager) molecules wherein two scFv molecules are fused by a flexible linker (see, e.g., WO 2004/106381, WO 2005/061547, WO 2007/042261, and WO 2008/119567, Nagorsen and Bauerle, Exp Cell Res 317, 1255-1260 (2011)); diabodies (Holliger et al., Prot Eng 9, 299-305 (1996)) and derivatives thereof, such as tandem diabodies (“TandAb”; Kipriyanov et al., J Mol Biol 293, 41-56 (1999)); “DART” (dual affinity retargeting) molecules which are based on the diabody format but feature a C-terminal disulfide bridge for additional stabilization (Johnson et al., J Mol Biol 399, 436-449 (2010)), and so-called triomabs, which are whole hybrid mouse/rat IgG molecules (reviewed in Seimetz et al., Cancer Treat Rev 36, 458-467 (2010)). Particular T cell bispecific antibody formats included herein are described in WO 2013/026833, WO 2013/026839, WO 2016/020309; Bacac et al., Oncoimmunology 5(8) (2016) e1203498.

### 7. Antibody Variants

In certain aspects, amino acid sequence variants of the antibodies provided herein are contemplated. For example, it may be desirable to alter the binding affinity and/or other biological properties of the antibody. In some embodiments, the antibody has been mutated to have a faster off rate compared to the parental antibody. In some embodiments, the antibody has a greater KD value compared to the parental antibody. In some embodiments, the antibody has a lesser affinity for the substrate. In some embodiments, the antibody has a weaker affinity for the target. In some embodiments, the antibody has both a faster off rate and a lesser affinity for the target. In some embodiments, the antibody has a lower KD value compared to the parental antibody. In some embodiments, the antibody has a stronger binding affinity for the target compared to the parental antibody.

Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

### (i) Substitution, Insertion, and Deletion Variants

In certain aspects, antibody variants having one or more amino acid substitutions are provided. Sites of interest for substitutional mutagenesis include the CDRs and FRs. Conservative substitutions are shown in Table 1 under the heading of “preferred substitutions”. More substantial changes are provided in Table 1 under the heading of “exemplary substitutions”, and as further described below in reference to amino acid side chain classes. Amino acid substitutions may be introduced into an antibody of interest and the products screened for a desired activity, e.g., retained/improved antigen binding, decreased immunogenicity, or improved ADCC or CDC.

TABLE 1

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gln;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for a member of another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more CDR residues are mutated and the variant antibodies displayed on phage and screened for a particular biological activity (e.g., binding affinity).

Alterations (e.g., substitutions) may be made in CDRs, e.g., to improve antibody affinity. Such alterations may be made in CDR “hotspots”, i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, NJ, (2001).) In some aspects of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted.

In certain aspects, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in the CDRs. Such alterations may, for example, be outside of antigen contacting residues in the CDRs. In certain variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex may be used to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT (antibody directed enzyme prodrug therapy)) or a polypeptide which increases the serum half-life of the antibody.

### (ii) Glycosylation Variants

In certain aspects, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the oligosaccharide attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some aspects, modifications of the oligosaccharide in an antibody of the invention may be made in order to create antibody variants with certain improved properties.

In one aspect, antibody variants are provided having a non-fucosylated oligosaccharide, i.e. an oligosaccharide structure that lacks fucose attached (directly or indirectly) to an Fc region. Such non-fucosylated oligosaccharide (also referred to as “afucosylated” oligosaccharide) particularly is an N-linked oligosaccharide which lacks a fucose residue attached to the first GlcNAc in the stem of the biantennary oligosaccharide structure. In one aspect, antibody variants are provided having an increased proportion of non-fucosylated oligosaccharides in the Fc region as compared to a native or parent antibody. For example, the proportion of non-fucosylated oligosaccharides may be at least about 20%, at least about 40%, at least about 60%, at least about 80%, or even about 100% (i.e. no fucosylated oligosaccharides are present). The percentage of non-fucosylated oligosaccharides is the (average) amount of oligosaccharides lacking fucose residues, relative to the sum of all oligosaccharides attached to Asn 297 (e. g. complex, hybrid and high mannose structures) as measured by MALDI-TOF mass spectrometry, as described in WO 2006/082515, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such antibodies having an increased proportion of non-fucosylated oligosaccharides in the Fc region may have improved FcγRIIIa receptor binding and/or improved effector function, in particular improved ADCC function. See, e.g., US 2003/0157108; US 2004/0093621.

Examples of cell lines capable of producing antibodies with reduced fucosylation include Lec13 CHO cells deficient in protein fucosylation (Ripka et al. Arch. Biochem. Biophys. 249:533-545 (1986); US 2003/0157108; and WO 2004/056312, especially at Example 11), and knockout cell lines, such as alpha-1,6-fucosyltransferase gene, FUT8, knockout CHO cells (see, e.g., Yamane-Ohnuki et al. Biotech. Bioeng. 87:614-622 (2004); Kanda, Y. et al., Biotechnol. Bioeng., 94(4):680-688 (2006); and WO 2003/085107), or cells with reduced or abolished activity of a GDP-fucose synthesis or transporter protein (see, e.g., US2004259150, US2005031613, US2004132140, US2004110282).

In a further aspect, antibody variants are provided with bisected oligosaccharides, e.g., in which a biantennary oligosaccharide attached to the Fc region of the antibody is bisected by GlcNAc. Such antibody variants may have reduced fucosylation and/or improved ADCC function as described above. Examples of such antibody variants are described, e.g., in Umana et al., Nat Biotechnol 17, 176-180 (1999); Ferrara et al., Biotechn Bioeng 93, 851-861 (2006); WO 99/54342; WO 2004/065540, WO 2003/011878.

Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

(iii) Fc Region Variants

In certain aspects, one or more amino acid modifications may be introduced into the Fc region of an antibody provided herein, thereby generating an Fc region variant. The Fc region variant may comprise a human Fc region sequence (e.g., a human IgG₁, IgG₂, IgG₃ or IgG₄ Fc region) comprising an amino acid modification (e.g., a substitution) at one or more amino acid positions.

In certain aspects, the invention contemplates an antibody variant that possesses some but not all effector functions, which make it a desirable candidate for applications in which the half life of the antibody in vivo is important yet certain effector functions (such as complement-dependent cytotoxicity (CDC) and antibody-dependent cell-mediated cytotoxicity (ADCC)) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express FcγRIII only, whereas monocytes express FcγRI, FcγRII and FcγRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l Acad. Sci. USA 83:7059-7063 (1986)) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, CA; and CytoTox 96® non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half life determinations can also be performed using methods known in the art (see, e.g., Petkova, S. B. et al., Int'l. Immunol. 18(12):1759-1769 (2006); WO 2013/120929 A1).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain aspects, an antibody variant comprises an Fc region with one or more amino acid substitutions which improve ADCC, e.g., substitutions at positions 298, 333, and/or 334 of the Fc region (EU numbering of residues).

In certain aspects, an antibody variant comprises an Fc region with one or more amino acid substitutions which diminish FcγR binding, e.g., substitutions at positions 234 and 235 of the Fc region (EU numbering of residues). In one aspect, the substitutions are L234A and L235A (LALA). In certain aspects, the antibody variant further comprises D265A and/or P329G in an Fc region derived from a human IgG₁ Fc region. In one aspect, the substitutions are L234A, L235A and P329G (LALA-PG) in an Fc region derived from a human IgG₁ Fc region. (See, e.g., WO 2012/130831). In another aspect, the substitutions are L234A, L235A and D265A (LALA-DA) in an Fc region derived from a human IgG₁ Fc region.

In some aspects, alterations are made in the Fc region that result in altered (e.g., diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934 (Hinton et al.). Those antibodies comprise an Fc region with one or more substitutions therein which improve binding of the Fc region to FcRn. Such Fc variants include those with substitutions at one or more of Fc region residues: 238, 252, 254, 256, 265, 272, 286, 303, 305, 307, 311, 312, 317, 340, 356, 360, 362, 376, 378, 380, 382, 413, 424 or 434, e.g., substitution of Fc region residue 434 (See, e.g., U.S. Pat. No. 7,371,826; Dall'Acqua, W. F., et al. J. Biol. Chem. 281 (2006) 23514-23524).

Fc region residues critical to the mouse Fc-mouse FcRn interaction have been identified by site-directed mutagenesis (see e.g. Dall'Acqua, W. F., et al. J. Immunol 169 (2002) 5171-5180). Residues I253, H310, H433, N434, and H435 (EU numbering of residues) are involved in the interaction (Medesan, C., et al., Eur. J. Immunol. 26 (1996) 2533; Firan, M., et al., Int. Immunol. 13 (2001) 993; Kim, J. K., et al., Eur. J. Immunol. 24 (1994) 542). Residues I253, H310, and H435 were found to be critical for the interaction of human Fc with murine FcRn (Kim, J. K., et al., Eur. J. Immunol. 29 (1999) 2819). Studies of the human Fc-human FcRn complex have shown that residues I253, S254, H435, and Y436 are crucial for the interaction (Firan, M., et al., Int. Immunol. 13 (2001) 993; Shields, R. L., et al., J. Biol. Chem. 276 (2001) 6591-6604). In Yeung, Y. A., et al. (J. Immunol. 182 (2009) 7667-7671) various mutants of residues 248 to 259 and 301 to 317 and 376 to 382 and 424 to 437 have been reported and examined.

In certain aspects, an antibody variant comprises an Fc region with one or more amino acid substitutions, which reduce FcRn binding, e.g., substitutions at positions 253, and/or 310, and/or 435 of the Fc-region (EU numbering of residues). In certain aspects, the antibody variant comprises an Fc region with the amino acid substitutions at positions 253, 310 and 435. In one aspect, the substitutions are I253A, H310A and H435A in an Fc region derived from a human IgG1 Fc-region. See, e.g., Grevys, A., et al., J. Immunol. 194 (2015) 5497-5508.

In certain aspects, an antibody variant comprises an Fc region with one or more amino acid substitutions, which reduce FcRn binding, e.g., substitutions at positions 310, and/or 433, and/or 436 of the Fc region (EU numbering of residues). In certain aspects, the antibody variant comprises an Fc region with the amino acid substitutions at positions 310, 433 and 436. In one aspect, the substitutions are H310A, H433A and Y436A in an Fc region derived from a human IgG1 Fc-region. (See, e.g., WO 2014/177460 Al).

In certain aspects, an antibody variant comprises an Fc region with one or more amino acid substitutions which increase FcRn binding, e.g., substitutions at positions 252, and/or 254, and/or 256 of the Fc region (EU numbering of residues). In certain aspects, the antibody variant comprises an Fc region with amino acid substitutions at positions 252, 254, and 256. In one aspect, the substitutions are M252Y, S254T and T256E in an Fc region derived from a human IgG₁ Fc-region. See also Duncan & Winter, Nature 322:738-40 (1988); U.S. Pat. Nos. 5,648,260; 5,624,821; and WO 94/29351 concerning other examples of Fc region variants.

In certain aspects, an antibody variant comprises an Fc region with one or more amino acid substitutions which decrease self-recognition, e.g., substitutions at positions R355, E356, K414, E438, K439, S440 of the Fc region (EU numbering of residues). In certain aspects, the antibody variant comprises an Fc region with amino acid substitutions at positions 252, 254, and 256. In one aspect, the substitutions are M252Y, S254T and T256E in an Fc region derived from a human IgG₁ Fc-region. See also Duncan & Winter, Nature 322:738-40 (1988); U.S. Pat. Nos. 5,648,260; 5,624,821; and WO 94/29351 concerning other examples of Fc region variants.

The C-terminus of the heavy chain of the antibody as reported herein can be a complete C-terminus ending with the amino acid residues PGK. The C-terminus of the heavy chain can be a shortened C-terminus in which one or two of the C terminal amino acid residues have been removed. In some aspects, the C-terminus of the heavy chain is a shortened C-terminus ending PG. In one aspect of all aspects as reported herein, an antibody comprising a heavy chain including a C-terminal CH3 domain as specified herein, comprises the C-terminal glycine-lysine dipeptide (G446 and K447, EU index numbering of amino acid positions). In one aspect of all aspects as reported herein, an antibody comprising a heavy chain including a C-terminal CH3 domain, as specified herein, comprises a C-terminal glycine residue (G446, EU index numbering of amino acid positions).

### (iv) Cysteine Engineered Antibody Variants

In certain aspects, it may be desirable to create cysteine engineered antibodies, e.g., THIOMAB™ antibodies, in which one or more residues of an antibody are substituted with cysteine residues. In particular aspects, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. Cysteine engineered antibodies may be generated as described, e.g., in U.S. Pat. Nos. 7,521,541, 830,930, 7,855,275, 9,000,130, or WO 2016040856.

### 8. Antibody Derivatives

In certain aspects, an antibody provided herein may be further modified to contain additional nonproteinaceous moieties that are known in the art and readily available. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

### Proteases

Proteases (proteinases) are catalytic enzymes that cleave polypeptides to make shorter polypeptides or single amino acids through hydrolysis, are biologically highly regulated, and are involved in several diseases (Bond, Journal of Biological Chemistry, Volume 294, Issue 5, 1643-1651). Proteolysis causes post translational processing of polypeptides. Proteases are varied and can be highly specific for a substrate, or nonspecific and cleave at a consensus sequence. Proteases are currently classified in seven broad groups: serine proteases, cysteine proteases, threonine proteases, aspartic proteases, glutamic proteases, metalloproteases, and asparagine peptide lyases. A list of proteases that are useful are identified in the MEROPS database (Rawlings, N. D., Barrett, A. J., Thomas, P. D., Huang, X., Bateman, A. & Finn, R. D. (2018) The MEROPS database of proteolytic enzymes, their substrates and inhibitors in 2017 and a comparison with peptidases in the PANTHER database. Nucleic Acids Res 46, D624-D632).

Proteases are an expanding class of drugs that hold great promise. See e.g., Biochem J. 2011 Apr. 1; 435(1): 1-16. The proteases provided herein cleaves a substrate, e.g., a substrate that involved in a disease or condition.

In some embodiments, the protease is a metalloprotease. In some embodiments, the protease is a zinc metalloprotease.

Proteases are found in many organisms, including of prokaryotic and eukaryotic origin. However, proteins of non-human origin may also be immunogenic in human systems, hindering repeat dosing and therefore limiting therapeutic application. In some aspects, the protease is of eukaryotic origin. In some aspects, the protease is of human origin. In some aspects, the protease is of prokaryotic origin. In some aspects, the protease is not of human origin.

Protease selection may also be guided by a combination of reasons such as specificity and/or potency against the desired substrate, yield, and immunogencity. Methods assessing these features include those described in the examples.

Protease selection may also be guided by the number of substrates that the protease may cleave. In some aspects, the protease has promiscuous activity. In some embodiments, the protease hydrolyzes a wide variety of peptide bonds. In some aspects, the protease has one substrate. In some aspects, the protease has one or more substrate. In some aspects, the protease does not have promiscuous activity. In some aspects, the protease hydrolyzes a limited or specific peptide bond. In some aspects, the protease is specific for a substrate or a class of substrates. In some aspects, the protease has one substrate. Proteases may be substrate isoform specific. Therefore, in some aspects, the protease recognizes one isoform. In some aspects, the protease prefers one isoform. Preference may be exhibited by binding, enhanced substrate engagement, enhanced catalysis, and other enzymatic and/or binding parameters that may be measured by biochemical assays, such as the assays described herein. In some aspects, the protease recognizes one or more isoform. In some aspects, the protease prefers one or more isoform. In some embodiments, the protease is not isoform specific. In some embodiments, the protease catalyzes hydrolysis of all isoforms equally well. In some embodiments, the protease catalyzes hydrolysis of one or more isoform better than other isoforms.

Protease selection may also be guided by the number of sites and/or the type of sequence that the protease may cleave. In some aspects, the protease recognizes a consensus sequence. In some aspects, the protease cleaves a consensus sequence. In some aspects, the protease recognizes a specific sequence. In some aspects, the protease cleaves a specific sequence.

Protease selection may also be guided by the rate of substrate catalysis. In some embodiments, the protease causes rapid hydrolysis of the substrate. In some aspects, the protease does not cause rapid hydrolysis of the substrate. In some aspects, the protease is engineered (e.g. mutated) to cause rapid hydrolysis of the substrate. In some aspects, the protease is engineered (e.g. mutated) to decrease the rate of hydrolysis of the substrate.

Proteases may also comprise other regulatory mechanisms that act as a switch to regulate catalytic activity. Other mechanisms regulating enzymatic activity include temperature (e.g. thermostable proteases), addition of organo-mercury or organic reagents, or partial proteolysis. A protease may comprise a pro-domain in addition to their catalytic domain. The pro-domain is a protein domain that regulates catalytic activity of the protease. For example, pro-domains may inhibit activity when conditions are not correct for protease function. In some aspects, successful expression of the protease for may require including pro-domain selection. In some aspects, the pro-domain is protelytically removed to induce activity.

Proteases selection may also be guided by functional assays. For example, the activity of several proteases may be tested on substrates (such as fluorogenic substrates). Candidate proteases may be elected based on comparative performance.

### Targets and Substrates

In some aspects, the targets to be bound by the antibodies described herein and the substrates to be cleaved by the proteases described herein are in proximity with each other such that the affinity/avidity of the antibodies against the targets promote the access of the substrate to the protease.

In some embodiments, the substrate is in the CNS. In some embodiments, the substrate is in the circulation. In some embodiments, the substrate is in a cancer tissue. In some embodiments, the substrate is in a fibrosis tissue. In some embodiments, the substrate is in a diseased tissue (e.g., with inflammation). In some embodiments, the substrate is in an extracellular tissue. In some embodiments, the level of the substrate is at least 5-fold, 10-fold, 20-fold, 50-fold, 100-fold, 1000-fold higher than a reference level of the substrate (e.g., the level of the substrate in a healthy individual in the same location). In some embodiments, the substrate is in circulation and has a concentration of at least about 0.1 mg/ml, 0.5 mg/ml, 1 ml/ml, 5 mg/ml, or 10 mg/ml.

In some embodiments, the target and the substrate are on adjacent cells. In some embodiments, the target and the substrate are on the same cell. In some embodiments, the target and the substrate are in the same complex (e.g., a macromolecular complex). In some embodiments, the target is a fragment or a portion of the substrate. In some embodiments, the substrate is a fragment or a portion of the target. In some embodiments, the target and the substrate are the same molecule (e.g. same polypeptide). In some embodiments, the antibody binds to an epitope of the target, and the epitope is not overlapping with where the protease binds to or cleaves the molecule. In some embodiments, the antibody binds to an epitope of the target, and the epitope is overlapping with where the protease binds to or cleaves the molecule.

In some embodiments, the target and the substrate is A-beta. In some embodiments, the antibody and/or the protease targets both Aβ₁₋₄₀ and Aβ₁₋₄₂. In some embodiments, the antibody is crenezumab or a variant thereof, or solanezumab or a variant thereof, optionally wherein the variant of crenezumb comprises G33S on its heavy chains, and further optionally wherein the variant of crenezumb comprises S56F on its light chains. In some embodiments, the protease is selected from the group consisting of neprolysin (NEP), neprolysin-2 (NEP2), endothelin-converting enzyme 1 and 2 (ECE1 and ECE2), angiotensin-converting enzyme (ACE), insulin-degrading enzyme (IDE), matrix metalloproteinase 2 and 9 (MMP2 and MMP9), and matriptase (MTSP1). In some embodiments, the protease is neprolysin.

In some embodiments, the target and the substrate is IgG. In some embodiments, the target and the substrate is an isotype of IgG. In some embodiments, the isotype is IgG₁, IgG₂, IgG₃ or IgG₄. In some embodiments, the protease is selected from the group consisting of matrix metalloproteinase 3 (MMP3), MMP7, Cathepsin G or a variant thereof. In some embodiments, the protease is MMP3 or a variant thereof. In some embodiments, the antibody comprises a full-length antibody, and optionally the antibody comprises a R335E mutation in the Fc Fragment. In some embodiments, the full-length antibody comprises a hinge region resistant to the protease, optionally wherein the hinge region comprises a (G₄A)₂ sequence. In some embodiments, the antibody comprises a Fab fragment. In some embodiments, the antibody comprises a Rheumatoid factor or a variant thereof.

### Fusion Protein Format/Structure

The antibodies and the proteases discussed herein can be fused in various ways to form fusion proteins.

In some embodiments, the antibody has one or more polypeptide chains, and the protease is fused to one or more polypeptide chains of the antibody.

In some embodiments, the protease is fused to the N-terminus and/or C-terminus of one or more polypeptide chains of the antibody.

In some embodiments, the antibody comprises:
a) a heavy chain variable domain (VH) and a light chain variable domain (VL), wherein the protease is fused to the N-terminus or C-terminus of the VH or VL; or
b) a full-length antibody comprising two heavy chains and two light chains, wherein the protease is fused to the N-terminus or C-terminus of one or both of the two heavy chains and/or the two light chains.

In some embodiments, the antibody comprises a) a first polypeptide comprising a heavy chain variable (VH) domain and a first heavy chain constant (CH1) domain and b) a second polypeptide comprising a light chain variable (VL) domain and a light chain constant (CL) domain, wherein the protease is fused to the N-terminus of the VL domain, wherein the VH domain and VL domain form a binding domain for the target, wherein a disulfide bond is formed between the CH1 domain and the CL domain. In some embodiments, the antibody does not comprise a Fc fragment.

In some embodiments, the antibody comprises a) a first polypeptide comprising a heavy chain variable (VH) domain and a first heavy chain constant (CH1) domain and b) a second polypeptide comprising a light chain variable (VL) domain and a light chain constant (CL) domain, wherein the protease is fused to the N-terminus of the VH domain, wherein the VH domain and VL domain form a binding domain for the target, wherein a disulfide bond is formed between the CH1 domain and the CL domain. In some embodiments, the antibody does not comprise a Fc fragment.

In some embodiments, the antibody comprises a) a first polypeptide comprising a light chain variable (VL) domain and a light chain constant (CL) domain, b) a second polypeptide comprising a heavy chain variable (VH) domain and first heavy chain constant (CH1) domains, and c) a third polypeptide comprising a Fc region, wherein the protease is fused to the N-terminus of the third polypeptide, and wherein the VH domain and the VL domain form a binding domain for the target.

In some embodiments, the antibody comprises two heavy chains and two light chains, wherein each heavy chain comprises a heavy chain variable (VH) domain and a heavy chain constant (CH) domain, wherein each light chain comprises a light chain variable (VL) domain and a light chain constant (CL) domain, wherein the protease is fused to N-terminus of one or both of light chains.

In some embodiments, the antibody comprises two heavy chains and two light chains, wherein each heavy chain comprises a heavy chain variable (VH) domain and a heavy chain constant (CH) domain, wherein each light chain comprises a light chain variable (VL) domain and a light chain constant (CL) domain, wherein the protease is fused to N-terminus of one or both of heavy chains.

In some embodiments, the antibody comprises two heavy chains and two light chains, wherein each heavy chain comprises a heavy chain variable (VH) domain and a heavy chain constant (CH) domain, wherein each light chain comprises a light chain variable (VL) domain and a light chain constant (CL) domain, wherein the protease is fused to N-terminus of both of light chains and N-terminus of both of the heavy chains.

In some embodiments, the antibody comprises two heavy chains and two light chains, wherein each heavy chain comprises a heavy chain variable (VH) domain and a heavy chain constant (CH) domain, wherein each light chain comprises a light chain variable (VL) domain and a light chain constant (CL) domain, wherein the protease is fused to C-terminus of one of the heavy chains.

In some embodiments, the antibody comprises two heavy chains and two light chains, wherein each heavy chain comprises a heavy chain variable (VH) domain and a heavy chain constant (CH) domain, wherein each light chain comprises a light chain variable (VL) domain and a light chain constant (CL) domain, wherein the protease is fused to C-terminus of both of the heavy chains.

Considerations for identifying an optimal geometry for the fusion proteins may be evaluated through one or more parameters. See e.g., examples in the present application. In some aspects, the geometry of the fusion protein is determined e.g., in part by in vitro expression and purification. In some aspects, the geometry is determined e.g., in part by protein yield. In some aspects, the geometry is determined e.g., in part by target (e.g. substrate) binding and/or recognition. In some aspects, the geometry is determined e.g., in part by measured or observed activity, such as in a biochemical assay. In some aspects, the geometry of the fusion protein is determined e.g., by the accessibility of the protease cleavage site. In some embodiments, the antibody epitope and the protease cleavage site are not the same. In some aspects, the fusion protein is engineered with one protease per molecule. In some aspects, the fusion protein is engineered with two or more proteases (e.g., two, three, or four) per molecule.

### Linkers

In some embodiments, the antibodies and the proteases are fused to each other via a linker.

The length, the degree of flexibility and/or other properties of the linker used in the fusion proteins described herein may have some influence on properties, including but not limited to the affinity, specificity or avidity of antibody or protease, and/or affinity, specificity or avidity for one or more particular antigens or epitopes present on target or substrate. For example, longer linkers may be selected to ensure that the antibody moiety and the protease moiety do not sterically interfere with one another. In some embodiments, a linker (such as peptide linker) comprises flexible residues (such as glycine and serine) so that the adjacent moieties are free to move relative to each other. For example, a glycine-serine doublet can be a suitable peptide linker. In some embodiments, the linker is a non-peptide linker. In some embodiments, the linker is a peptide linker. In some embodiments, the linker is a non-cleavable linker. In some embodiments, the linker is a cleavable linker (but not cleavable by the protease in the fusion protein).

Other linker considerations include the effect on physical or pharmacokinetic properties of the resulting fusion protein, such as solubility, lipophilicity, hydrophilicity, hydrophobicity, stability (more or less stable as well as planned degradation), rigidity, flexibility, immunogenicity, modulation of antibody binding, the ability to be incorporated into a micelle or liposome, and the like.

a. Non-Peptide Linkers

Any one or all of the linkers described herein can be accomplished by any chemical reaction that will bind the two molecules so long as the components or fragments retain their respective activities. This linkage can include many chemical mechanisms, for instance covalent binding, affinity binding, intercalation, coordinate binding and complexation. In some embodiments, the binding is covalent binding. Covalent binding can be achieved either by direct condensation of existing side chains or by the incorporation of external bridging molecules. Many bivalent or polyvalent linking agents are useful in coupling protein molecules, such as an Fc fragment to the antibody of the present invention. For example, representative coupling agents can include organic compounds such as thioesters, carbodiimides, succinimide esters, diisocyanates, glutaraldehyde, diazobenzenes and hexamethylene diamines. This listing is not intended to be exhaustive of the various classes of coupling agents known in the art but, rather, is exemplary of the more common coupling agents (see Killen and Lindstrom, Jour. Immun. 133:1335-2549 (1984); Jansen et al., Immunological Reviews 62:185-216 (1982); and Vitetta et al., Science 238:1098 (1987), each incorporated by reference in their entirety for all purposes).

Linkers that can be applied in the present application are described in the literature (see, for example, Ramakrishnan, S. et al., Cancer Res. 44:201-208 (1984) describing use of MBS (M-maleimidobenzoyl-N-hydroxysuccinimide ester), incorporated by reference in its entirety for all purposes). In some embodiments, non-peptide linkers used herein include: (i) EDC (1-ethyl-3-(3-dimethylamino-propyl) carbodiimide hydrochloride; (ii) SMPT (4-succinimidyloxycarbonyl-alpha-methyl-alpha-(2-pridyl-dithio)-toluene (Pierce Chem. Co., Cat. (21558G); (iii) SPDP (succinimidyl-6 [3-(2-pyridyldithio) propionamido]hexanoate (Pierce Chem. Co., Cat #21651G); (iv) Sulfo-LC-SPDP (sulfosuccinimidyl 6 [3-(2-pyridyldithio)-propianamide]hexanoate (Pierce Chem. Co. Cat. #2165-G); and (v) sulfo-NHS (N-hydroxysulfo-succinimide: Pierce Chem. Co., Cat. #24510) conjugated to EDC.

The linkers described above contain components that have different attributes, thus leading to fusion proteins with differing physio-chemical properties. For example, sulfo-NHS esters of alkyl carboxylates are more stable than sulfo-NHS esters of aromatic carboxylates. NHS-ester containing linkers are less soluble than sulfo-NHS esters. Further, the linker SMPT contains a sterically hindered disulfide bond, and can form fusion protein with increased stability. Disulfide linkages, are in general, less stable than other linkages because the disulfide linkage is cleaved in vitro, resulting in less fusion protein available. Sulfo-NHS, in particular, can enhance the stability of carbodimide couplings. Carbodimide couplings (such as EDC) when used in conjunction with sulfo-NHS, forms esters that are more resistant to hydrolysis than the carbodimide coupling reaction alone.

b. Peptide Linkers

Any one or all of the linkers described herein can be peptide linkers. The peptide linker may have a naturally occurring sequence, or a non-naturally occurring sequence. For example, a sequence derived from the hinge region of heavy chain only antibodies may be used as the linker. See, for example, WO1996/34103, incorporated by reference in its entirety for all purposes. In some embodiments, the peptide linker comprises the amino acid sequence of CPPCP, a sequence found in the native IgG1 hinge region.

The peptide linker can be of any suitable length. In some embodiments, the length of the peptide linker is any of about 1 aa to about 10 aa, about 1 aa to about 20 aa, about 1 aa to about 30 aa, about 5 aa to about 15 aa, about 10 aa to about 25 aa, about 5 aa to about 30 aa, about 10 aa to about 30 aa, about 30 aa to about 50 aa, about 50 aa to about 100 aa, or about 1 aa to about 100 aa.

An essential technical feature of such peptide linker is that said peptide linker does not comprise any polymerization activity. The characteristics of a peptide linker, which comprise the absence of the promotion of secondary structures, are known in the art and described, e.g., in Dall'Acqua et al. (Biochem. (1998) 37, 9266-9273), Cheadle et al. (Mol Immunol (1992) 29, 21-30) and Raag and Whitlow (FASEB (1995) 9(1), 73-80, each incorporated by reference in their entirety for all purposes). A particularly preferred amino acid in context of the “peptide linker” is Gly. Furthermore, peptide linkers that also do not promote any secondary structures are preferred. The linkage of the molecules to each other can be provided by, e.g., genetic engineering. Methods for preparing fused and operatively linked antibody constructs and expressing them in mammalian cells or bacteria are well-known in the art (e.g. WO 99/54440, Ausubel, Current Protocols in Molecular Biology, Green Publishing Associates and Wiley Interscience, N. Y. 1989 and 1994 or Sambrook et al., Molecular Cloning: A Laboratory Manual, Cold Spring Harbor Laboratory Press, Cold Spring Harbor, N. Y., 2001, each incorporated by reference in their entirety for all purposes).

In some embodiments, the peptide linker is a stable linker, which is not cleavable by protease, such as by Matrix metalloproteinases (MMPs).

In some embodiments, the peptide linker tends not to adopt a rigid three-dimensional structure, but rather provide flexibility to a polypeptide (e.g., first and/or second components), such as providing flexibility between the antibody and the protease. In some embodiments, the peptide linker is a flexible linker. Exemplary flexible linkers include glycine polymers (G)ₙ, glycine-serine polymers (including, for example, (GS)ₙ, (GSGGS)ₙ (SEQ ID NO. 7), (GGGGS)ₙ (SEQ ID NO. 2), and (GGGS)ₙ (SEQ ID NO. 8), where n is an integer of at least one), glycine-alanine polymers, alanine-serine polymers, and other flexible linkers known in the art. Glycine and glycine-serine polymers are relatively unstructured, and therefore may be able to serve as a neutral tether between components. Glycine accesses significantly more phi-psi space than even alanine, and is much less restricted than residues with longer side chains (see Scheraga, Rev. Computational Chem. 11 173-142 (1992)). The ordinarily skilled artisan will recognize that design of a fusion protein can include linkers that are all or partially flexible, such that the linker can include a flexible linker portion as well as one or more portions that confer less flexible structure to provide a desired fusion protein structure.

In some embodiments, the antibody and the substrate are linked together by a linker of sufficient length to enable the fusion protein to fold in such a way as to permit binding to the target and cleaving the substrate. In some embodiments, the linker is or comprises a (GGGGS)ₙ (SEQ ID NO. 2) sequence, wherein n is equal to 1, 2, 3, 4, 5, 6, 7, 8, 9, 10 or more.

Natural linkers adopt various conformations in secondary structure, such as helical, β-strand, coil/bend and turns, to exert their functions. Linkers in an α-helix structure might serve as rigid spacers to effectively separate protein domains, thus reducing their unfavorable interactions. Non-helical linkers with Pro-rich sequence could increase the linker rigidity and function in reducing inter-domain interference.

In some embodiments, the protease is fused with the antibody directly without a linker. In some embodiments, the protease is fused with the antibody via a linker. In some embodiments, the linker is a peptide linker. In some embodiments, the linker has a length of at least about 5, 10, 15, 20, 25, or 30 amino acids. In some embodiments, the linker is at least about 5 amino acids. In some embodiments, the linker is at least about 10 amino acids.

In some embodiments, the linker is cleavable by a protease distinct from the protease comprised in the fusion protein. In some embodiments, the linker is not cleavable. In some embodiments, the linker is not cleavable by the protease in the fusion protein.

In some embodiments, the linker is a proline-rich linker. In some embodiments, the linker is a GGGGA (SEQ ID NO. 3) linker, or variant thereof. In some embodiments, the linker is a (G₄A)₂ (SEQ ID NO. 4) linker.

Linker placement may be identified based on evaluation of optimal geometry as disclosed herein. In some embodiments, the linker is placed following the hinge disulfides (e.g. the lower hinge and N-terminal region of the CH2 domain of the antibody).

Linker placement and linker length of the fusion protein may also be varied to influence substrate capture and/or enzymatic cleavage. These parameters may be assessed, for example, by biochemical assays such as the assays disclosed herein.

### B. Recombinant Methods and Compositions

Fusion proteins may be produced using recombinant methods and compositions, e.g., as described in the examples. For these methods one or more isolated nucleic acid(s) encoding a fusion protein are provided. When more than one isolated nucleic acids were used, these nucleic acids can be on the same expression vector or on different expression vectors, normally these nucleic acids are located on two or three expression vectors, i.e. one vector can comprise more than one of these nucleic acids. Examples of these bispecific antibodies are CrossMabs (see, e.g., Schaefer, W. et al, PNAS, 108 (2011) 11187-1191). For example, one of the heteromonomeric heavy chain comprises the so-called “knob mutations” (T366W and optionally one of S354C or Y349C) and the other comprises the so-called “hole mutations” (T366S, L368A and Y407V and optionally Y349C or S354C) (see, e.g., Carter, P. et al., Immunotechnol. 2 (1996) 73) according to EU index numbering.

In one aspect, isolated nucleic acids encoding a fusion protein as reported herein are provided.

In one aspect, a method of making a fusion protein described herein is provided, wherein the method comprises culturing a host cell comprising nucleic acid(s) encoding the fusion protein, as provided above, under conditions suitable for expression of the fusion protein, and optionally recovering the fusion protein from the host cell (or host cell culture medium).

For recombinant production of a fusion protein, nucleic acids encoding the fusion protein, e.g., as described above, are isolated and inserted into one or more vectors for further cloning and/or expression in a host cell. Such nucleic acids may be readily isolated and sequenced using conventional procedures (e.g., by using oligonucleotide probes that are capable of binding specifically to genes encoding the heavy and light chains of the antibody) or produced by recombinant methods or obtained by chemical synthesis.

Suitable host cells for cloning or expression of fusion protein-encoding vectors include prokaryotic or eukaryotic cells described herein. For example, fusion proteins may be produced in bacteria, in particular when glycosylation and Fc effector function are not needed. For expression of fusion proteins and polypeptides in bacteria, see, e.g., U.S. Pat. Nos. 5,648,237, 5,789,199, and 5,840,523. (See also Charlton, K. A., In: Methods in Molecular Biology, Vol. 248, Lo, B. K. C. (ed.), Humana Press, Totowa, NJ (2003), pp. 245-254, describing expression of antibody fragments in E. coli.) After expression, the fusion protein may be isolated from the bacterial cell paste in a soluble fraction and can be further purified.

In addition to prokaryotes, eukaryotic microbes such as filamentous fungi or yeast are suitable cloning or expression hosts for fusion protein-encoding vectors, including fungi and yeast strains whose glycosylation pathways have been “humanized”, resulting in the production of a fusion protein with a partially or fully human glycosylation pattern. See Gerngross, T. U., Nat. Biotech. 22 (2004) 1409-1414; and Li, H. et al., Nat. Biotech. 24 (2006) 210-215.

Suitable host cells for the expression of (glycosylated) fusion protein are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham, F. L. et al., J. Gen Virol. 36 (1977) 59-74); baby hamster kidney cells (BHK); mouse sertoli cells (TM4 cells as described, e.g., in Mather, J. P., Biol. Reprod. 23 (1980) 243-252); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells (as described, e.g., in Mather, J. P. et al., Annals N.Y. Acad. Sci. 383 (1982) 44-68); MRC 5 cells; and FS4 cells. Other useful mammalian host cell lines include Chinese hamster ovary (CHO) cells, including DHFR-CHO cells (Urlaub, G. et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. and Wu, A. M., Methods in Molecular Biology, Vol. 248, Lo, B. K. C. (ed.), Humana Press, Totowa, NJ (2004), pp. 255-268.

In one aspect, the host cell is eukaryotic, e.g., a Chinese Hamster Ovary (CHO) cell or lymphoid cell (e.g., Y0, NS0, Sp20 cell).

### C. Assays

Fusion proteins provided herein may be identified, screened for, or characterized for their physical/chemical properties and/or biological activities by various assays known in the art.

### 1. Binding Assays and Other Assays for Antibodies in the Fusion Proteins

In one aspect, an antibody in the fusion protein is tested for its antigen binding activity, e.g., by known methods such as ELISA, Western blot, etc.

In another aspect, competition assays may be used to identify an antibody that competes with a reference antibody for binding to a desired target. In certain aspects, such a competing antibody binds to the same epitope (e.g., a linear or a conformational epitope) that is bound by the reference antibody. Detailed exemplary methods for mapping an epitope to which an antibody binds are provided in Morris (1996) “Epitope Mapping Protocols”, in Methods in Molecular Biology vol. 66 (Humana Press, Totowa, NJ).

In an exemplary competition assay, immobilized target is incubated in a solution comprising a first labeled antibody that binds to target (e.g., reference antibody) and a second unlabeled antibody that is being tested for its ability to compete with the first antibody for binding to the target. The second antibody may be present in a hybridoma supernatant. As a control, immobilized target is incubated in a solution comprising the first labeled antibody but not the second unlabeled antibody. After incubation under conditions permissive for binding of the first antibody to target, excess unbound antibody is removed, and the amount of label associated with immobilized target is measured. If the amount of label associated with immobilized target is substantially reduced in the test sample relative to the control sample, then that indicates that the second antibody is competing with the first antibody for binding to target. See Harlow and Lane (1988) Antibodies: A Laboratory Manual ch. 14 (Cold Spring Harbor Laboratory, Cold Spring Harbor, NY).

### 2. Activity Assays

In one aspect, assays are provided for identifying antibodies or proteases thereof having biological activity. Biological activity may include, e.g., proteases cleaving the substrate. Proteases having such biological activity in vivo and/or in vitro are also provided.

In certain aspects, a fusion protein described herein is tested for such biological activity.

### D. Pharmaceutical Compositions

In a further aspect, provided are pharmaceutical compositions comprising any of the fusion proteins provided herein, e.g., for use in any of the below therapeutic methods. In one aspect, a pharmaceutical composition comprises any of the fusion proteins provided herein and a pharmaceutically acceptable carrier. In another aspect, a pharmaceutical composition comprises any of the fusion proteins provided herein and at least one additional therapeutic agent, e.g., as described below.

Pharmaceutical compositions (formulations) of a fusion protein as described herein can be prepared by combining the fusion protein with pharmaceutically acceptable carriers or excipients known to the skilled person. See, for example, WO2019/224842, Remington's Pharmaceutical Sciences 16th edition, Osol, A. Ed. (1980), Shire S., Monoclonal Antibodies: Meeting the Challenges in Manufacturing, Formulation, Delivery and Stability of Final Drug Product, 1st Ed., Woodhead Publishing (2015), § 4 and Falconer R. J., Biotechnology Advances (2019), 37, 107412. Exemplary pharmaceutical compositions of a fusion protein as described herein are lyophilized, aqueous, frozen, etc.

Pharmaceutically acceptable carriers are generally nontoxic to recipients at the dosages and concentrations employed, and include, but are not limited to: buffers such as histidine, phosphate, citrate, acetate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG).

The pharmaceutical composition herein may also contain more than one active ingredients as necessary for the particular indication being treated, preferably those with complementary activities that do not adversely affect each other. Such active ingredients are suitably present in combination in amounts that are effective for the purpose intended.

The pharmaceutical compositions to be used for in vivo administration are generally sterile. Sterility may be readily accomplished, e.g., by filtration through sterile filtration membranes.

### E. Therapeutic Methods and Routes of Administration

Any of the fusion proteins provided herein may be used in therapeutic methods.

In one aspect, a fusion protein for use as a medicament is provided. In further aspects, a fusion protein for use in treating a disease or condition is provided. In certain aspects, a fusion protein for use in a method of treatment is provided. In certain aspects, the invention provides a fusion protein for use in a method of treating an individual having a disease or condition (e.g., a disease or condition that involves a substrate) comprising administering to the individual an effective amount of the fusion protein. In one such aspect, the method further comprises administering to the individual an effective amount of at least one additional therapeutic agent (e.g., one, two, three, four, five, or six additional therapeutic agents), e.g., as described below. In further aspects, the invention provides a fusion protein for use in e.g., promoting specificity/potency of the protease against the substrate, facilitating access of the protease to the substrate. In certain aspects, the invention provides a fusion protein for use in a method of promoting specificity/potency of the protease against the substrate, facilitating access of the protease to the substrate in an individual comprising administering to the individual an effective amount of the fusion protein. An “individual” according to any of the above aspects is preferably a human.

In a further aspect, the invention provides for the use of fusion protein in the manufacture or preparation of a medicament. In one aspect, the medicament is for treatment of a disease or condition that involves or is caused by abnormal deposition of a substrate. In a further aspect, the medicament is for use in a method of treating a disease comprising administering to an individual having a disease an effective amount of the medicament. In one such aspect, the method further comprises administering to the individual an effective amount of at least one additional therapeutic agent, e.g., as described below. In a further aspect, the medicament is for use in a method of promoting specificity/potency of the protease against the substrate and/or facilitating access of the protease to the substrate in an individual comprising administering to the individual an effective amount of the medicament. An “individual” according to any of the above aspects may be a human.

In a further aspect, the invention provides a method for treating a disease or condition (e.g., a disease or condition that involves or is caused by abnormal deposition of a substrate). In one aspect, the method comprises administering to an individual having such disease or condition an effective amount of a fusion protein. In one such aspect, the method further comprises administering to the individual an effective amount of at least one additional therapeutic agent, as described below.

An “individual” according to any of the above aspects may be a human.

In a further aspect, the invention provides a method for promoting specificity/potency of the protease against the substrate and/or facilitating access of the protease to the substrate in an individual. In one aspect, the method comprises administering to the individual an effective amount of a fusion protein. In one aspect, an “individual” is a human.

In a further aspect, the invention provides pharmaceutical compositions comprising any of the fusion proteins provided herein, e.g., for use in any of the above therapeutic methods. In one aspect, a pharmaceutical composition comprises any of the fusion proteins provided herein and a pharmaceutically acceptable carrier. In another aspect, a pharmaceutical composition comprises any of the fusion proteins provided herein and at least one additional therapeutic agent, e.g., as described below.

Antibodies of the invention can be administered alone or used in a combination therapy. For instance, the combination therapy includes administering a fusion protein of the invention and administering at least one additional therapeutic agent (e.g. one, two, three, four, five, or six additional therapeutic agents). In certain aspects, the combination therapy comprises administering a fusion protein of the invention and administering at least one additional therapeutic agent.

Such combination therapies noted above encompass combined administration (where two or more therapeutic agents are included in the same or separate pharmaceutical compositions), and separate administration, in which case, administration of the antibody of the invention can occur prior to, simultaneously, and/or following, administration of the additional therapeutic agent or agents. In one aspect, administration of the fusion protein and administration of an additional therapeutic agent occur within about one month, or within about one, two or three weeks, or within about one, two, three, four, five, or six days, of each other. In one aspect, the antibody and additional therapeutic agent are administered to the patient on Day 1 of the treatment. Fusion proteins of the invention can also be used in combination with radiation therapy.

A fusion protein of the invention (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

Fusion proteins of the invention would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The fusion protein need not be, but is optionally formulated with one or more agents currently used to prevent or treat the disorder in question. The effective amount of such other agents depends on the amount of antibody present in the pharmaceutical composition, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention or treatment of disease, the appropriate dosage of a fusion protein of the invention (when used alone or in combination with one or more other additional therapeutic agents) will depend on the type of disease to be treated, the type of antibody, the severity and course of the disease, whether the fusion protein is administered for preventive or therapeutic purposes, previous therapy, the patient's clinical history and response to the fusion protein, and the discretion of the attending physician. The fusion protein is suitably administered to the patient at one time or over a series of treatments. For repeated administrations over several days or longer, depending on the condition, the treatment would generally be sustained until a desired suppression of disease symptoms occurs. However, other dosage regimens may be useful. The progress of this therapy is easily monitored by conventional techniques and assays.

### F. Articles of Manufacture

In another aspect of the invention, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the invention. The label or package insert indicates that the composition is used for treating the condition of choice. Moreover, the article of manufacture may comprise (a) a first container with a composition contained therein, wherein the composition comprises an antibody of the invention; and (b) a second container with a composition contained therein, wherein the composition comprises a further cytotoxic or otherwise therapeutic agent. The article of manufacture in this aspect of the invention may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

SEQUENCE LISTING

| SEQ | | |
|---|---|---|
| ID | | |
| NO | SEQ | Description |
| 1 | MGWSCIILFLVATATGVHS | N-terminal secretion signal |
| 2 | (GGGGS)n | Linker |
| 3 | (GGGGA)n | Linker; n = any integer |
| 4 | GGGGA | Linker n = 2 |
| 5 | DDDDK | enterokinase |
| 6 | IEGR | Factor Xa cleavage site |
| 7 | (GSGGS)n | Linker; n = any integer |
| 8 | (GGGS)n | Linker; n = any integer |
| 9 | | |
| 10 | ATGGGATGGTCATGTATCATCCTTTTTCTAGTAGCAACTGCA | IgG-CTF-2 format, |
| | ACCGGTGTACATTCAGAAGTTCAGCTGGTGGAGTCTGGCGGT | neprolysin, anti-gD, heavy |
| | GGCCTGGTGCAGCCAGGGGGCTCACTCCGTTTGTCCTGTGCC | chain |
| | GCTTCTGGCTACTCCATCACCTCCGACTTTGCCTGGAACTGG | |
| | GTCCGTCAGGCCCCGGGTAAGGGCCTGGAATGGGTTGGATA | |
| | CATTAGTTACTCTGGAACCACTAGCTATAACCCTAGCCTGAA | |
| | GTCCCGTATCACTATAAGTCGCGACAATTCCAAAAACACATT | |
| | CTACCTGCAGATGAACAGCCTGCGTGCTGAGGACACTGCCGT | |
| | CTATTATTGTGCTCGAGAAAACTACTATGGCCGTTCTCACGTT | |
| | GGGTACTTCGACGTCTGGGGTCAAGGAACCCTGGTCACCGTC | |
| | TCGAGTGCCTCCACCAAGGGCCCATCGGTCTTCCCCCTGGCA | |
| | CCCTCCTCCAAGAGCACCTCTGGGGGCACAGCGGCCCTGGGC | |
| | TGCCTGGTCAAGGACTACTTCCCCGAGCCGGTGACGGTGTCG | |
| | TGGAACTCAGGCGCCCTGACCAGCGGCGTGCACACCTTCCCG | |
| | GCTGTCCTACAGTCCTCAGGACTCTACTCCCTCAGCAGCGTG | |
| | GTGACTGTGCCCTCTAGCAGCTTGGGCACCCAGACCTACATC | |
| | TGCAACGTGAATCACAAGCCCAGCAACACCAAGGTGGACAA | |
| | GAAAGTTGAGCCCAAATCTTGTGACAAAACTCACACATGCCC | |
| | ACCGTGCCCAGCACCTGAACTCCTGGGGGGACCGTCAGTCTT | |
| | CCTCTTCCCCCCAAAACCCAAGGACACCCTCATGATCTCCCG | |
| | GACCCCTGAGGTCACATGCGTGGTGGTGGACGTGAGCCACG | |
| | AAGACCCTGAGGTCAAGTTCAACTGGTACGTGGACGGCGTG | |
| | GAGGTGCATAATGCCAAGACAAAGCCGCGGGAGGAGCAGTA | |
| | CAACAGCACGTACCGTGTGGTCAGCGTCCTCACCGTCCTGCA | |
| | CCAGGACTGGCTGAATGGCAAGGAGTACAAGTGCAAGGTCT | |
| | CCAACAAAGCCCTCCCAGCCCCCATCGAGAAAACCATCTCCA | |
| | AAGCCAAAGGGCAGCCCCGAGAACCACAGGTGTACACCCTG | |
| | CCCCCATCCCGGGAAGAGATGACCAAGAACCAGGTCAGCCT | |
| | GACCTGCCTGGTCAAAGGCTTCTATCCCAGCGACATCGCCGT | |
| | GGAGTGGGAGAGCAATGGGCAGCCGGAGAACAACTACAAG | |
| | ACCACGCCTCCCGTGCTGGACTCCGACGGCTCCTTCTTCCTCT | |
| | ACAGCAAGCTCACCGTGGACAAGAGCAGGTGGCAGCAGGGG | |
| | AACGTCTTCTCATGCTCCGTGATGCATGAGGCTCTGCACAAC | |
| | CACTACACGCAGAAGAGCCTCTCCCTGTCTCCGGGTGGCGGC | |
| | GGCGGATCCTACGACGACGGCATCTGCAAGAGCAGCGACTG | |
| | CATCAAGAGCGCCGCCAGACTGATCCAGAACATGGACGCCA | |
| | CCACCGAGCCCTGCACCGACTTCTTCAAGTACGCCTGCGGCG | |
| | GCTGGCTGAAGAGAAACGTGATCCCCGAGACCAGCAGCAGA | |
| | TACGGCAACTTCGACATCCTGAGAGACGAGCTGGAGGTGGT | |
| | GCTGAAGGATGTGCTGCAGGAGCCCAAGACCGAGGACATCG | |
| | TGGCCGTGCAGAAGGCCAAGGCCCTGTACAGAAGCTGCATC | |
| | AACGAGAGCGCCATCGACAGCAGAGGCGGCGAGCCCCTGCT | |
| | GAAGCTGCTGCCCGACATCTACGGCTGGCCCGTGGCCACCGA | |
| | GAACTGGGAGCAGAAGTACGGCGCCAGCTGGACCGCCGAGA | |
| | AGGCCATCGCCCAGCTGAACAGCAAGTACGGCAAGAAGGTG | |
| | CTGATCAACCTGTTCGTGGGCACCGACGACAAGAACAGCGT | |
| | GAACCACGTGATCCACATCGACCAGCCCAGACTGGGCCTGCC | |
| | CAGCAGAGACTACTACGAGTGCACCGGCATCTACAAGGAGG | |
| | CCTGCACCGCCTACGTGGACTTCATGATCAGCGTGGCCAGAC | |
| | TGATCAGACAGGAGGAGAGACTGCCCATCGACGAGAACCAG | |
| | CTGGCCCTGGAGATGAACAAGGTGATGGAGCTGGAGAAGGA | |
| | GATCGCCAACGCCACCGCCAAGCCCGAGGACAGAAACGACC | |
| | CCATGCTGCTGTACAACAAGATGACCCTGGCCCAGATCCAGA | |
| | ACAACTTCAGCCTGGAGATCAACGGCAAGCCCTTCAGCTGGC | |
| | TGAACTTCACCAACGAGATCATGAGCACCGTGAACATCAGC | |
| | ATCACCAACGAGGAGGATGTGGTGGTGTACGCCCCCGAGTA | |
| | CCTGACCAAGCTGAAGCCCATCCTGACCAAGTACAGCGCCA | |
| | GAGACCTGCAGAACCTGATGAGCTGGAGATTCATCATGGAC | |
| | CTGGTGAGCAGCCTGAGCAGAACCTACAAGGAGAGCAGAAA | |
| | CGCCTTCAGAAAGGCCCTGTACGGCACCACCAGCGAGACCG | |
| | CCACCTGGAGAAGATGCGCCAACTACGTGAACGGCAACATG | |
| | GAGAACGCCGTGGGCAGACTGTACGTGGAGGCCGCCTTCGC | |
| | CGGCGAGAGCAAGCACGTGGTGGAGGACCTGATCGCCCAGA | |
| | TCAGAGAGGTGTTCATCCAGACCCTGGACGACCTGACCTGGA | |
| | TGGACGCCGAGACCAAGAAGAGAGCCGAGGAGAAGGCCCTG | |
| | GCCATCAAGGAGAGAATCGGCTACCCCGACGACATCGTGAG | |
| | CAACGACAACAAGCTGAACAACGAGTACCTGGAGCTGAACT | |
| | ACAAGGAGGACGAGTACTTCGAGAACATCATCCAGAACCTG | |
| | AAGTTCAGCCAGAGCAAGCAGCTGAAGAAGCTGAGAGAGAA | |
| | GGTGGACAAGGACGAGTGGATCAGCGGCGCCGCCGTGGTGA | |
| | ACGCCTTCTACAGCAGCGGCAGAAACCAGATCGTGTTCCCCG | |
| | CCGGCATCCTGCAGCCCCCCTTCTTCAGCGCCCAGCAGAGCA | |
| | ACAGCCTGAACTACGGCGGCATCGGCATGGTGATCGGCCAC | |
| | GAGATCACCCACGGCTTCGACGACAACGGCAGAAACTTCAA | |
| | CAAGGACGGCGACCTGGTGGACTGGTGGACCCAGCAGAGCG | |
| | CCAGCAACTTCAAGGAGCAGAGCCAGTGCATGGTGTACCAG | |
| | TACGGCAACTTCAGCTGGGACCTGGCCGGCGGCCAGCACCTG | |
| | AACGGCATCAACACCCTGGGCGAGAACATCGCCGACAACGG | |
| | CGGCCTGGGCCAGGCCTACAGAGCCTACCAGAACTACATCA | |
| | AGAAGAACGGCGAGGAGAAGCTGCTGCCCGGCCTGGACCTG | |
| | AACCACAAGCAGCTGTTCTTCCTGAACTTCGCCCAGGTGTGG | |
| | TGCGGCACCTACAGACCCGAGTACGCCGTGAACAGCATCAA | |
| | GACCGATGTGCACAGCCCCGGCAACTTCAGAATCATCGGCAC | |
| | CCTGCAGAACAGCGCCGAGTTCAGCGAGGCCTTCCACTGCAG | |
| | AAAGAACAGCTACATGAACCCCGAGAAGAAGTGCAGAGTGT | |
| | GGTGA | |
| 11 | ATGGGATGGTCATGTATCATCCTTTTTCTAGTAGCAACTGCA | IgG-CTF-2 format, |
| | ACCGGTGTACATTCAGACATCCAGATGACCCAGTCTCCATCC | neprolysin, anti-gD, light |
| | TCCCTGTCTGCATCTGTAGGAGACAGAGTCACCATCACTTGC | chain: |
| | CGGGCAAGTGCGTCTGTTGACTCTTACGGTAACAGCTTTATA | |
| | CATTGGTATCAGCAGAAACCAGGGAAAGCCCCTAAGCTCCT | |
| | GATCTATCGTGCATCCGATTTGGAAAGTGGGGTCCCATCAAG | |
| | GTTCAGTGGCAGTGGATCTGGGACAGATTTCACTCTCACCAT | |
| | CAGCAGTCTGCAACCTGAAGATTTTGCAACTTACTACTGTCA | |
| | ACAGAATTACGCTGACCCTTTCACGTTCGGCCAAGGTACCAA | |
| | GGTGGAGATCAAACGAACTGTGGCTGCACCATCTGTCTTCAT | |
| | CTTCCCGCCATCTGATGAGCAGTTGAAATCTGGAACTGCTTC | |
| | TGTTGTGTGCCTGCTGAATAACTTCTATCCCAGAGAGGCCAA | |
| | AGTACAGTGGAAGGTGGATAACGCCCTCCAATCGGGTAACT | |
| | CCCAGGAGAGTGTCACAGAGCAGGACAGCAAGGACAGCACC | |
| | TACAGCCTCAGCAGCACCCTGACGCTGAGCAAAGCAGACTA | |
| | CGAGAAACACAAAGTCTACGCCTGCGAAGTCACCCATCAGG | |
| | GCCTGAGCTCGCCCGTCACAAAGAGCTTCAACAGGGGAGAG | |
| | TGTTAA | |
| 12 | ATGGGATGGTCATGTATCATCCTTTTTCTAGTAGCAACTGCA | IgG-CTF-2 format, |
| | ACCGGTGTACATTCAGAAGTTCAGCTGGTCGAGTCTGGGGGA | neprolysin, crenezumab, |
| | GGCTTAGTGCAGCCTGGAGGGTCCCTGAGACTCTCCTGTGCA | heavy chain: |
| | GCCTCTGGATTCACTTTCAGTAGCTATGGCATGTCTTGGGTTC | |
| | GCCAGGCTCCAGGCAAGGGTCTCGAATTGGTCGCAAGCATC | |
| | AATAGTAATGGTGGTAGCACCTATTATCCAGACAGTGTGAAG | |
| | GGCCGATTCACCATCTCCAGAGACAATGCCAAGAACTCCCTG | |
| | TACCTGCAAATGAACAGTCTGAGAGCTGAGGACACCGCCGT | |
| | GTATTACTGTGCAAGTGGTGACTACTGGGGCCAAGGCACCAC | |
| | TGTCACAGTCTCGAGTGCCTCCACCAAGGGCCCATCGGTCTT | |
| | CCCCCTGGCACCCTCCTCCAAGAGCACCTCTGGGGGCACAGC | |
| | GGCCCTGGGCTGCCTGGTCAAGGACTACTTCCCCGAGCCGGT | |
| | GACGGTGTCGTGGAACTCAGGCGCCCTGACCAGCGGCGTGC | |
| | ACACCTTCCCGGCTGTCCTACAGTCCTCAGGACTCTACTCCCT | |
| | CAGCAGCGTGGTGACTGTGCCCTCTAGCAGCTTGGGCACCCA | |
| | GACCTACATCTGCAACGTGAATCACAAGCCCAGCAACACCA | |
| | AGGTGGACAAGAAAGTTGAGCCCAAATCTTGTGACAAAACT | |
| | CACACATGCCCACCGTGCCCAGCACCTGAACTCCTGGGGGGA | |
| | CCGTCAGTCTTCCTCTTCCCCCCAAAACCCAAGGACACCCTC | |
| | ATGATCTCCCGGACCCCTGAGGTCACATGCGTGGTGGTGGAC | |
| | GTGAGCCACGAAGACCCTGAGGTCAAGTTCAACTGGTACGT | |
| | GGACGGCGTGGAGGTGCATAATGCCAAGACAAAGCCGCGGG | |
| | AGGAGCAGTACAACAGCACGTACCGTGTGGTCAGCGTCCTC | |
| | ACCGTCCTGCACCAGGACTGGCTGAATGGCAAGGAGTACAA | |
| | GTGCAAGGTCTCCAACAAAGCCCTCCCAGCCCCCATCGAGAA | |
| | AACCATCTCCAAAGCCAAAGGGCAGCCCCGAGAACCACAGG | |
| | TGTACACCCTGCCCCCATCCCGGGAAGAGATGACCAAGAAC | |
| | CAGGTCAGCCTGACCTGCCTGGTCAAAGGCTTCTATCCCAGC | |
| | GACATCGCCGTGGAGTGGGAGAGCAATGGGCAGCCGGAGAA | |
| | CAACTACAAGACCACGCCTCCCGTGCTGGACTCCGACGGCTC | |
| | CTTCTTCCTCTACAGCAAGCTCACCGTGGACAAGAGCAGGTG | |
| | GCAGCAGGGGAACGTCTTCTCATGCTCCGTGATGCATGAGGC | |
| | TCTGCACAACCACTACACGCAGAAGAGCCTCTCCCTGTCTCC | |
| | GGGTGGCGGCGGCGGATCCTACGACGACGGCATCTGCAAGA | |
| | GCAGCGACTGCATCAAGAGCGCCGCCAGACTGATCCAGAAC | |
| | ATGGACGCCACCACCGAGCCCTGCACCGACTTCTTCAAGTAC | |
| | GCCTGCGGCGGCTGGCTGAAGAGAAACGTGATCCCCGAGAC | |
| | CAGCAGCAGATACGGCAACTTCGACATCCTGAGAGACGAGC | |
| | TGGAGGTGGTGCTGAAGGATGTGCTGCAGGAGCCCAAGACC | |
| | GAGGACATCGTGGCCGTGCAGAAGGCCAAGGCCCTGTACAG | |
| | AAGCTGCATCAACGAGAGCGCCATCGACAGCAGAGGCGGCG | |
| | AGCCCCTGCTGAAGCTGCTGCCCGACATCTACGGCTGGCCCG | |
| | TGGCCACCGAGAACTGGGAGCAGAAGTACGGCGCCAGCTGG | |
| | ACCGCCGAGAAGGCCATCGCCCAGCTGAACAGCAAGTACGG | |
| | CAAGAAGGTGCTGATCAACCTGTTCGTGGGCACCGACGACA | |
| | AGAACAGCGTGAACCACGTGATCCACATCGACCAGCCCAGA | |
| | CTGGGCCTGCCCAGCAGAGACTACTACGAGTGCACCGGCATC | |
| | TACAAGGAGGCCTGCACCGCCTACGTGGACTTCATGATCAGC | |
| | GTGGCCAGACTGATCAGACAGGAGGAGAGACTGCCCATCGA | |
| | CGAGAACCAGCTGGCCCTGGAGATGAACAAGGTGATGGAGC | |
| | TGGAGAAGGAGATCGCCAACGCCACCGCCAAGCCCGAGGAC | |
| | AGAAACGACCCCATGCTGCTGTACAACAAGATGACCCTGGC | |
| | CCAGATCCAGAACAACTTCAGCCTGGAGATCAACGGCAAGC | |
| | CCTTCAGCTGGCTGAACTTCACCAACGAGATCATGAGCACCG | |
| | TGAACATCAGCATCACCAACGAGGAGGATGTGGTGGTGTAC | |
| | GCCCCCGAGTACCTGACCAAGCTGAAGCCCATCCTGACCAAG | |
| | TACAGCGCCAGAGACCTGCAGAACCTGATGAGCTGGAGATT | |
| | CATCATGGACCTGGTGAGCAGCCTGAGCAGAACCTACAAGG | |
| | AGAGCAGAAACGCCTTCAGAAAGGCCCTGTACGGCACCACC | |
| | AGCGAGACCGCCACCTGGAGAAGATGCGCCAACTACGTGAA | |
| | CGGCAACATGGAGAACGCCGTGGGCAGACTGTACGTGGAGG | |
| | CCGCCTTCGCCGGCGAGAGCAAGCACGTGGTGGAGGACCTG | |
| | ATCGCCCAGATCAGAGAGGTGTTCATCCAGACCCTGGACGAC | |
| | CTGACCTGGATGGACGCCGAGACCAAGAAGAGAGCCGAGGA | |
| | GAAGGCCCTGGCCATCAAGGAGAGAATCGGCTACCCCGACG | |
| | ACATCGTGAGCAACGACAACAAGCTGAACAACGAGTACCTG | |
| | GAGCTGAACTACAAGGAGGACGAGTACTTCGAGAACATCAT | |
| | CCAGAACCTGAAGTTCAGCCAGAGCAAGCAGCTGAAGAAGC | |
| | TGAGAGAGAAGGTGGACAAGGACGAGTGGATCAGCGGCGCC | |
| | GCCGTGGTGAACGCCTTCTACAGCAGCGGCAGAAACCAGAT | |
| | CGTGTTCCCCGCCGGCATCCTGCAGCCCCCCTTCTTCAGCGCC | |
| | CAGCAGAGCAACAGCCTGAACTACGGCGGCATCGGCATGGT | |
| | GATCGGCCACGAGATCACCCACGGCTTCGACGACAACGGCA | |
| | GAAACTTCAACAAGGACGGCGACCTGGTGGACTGGTGGACC | |
| | CAGCAGAGCGCCAGCAACTTCAAGGAGCAGAGCCAGTGCAT | |
| | GGTGTACCAGTACGGCAACTTCAGCTGGGACCTGGCCGGCG | |
| | GCCAGCACCTGAACGGCATCAACACCCTGGGCGAGAACATC | |
| | GCCGACAACGGCGGCCTGGGCCAGGCCTACAGAGCCTACCA | |
| | GAACTACATCAAGAAGAACGGCGAGGAGAAGCTGCTGCCCG | |
| | GCCTGGACCTGAACCACAAGCAGCTGTTCTTCCTGAACTTCG | |
| | CCCAGGTGTGGTGCGGCACCTACAGACCCGAGTACGCCGTG | |
| | AACAGCATCAAGACCGATGTGCACAGCCCCGGCAACTTCAG | |
| | AATCATCGGCACCCTGCAGAACAGCGCCGAGTTCAGCGAGG | |
| | CCTTCCACTGCAGAAAGAACAGCTACATGAACCCCGAGAAG | |
| | AAGTGCAGAGTGTGGTGA | |
| 13 | ATGGGATGGTCATGTATCATCCTTTTTCTAGTAGCAACTGCA | IgG-CTF-2 format, |
| | ACCGGTGTACATTCAGATATCGTGATGACCCAATCTCCACTC | neprolysin, crenezumab, light |
| | TCCCTGCCTGTCACTCCTGGTGAGCCTGCCTCCATCTCTTGCA | chain: |
| | GATCTAGTCAGAGCCTTGTATATAGTAATGGAGACACCTATT | |
| | TACATTGGTATCTGCAGAAGCCAGGCCAGTCTCCACAGCTCC | |
| | TGATCTACAAAGTTTCCAACCGATTTTCTGGGGTCCCAGACA | |
| | GGTTCAGTGGCAGTGGATCAGGGACAGATTTCACACTCAAG | |
| | ATCAGCAGAGTGGAGGCTGAGGATGTGGGAGTTTATTACTGC | |
| | TCTCAAAGTACACATGTTCCTTGGACGTTCGGCCAAGGTACC | |
| | AAGGTGGAGATCAAACGAACTGTGGCTGCACCATCTGTCTTC | |
| | ATCTTCCCGCCATCTGATGAGCAGTTGAAATCTGGAACTGCT | |
| | TCTGTTGTGTGCCTGCTGAATAACTTCTATCCCAGAGAGGCC | |
| | AAAGTACAGTGGAAGGTGGATAACGCCCTCCAATCGGGTAA | |
| | CTCCCAGGAGAGTGTCACAGAGCAGGACAGCAAGGACAGCA | |
| | CCTACAGCCTCAGCAGCACCCTGACGCTGAGCAAAGCAGAC | |
| | TACGAGAAACACAAAGTCTACGCCTGCGAAGTCACCCATCA | |
| | GGGCCTGAGCTCGCCCGTCACAAAGAGCTTCAACAGGGGAG | |
| | AGTGTTAA | |
| 14 | ATGGGATGGTCATGTATCATCCTTTTTCTAGTAGCAACTGCA | IgG-CTF-2 format, |
| | ACCGGTGTACATTCAGAAGTGCAGCTGGTGGAGTCTGGGGG | neprolysin, solanezumab, |
| | AGGCTTGGTACAGCCTGGGGGGTCCCTGAGACTCTCCTGTGC | heavy chain: |
| | AGCCTCTGGATTCACCTTTAGCAGATATTCCATGAGCTGGGT | |
| | CCGCCAGGCTCCAGGGAAGGGGCTGGAGCTGGTCGCACAAA | |
| | TTAATAGCGTTGGTAATAGCACATACTATCCAGACACCGTGA | |
| | AGGGCCGGTTCACCATCTCCAGAGATAATGCCAAGAACACG | |
| | CTGTATCTGCAAATGAACAGCCTGAGAGCCGAGGACACGGC | |
| | CGTATATTACTGTGCGTCAGGTGACTACTGGGGCCAAGGAAC | |
| | CCTGGTCACCGTCTCGAGTGCCTCCACCAAGGGCCCATCGGT | |
| | CTTCCCCCTGGCACCCTCCTCCAAGAGCACCTCTGGGGGCAC | |
| | AGCGGCCCTGGGCTGCCTGGTCAAGGACTACTTCCCCGAGCC | |
| | GGTGACGGTGTCGTGGAACTCAGGCGCCCTGACCAGCGGCG | |
| | TGCACACCTTCCCGGCTGTCCTACAGTCCTCAGGACTCTACTC | |
| | CCTCAGCAGCGTGGTGACTGTGCCCTCTAGCAGCTTGGGCAC | |
| | CCAGACCTACATCTGCAACGTGAATCACAAGCCCAGCAACA | |
| | CCAAGGTGGACAAGAAAGTTGAGCCCAAATCTTGTGACAAA | |
| | ACTCACACATGCCCACCGTGCCCAGCACCTGAACTCCTGGGG | |
| | GGACCGTCAGTCTTCCTCTTCCCCCCAAAACCCAAGGACACC | |
| | CTCATGATCTCCCGGACCCCTGAGGTCACATGCGTGGTGGTG | |
| | GACGTGAGCCACGAAGACCCTGAGGTCAAGTTCAACTGGTA | |
| | CGTGGACGGCGTGGAGGTGCATAATGCCAAGACAAAGCCGC | |
| | GGGAGGAGCAGTACAACAGCACGTACCGTGTGGTCAGCGTC | |
| | CTCACCGTCCTGCACCAGGACTGGCTGAATGGCAAGGAGTAC | |
| | AAGTGCAAGGTCTCCAACAAAGCCCTCCCAGCCCCCATCGAG | |
| | AAAACCATCTCCAAAGCCAAAGGGCAGCCCCGAGAACCACA | |
| | GGTGTACACCCTGCCCCCATCCCGGGAAGAGATGACCAAGA | |
| | ACCAGGTCAGCCTGACCTGCCTGGTCAAAGGCTTCTATCCCA | |
| | GCGACATCGCCGTGGAGTGGGAGAGCAATGGGCAGCCGGAG | |
| | AACAACTACAAGACCACGCCTCCCGTGCTGGACTCCGACGGC | |
| | TCCTTCTTCCTCTACAGCAAGCTCACCGTGGACAAGAGCAGG | |
| | TGGCAGCAGGGGAACGTCTTCTCATGCTCCGTGATGCATGAG | |
| | GCTCTGCACAACCACTACACGCAGAAGAGCCTCTCCCTGTCT | |
| | CCGGGTGGCGGCGGCGGATCCTACGACGACGGCATCTGCAA | |
| | GAGCAGCGACTGCATCAAGAGCGCCGCCAGACTGATCCAGA | |
| | ACATGGACGCCACCACCGAGCCCTGCACCGACTTCTTCAAGT | |
| | ACGCCTGCGGCGGCTGGCTGAAGAGAAACGTGATCCCCGAG | |
| | ACCAGCAGCAGATACGGCAACTTCGACATCCTGAGAGACGA | |
| | GCTGGAGGTGGTGCTGAAGGATGTGCTGCAGGAGCCCAAGA | |
| | CCGAGGACATCGTGGCCGTGCAGAAGGCCAAGGCCCTGTAC | |
| | AGAAGCTGCATCAACGAGAGCGCCATCGACAGCAGAGGCGG | |
| | CGAGCCCCTGCTGAAGCTGCTGCCCGACATCTACGGCTGGCC | |
| | CGTGGCCACCGAGAACTGGGAGCAGAAGTACGGCGCCAGCT | |
| | GGACCGCCGAGAAGGCCATCGCCCAGCTGAACAGCAAGTAC | |
| | GGCAAGAAGGTGCTGATCAACCTGTTCGTGGGCACCGACGA | |
| | CAAGAACAGCGTGAACCACGTGATCCACATCGACCAGCCCA | |
| | GACTGGGCCTGCCCAGCAGAGACTACTACGAGTGCACCGGC | |
| | ATCTACAAGGAGGCCTGCACCGCCTACGTGGACTTCATGATC | |
| | AGCGTGGCCAGACTGATCAGACAGGAGGAGAGACTGCCCAT | |
| | CGACGAGAACCAGCTGGCCCTGGAGATGAACAAGGTGATGG | |
| | AGCTGGAGAAGGAGATCGCCAACGCCACCGCCAAGCCCGAG | |
| | GACAGAAACGACCCCATGCTGCTGTACAACAAGATGACCCT | |
| | GGCCCAGATCCAGAACAACTTCAGCCTGGAGATCAACGGCA | |
| | AGCCCTTCAGCTGGCTGAACTTCACCAACGAGATCATGAGCA | |
| | CCGTGAACATCAGCATCACCAACGAGGAGGATGTGGTGGTG | |
| | TACGCCCCCGAGTACCTGACCAAGCTGAAGCCCATCCTGACC | |
| | AAGTACAGCGCCAGAGACCTGCAGAACCTGATGAGCTGGAG | |
| | ATTCATCATGGACCTGGTGAGCAGCCTGAGCAGAACCTACAA | |
| | GGAGAGCAGAAACGCCTTCAGAAAGGCCCTGTACGGCACCA | |
| | CCAGCGAGACCGCCACCTGGAGAAGATGCGCCAACTACGTG | |
| | AACGGCAACATGGAGAACGCCGTGGGCAGACTGTACGTGGA | |
| | GGCCGCCTTCGCCGGCGAGAGCAAGCACGTGGTGGAGGACC | |
| | TGATCGCCCAGATCAGAGAGGTGTTCATCCAGACCCTGGACG | |
| | ACCTGACCTGGATGGACGCCGAGACCAAGAAGAGAGCCGAG | |
| | GAGAAGGCCCTGGCCATCAAGGAGAGAATCGGCTACCCCGA | |
| | CGACATCGTGAGCAACGACAACAAGCTGAACAACGAGTACC | |
| | TGGAGCTGAACTACAAGGAGGACGAGTACTTCGAGAACATC | |
| | ATCCAGAACCTGAAGTTCAGCCAGAGCAAGCAGCTGAAGAA | |
| | GCTGAGAGAGAAGGTGGACAAGGACGAGTGGATCAGCGGCG | |
| | CCGCCGTGGTGAACGCCTTCTACAGCAGCGGCAGAAACCAG | |
| | ATCGTGTTCCCCGCCGGCATCCTGCAGCCCCCCTTCTTCAGCG | |
| | CCCAGCAGAGCAACAGCCTGAACTACGGCGGCATCGGCATG | |
| | GTGATCGGCCACGAGATCACCCACGGCTTCGACGACAACGG | |
| | CAGAAACTTCAACAAGGACGGCGACCTGGTGGACTGGTGGA | |
| | CCCAGCAGAGCGCCAGCAACTTCAAGGAGCAGAGCCAGTGC | |
| | ATGGTGTACCAGTACGGCAACTTCAGCTGGGACCTGGCCGGC | |
| | GGCCAGCACCTGAACGGCATCAACACCCTGGGCGAGAACAT | |
| | CGCCGACAACGGCGGCCTGGGCCAGGCCTACAGAGCCTACC | |
| | AGAACTACATCAAGAAGAACGGCGAGGAGAAGCTGCTGCCC | |
| | GGCCTGGACCTGAACCACAAGCAGCTGTTCTTCCTGAACTTC | |
| | GCCCAGGTGTGGTGCGGCACCTACAGACCCGAGTACGCCGT | |
| | GAACAGCATCAAGACCGATGTGCACAGCCCCGGCAACTTCA | |
| | GAATCATCGGCACCCTGCAGAACAGCGCCGAGTTCAGCGAG | |
| | GCCTTCCACTGCAGAAAGAACAGCTACATGAACCCCGAGAA | |
| | GAAGTGCAGAGTGTGGTGA | |
| 15 | ATGGGATGGTCATGTATCATCCTTTTTCTAGTAGCAACTGCA | IgG-CTF-2 format, |
| | ACCGGTGTACATTCAGATGTTGTGATGACTCAATCTCCACTC | neprolysin, solanezumab, |
| | TCCCTGCCCGTCACCCTTGGACAGCCAGCCTCCATCTCATGC | light chain: |
| | AGGAGTAGTCAAAGCCTCATATACAGTGATGGAAACGCCTA | |
| | CTTGCATTGGTTTCTCCAGAAGCCAGGCCAATCTCCAAGGCT | |
| | CCTAATTTATAAGGTTTCTAACAGATTCTCTGGCGTCCCAGA | |
| | CAGATTCAGCGGCAGTGGGTCAGGCACTGATTTCACACTGAA | |
| | AATCAGCAGGGTGGAGGCTGAGGATGTTGGCGTGTATTACTG | |
| | CTCGCAATCTACTCACGTTCCTTGGACGTTCGGCCAAGGTAC | |
| | CAAGGTGGAGATCAAACGAACTGTGGCTGCACCATCTGTCTT | |
| | CATCTTCCCGCCATCTGATGAGCAGTTGAAATCTGGAACTGC | |
| | TTCTGTTGTGTGCCTGCTGAATAACTTCTATCCCAGAGAGGC | |
| | CAAAGTACAGTGGAAGGTGGATAACGCCCTCCAATCGGGTA | |
| | ACTCCCAGGAGAGTGTCACAGAGCAGGACAGCAAGGACAGC | |
| | ACCTACAGCCTCAGCAGCACCCTGACGCTGAGCAAAGCAGA | |
| | CTACGAGAAACACAAAGTCTACGCCTGCGAAGTCACCCATC | |
| | AGGGCCTGAGCTCGCCCGTCACAAAGAGCTTCAACAGGGGA | |
| | GAGTGTTAA | |
| 16 | ATGGGATGGTCATGTATCATCCTTTTTCTAGTAGCAACTGCA | IgG-NTF(LC)-2 format, |
| | ACCGGTGTACATTCAGAGGTTCAGCTGGTGGAGTCTGGCGGT | MMP3-D4K-4, anti-gD, |
| | GGCCTGGTGCAGCCAGGGGGCTCACTCCGTTTGTCCTGTGCC | heavy chain: |
| | GCTTCTGGCTACTCCATCACCTCCGACTTTGCCTGGAACTGG | |
| | GTCCGTCAGGCCCCGGGTAAGGGCCTGGAATGGGTTGGATA | |
| | CATTAGTTACTCTGGAACCACTAGCTATAACCCTAGCCTGAA | |
| | GTCCCGTATCACTATAAGTCGCGACAATTCCAAAAACACATT | |
| | CTACCTGCAGATGAACAGCCTGCGTGCTGAGGACACTGCCGT | |
| | CTATTATTGTGCTCGGGAAAACTACTATGGCCGTTCTCACGTT | |
| | GGGTACTTCGACGTCTGGGGTCAAGGAACCCTGGTCACCGTC | |
| | TCGAGTGCCTCCACCAAGGGCCCATCGGTCTTCCCCCTGGCA | |
| | CCCTCCTCCAAGAGCACCTCTGGGGGCACAGCGGCCCTGGGC | |
| | TGCCTGGTCAAGGACTACTTCCCCGAGCCGGTGACGGTGTCG | |
| | TGGAACTCAGGCGCCCTGACCAGCGGCGTGCACACCTTCCCG | |
| | GCTGTCCTACAGTCCTCAGGACTCTACTCCCTCAGCAGCGTG | |
| | GTGACTGTGCCCTCTAGCAGCTTGGGCACCCAGACCTACATC | |
| | TGCAACGTGAATCACAAGCCCAGCAACACCAAGGTGGACAA | |
| | GAAAGTTGAGCCCAAATCTTGTGACAAAACTCACACATGCCC | |
| | ACCGTGCGGCGGAGGCGGAGCCGGCGGGGGACCGTCAGTCT | |
| | TCCTCTTCCCCCCAAAACCCAAGGACACCCTCATGATCTCCC | |
| | GGACCCCTGAGGTCACATGCGTGGTGGTGGACGTGAGCCAC | |
| | GAAGACCCTGAGGTCAAGTTCAACTGGTACGTGGACGGCGT | |
| | GGAGGTGCATAATGCCAAGACAAAGCCGCGGGAGGAGCAGT | |
| | ACAACAGCACGTACCGTGTGGTCAGCGTCCTCACCGTCCTGC | |
| | ACCAGGACTGGCTGAATGGCAAGGAGTACAAGTGCAAGGTC | |
| | TCCAACAAAGCCCTCGGAGCCCCCATCGAGAAAACCATCTCC | |
| | AAAGCCAAAGGGCAGCCCCGAGAACCACAGGTGTACACCCT | |
| | GCCCCCATCCGAGGAAGAGATGACCAAGAACCAGGTCAGCC | |
| | TGACCTGCCTGGTCAAAGGCTTCTATCCCAGCGACATCGCCG | |
| | TGGAGTGGGAGAGCAATGGGCAGCCGGAGAACAACTACAAG | |
| | ACCACGCCTCCCGTGCTGGACTCCGACGGCTCCTTCTTCCTCT | |
| | ACAGCAAGCTCACCGTGGACAAGAGCAGGTGGCAGCAGGGG | |
| | AACGTCTTCTCATGCTCCGTGATGCATGAGGCTCTGCACAAC | |
| | CACTACACGCAGAAGAGCCTCTCCCTGTCTCCGGGTTGA | |
| 17 | ATGGGATGGTCATGTATCATCCTTTTTCTAGTAGCAACTGCA | IgG-NTF(LC)-2 format, |
| | ACCGGTGTACATTCATACCCCCTGGACGGCGCCGCTAGAGGC | MMP3-D4K-4, anti-gD, light |
| | GAGGACACCAGCATGAACCTGGTGCAGAAGTACCTGGAGAA | chain: |
| | CTACTATGACCTGAAGAAGGACGATGACGATAAGGTGAGAA | |
| | GGAAGGACAGCGGCCCCGTGGTCAAGAAGATCAGAGAGATG | |
| | CAGAAGTTCCTGGGCCTGGAGGTGACCGGCAAGCTGGACAG | |
| | CGACACCCTGGAGGTGATGAGAAAGCCCAGATGCGGCGTGC | |
| | CCGACGTGGGCCACTTCAGAACCTTCCCCGGCATCCCCAAGT | |
| | GGAGAAAGACCCACCTGACCTACAGAATCGTGAACTACACC | |
| | CCCGACCTGCCCAAGGACGCCGTGGACAGCGCCGTGGAGAA | |
| | GGCCCTGAAGGTGTGGGAGGAAGTGACCCCCCTGACCTTCA | |
| | GCAGACTGTACGAGGGCGAGGCCGACATCATGATCAGCTTC | |
| | GCCGTGAGAGAGCACGGCGACTTCTACCCCTTCGACGGCCCC | |
| | GGCAACGTGCTGGCCCACGCCTACGCCCCCGGCCCCGGCATC | |
| | AACGGCGACGCCCACTTCGACGATGACGAGCAGTGGACCAA | |
| | GGACACCACAGGCACCAACCTGTTCCTGGTGGCCGCTCACGA | |
| | GATCGGCCACAGCCTGGGCCTGTTCCACAGCGCCAACACCGA | |
| | GGCCCTGATGTACCCCCTGTACCACAGCCTGACCGACCTGAC | |
| | CAGATTCAGACTGAGCCAGGACGATATCAACGGCATCCAGA | |
| | GCCTGTACGGCCCTCCACCTGACAGCCCCGAGACCCCCCTGG | |
| | TGCCCACCGAGCCCGTGCCTCCAGAGCCCGGCACCCCCGCCA | |
| | ACTGCGACCCCGCCCTGAGCTTCGACGCCGTGAGCACCCTGA | |
| | GAGGCGAGATCCTGATCTTCAAGGACAGACACTTCTGGAGA | |
| | AAGAGCCTGAGAAAGCTGGAGCCCGAGCTGCACCTGATCAG | |
| | CTCCTTCTGGCCCAGCCTGCCCAGCGGCGTGGACGCCGCTTA | |
| | CGAGGTGACCAGCAAGGACCTGGTGTTCATCTTCAAGGGCA | |
| | ACCAGTTCTGGGCCATCAGAGGCAACGAGGTGAGAGCCGGC | |
| | TACCCCAGAGGCATCCACACCCTGGGCTTCCCTCCAACCGTG | |
| | AGAAAGATCGACGCCGCTATCAGCGACAAGGAGAAGAACAA | |
| | GACCTACTTCTTCGTGGAGGACAAGTACTGGAGATTCGACGA | |
| | GAAGAGAAACAGCATGGAGCCCGGCTTCCCCAAGCAGATCG | |
| | CCGAGGACTTCCCCGGCATCGACAGCAAGATCGACGCCGTGT | |
| | TCGAGGAGTTCGGCTTCTTCTACTTCTTCACCGGCAGCTCCCA | |
| | GCTGGAGTTCGACCCCAACGCCAAGAAGGTGACCCACACCC | |
| | TGAAGAGCAACAGCTGGCTGAACTGCGGCGGAGGCGGAGCC | |
| | GGCGGAGGCGGAGCCGACATCCAGATGACCCAGTCTCCATC | |
| | CTCCCTGTCTGCATCTGTAGGAGACAGAGTCACCATCACTTG | |
| | CCGGGCAAGTGCGTCTGTTGACTCTTACGGTAACAGCTTTAT | |
| | ACATTGGTATCAGCAGAAACCAGGGAAAGCCCCTAAGCTCC | |
| | TGATCTATCGTGCATCCGATTTGGAAAGTGGGGTCCCATCAA | |
| | GGTTCAGTGGCAGTGGATCTGGGACAGATTTCACTCTCACCA | |
| | TCAGCAGTCTGCAACCTGAAGATTTTGCAACTTACTACTGTC | |
| | AACAGAATTACGCTGACCCTTTCACGTTCGGCCAAGGTACCA | |
| | AGGTGGAGATCAAACGAACTGTGGCTGCACCATCTGTCTTCA | |
| | TCTTCCCGCCATCTGATGAGCAGTTGAAATCTGGAACTGCTT | |
| | CTGTTGTGTGCCTGCTGAATAACTTCTATCCCAGAGAGGCCA | |
| | AAGTACAGTGGAAGGTGGATAACGCCCTCCAATCGGGTAAC | |
| | TCCCAGGAGAGTGTCACAGAGCAGGACAGCAAGGACAGCAC | |
| | CTACAGCCTCAGCAGCACCCTGACGCTGAGCAAAGCAGACT | |
| | ACGAGAAACACAAAGTCTACGCCTGCGAAGTCACCCATCAG | |
| | GGCCTGAGCTCGCCCGTCACAAAGAGCTTCAACAGGGGAGA | |
| | GTGTTAA | |
| 18 | ATGGGATGGTCATGTATCATCCTTTTTCTAGTAGCAACTGCA | IgG-NTF(LC)-2 format, |
| | ACCGGTGTACATTCACAGCTGCAGCTGCAGGAGAGCGGCCC | MMP3-D4K-4, RF61 wild- |
| | CGGCCTGGTGAAGCCCAGCGAGACCCTGAGCCTGACCTGCA | type, heavy chain: |
| | CCGTGAGCGGCGGCAGCATCAGCAGAGGCAGCCACTACTGG | |
| | GGCTGGATCAGACAGCCCCCCGGCAAGGGCCTGGAGTGGAT | |
| | CGGCAGCATCTACTACAGCGGCAACACCTACTTCAACCCCAG | |
| | CCTGAAGAGCAGAGTGACCATCAGCGTGGACACCAGCAAGA | |
| | ACCAGTTCAGCCTGAAGCTGAGCAGCGTGACCGCCGCCGAC | |
| | ACCGCCGTGTACTACTGCGCCAGACTGGGCCCCGACGACTAC | |
| | ACCCTGGACGGCATGGACGTGTGGGGCCAGGGCACCACCGT | |
| | GACCGTCTCGAGTGCCTCCACCAAGGGCCCATCGGTCTTCCC | |
| | CCTGGCACCCTCCTCCAAGAGCACCTCTGGGGGCACAGCGGC | |
| | CCTGGGCTGCCTGGTCAAGGACTACTTCCCCGAGCCGGTGAC | |
| | GGTGTCGTGGAACTCAGGCGCCCTGACCAGCGGCGTGCACA | |
| | CCTTCCCGGCTGTCCTACAGTCCTCAGGACTCTACTCCCTCAG | |
| | CAGCGTGGTGACTGTGCCCTCTAGCAGCTTGGGCACCCAGAC | |
| | CTACATCTGCAACGTGAATCACAAGCCCAGCAACACCAAGG | |
| | TGGACAAGAAAGTTGAGCCCAAATCTTGTGACAAAACTCAC | |
| | ACATGCCCACCGTGCGGCGGAGGCGGAGCCGGCGGGGGACC | |
| | GTCAGTCTTCCTCTTCCCCCCAAAACCCAAGGACACCCTCAT | |
| | GATCTCCCGGACCCCTGAGGTCACATGCGTGGTGGTGGACGT | |
| | GAGCCACGAAGACCCTGAGGTCAAGTTCAACTGGTACGTGG | |
| | ACGGCGTGGAGGTGCATAATGCCAAGACAAAGCCGCGGGAG | |
| | GAGCAGTACAACAGCACGTACCGTGTGGTCAGCGTCCTCACC | |
| | GTCCTGCACCAGGACTGGCTGAATGGCAAGGAGTACAAGTG | |
| | CAAGGTCTCCAACAAAGCCCTCGGAGCCCCCATCGAGAAAA | |
| | CCATCTCCAAAGCCAAAGGGCAGCCCCGAGAACCACAGGTG | |
| | TACACCCTGCCCCCATCCGAGGAAGAGATGACCAAGAACCA | |
| | GGTCAGCCTGACCTGCCTGGTCAAAGGCTTCTATCCCAGCGA | |
| | CATCGCCGTGGAGTGGGAGAGCAATGGGCAGCCGGAGAACA | |
| | ACTACAAGACCACGCCTCCCGTGCTGGACTCCGACGGCTCCT | |
| | TCTTCCTCTACAGCAAGCTCACCGTGGACAAGAGCAGGTGGC | |
| | AGCAGGGGAACGTCTTCTCATGCTCCGTGATGCATGAGGCTC | |
| | TGCACAACCACTACACGCAGAAGAGCCTCTCCCTGTCTCCGG | |
| | GTTGA | |
| 19 | ATGGGATGGTCATGTATCATCCTTTTTCTAGTAGCAACTGCA | IgG-NTF(LC)-2 format, |
| | ACCGGTGTACATTCATACCCCCTGGACGGCGCCGCTAGAGGC | MMP3-D4K-4, RF61 wild- |
| | GAGGACACCAGCATGAACCTGGTGCAGAAGTACCTGGAGAA | type, light chain: |
| | CTACTATGACCTGAAGAAGGACGATGACGATAAGGTGAGAA | |
| | GGAAGGACAGCGGCCCCGTGGTCAAGAAGATCAGAGAGATG | |
| | CAGAAGTTCCTGGGCCTGGAGGTGACCGGCAAGCTGGACAG | |
| | CGACACCCTGGAGGTGATGAGAAAGCCCAGATGCGGCGTGC | |
| | CCGACGTGGGCCACTTCAGAACCTTCCCCGGCATCCCCAAGT | |
| | GGAGAAAGACCCACCTGACCTACAGAATCGTGAACTACACC | |
| | CCCGACCTGCCCAAGGACGCCGTGGACAGCGCCGTGGAGAA | |
| | GGCCCTGAAGGTGTGGGAGGAAGTGACCCCCCTGACCTTCA | |
| | GCAGACTGTACGAGGGCGAGGCCGACATCATGATCAGCTTC | |
| | GCCGTGAGAGAGCACGGCGACTTCTACCCCTTCGACGGCCCC | |
| | GGCAACGTGCTGGCCCACGCCTACGCCCCCGGCCCCGGCATC | |
| | AACGGCGACGCCCACTTCGACGATGACGAGCAGTGGACCAA | |
| | GGACACCACAGGCACCAACCTGTTCCTGGTGGCCGCTCACGA | |
| | GATCGGCCACAGCCTGGGCCTGTTCCACAGCGCCAACACCGA | |
| | GGCCCTGATGTACCCCCTGTACCACAGCCTGACCGACCTGAC | |
| | CAGATTCAGACTGAGCCAGGACGATATCAACGGCATCCAGA | |
| | GCCTGTACGGCCCTCCACCTGACAGCCCCGAGACCCCCCTGG | |
| | TGCCCACCGAGCCCGTGCCTCCAGAGCCCGGCACCCCCGCCA | |
| | ACTGCGACCCCGCCCTGAGCTTCGACGCCGTGAGCACCCTGA | |
| | GAGGCGAGATCCTGATCTTCAAGGACAGACACTTCTGGAGA | |
| | AAGAGCCTGAGAAAGCTGGAGCCCGAGCTGCACCTGATCAG | |
| | CTCCTTCTGGCCCAGCCTGCCCAGCGGCGTGGACGCCGCTTA | |
| | CGAGGTGACCAGCAAGGACCTGGTGTTCATCTTCAAGGGCA | |
| | ACCAGTTCTGGGCCATCAGAGGCAACGAGGTGAGAGCCGGC | |
| | TACCCCAGAGGCATCCACACCCTGGGCTTCCCTCCAACCGTG | |
| | AGAAAGATCGACGCCGCTATCAGCGACAAGGAGAAGAACAA | |
| | GACCTACTTCTTCGTGGAGGACAAGTACTGGAGATTCGACGA | |
| | GAAGAGAAACAGCATGGAGCCCGGCTTCCCCAAGCAGATCG | |
| | CCGAGGACTTCCCCGGCATCGACAGCAAGATCGACGCCGTGT | |
| | TCGAGGAGTTCGGCTTCTTCTACTTCTTCACCGGCAGCTCCCA | |
| | GCTGGAGTTCGACCCCAACGCCAAGAAGGTGACCCACACCC | |
| | TGAAGAGCAACAGCTGGCTGAACTGCGGCGGAGGCGGAGCC | |
| | GGCGGAGGCGGAGCCCAGAGCGTGCTGACCCAGCCCCCCAG | |
| | CGCCAGCGGCACCCCCGGCCAGAGAGTGACCATCAGCTGCA | |
| | GCGGCAGCAGCAGCAACATCGGCAGCAACTACGTGTACTGG | |
| | TATCAGCAGCTGCCCGGCACCGCCCCCAAGCTGCTGATCTAC | |
| | AGAAACAACCAGAGACCCAGCGGCGTGCCCGACAGATTCAG | |
| | CGGCAGCAAGAGCGGCACCAGCGCCAGCCTGGCCATCAGCG | |
| | GCCTGAGAAGCGAGGACGAGGCCGACTACTACTGCGCCACC | |
| | TGGGACGACAGCCTGAGCGCCGTGATCTTCGGCGGCGGTACC | |
| | AAGCTGACCGTCCTTGGCCAACCTAAGGCTGCACCATCTGTC | |
| | ACCCTCTTCCCGCCATCTTCTGAGGAGTTGCAAGCTAACAAA | |
| | GCCACTCTTGTGTGCCTGATCAGTGACTTCTATCCCGGAGCG | |
| | GTCACAGTAGCGTGGAAGGCGGATAGCTCCCCCGTAAAGGC | |
| | TGGCGTCGAGACGACTACCCCTTCGAAGCAGAGCAACAACA | |
| | AATACGCCGCCAGCAGCTACCTGTCGCTGACCCCAGAACAGT | |
| | GGAAGAGCCACAAAAGCTACTCCTGCCAAGTCACCCATGAG | |
| | GGCTCGACCGTCGAAAAGACCGTCGCCCCGACAGAGTGTTCT | |
| | TGA | |
| 20 | ATGGGATGGTCATGTATCATCCTTTTTCTAGTAGCAACTGCA | Fab-NTF(LC)-1 format, |
| | ACCGGTGTACATTCACAGCTGCAGCTGCAGGAGAGCGGCCC | MMP3-D4K-4, RF61 wild- |
| | CGGCCTGGTGAAGCCCAGCGAGACCCTGAGCCTGACCTGCA | type, heavy chain: |
| | CCGTGAGCGGCGGCAGCATCAGCAGAGGCAGCCACTACTGG | |
| | GGCTGGATCAGACAGCCCCCCGGCAAGGGCCTGGAGTGGAT | |
| | CGGCAGCATCTACTACAGCGGCAACACCTACTTCAACCCCAG | |
| | CCTGAAGAGCAGAGTGACCATCAGCGTGGACACCAGCAAGA | |
| | ACCAGTTCAGCCTGAAGCTGAGCAGCGTGACCGCCGCCGAC | |
| | ACCGCCGTGTACTACTGCGCCAGACTGGGCCCCGACGACTAC | |
| | ACCCTGGACGGCATGGACGTGTGGGGCCAGGGCACCACCGT | |
| | GACCGTCTCGAGTGCCTCCACCAAGGGCCCATCGGTCTTCCC | |
| | CCTGGCACCCTCCTCCAAGAGCACCTCTGGGGGCACAGCGGC | |
| | CCTGGGCTGCCTGGTCAAGGACTACTTCCCCGAGCCGGTGAC | |
| | GGTGTCGTGGAACTCAGGCGCCCTGACCAGCGGCGTGCACA | |
| | CCTTCCCGGCTGTCCTACAGTCCTCAGGACTCTACTCCCTCAG | |
| | CAGCGTGGTGACTGTGCCCTCTAGCAGCTTGGGCACCCAGAC | |
| | CTACATCTGCAACGTGAATCACAAGCCCAGCAACACCAAGG | |
| | TGGACAAGAAAGTTGAGCCCAAATCTTGTGACAAAACTCAC | |
| | ACAGATTATAAGGACGATGACGATAAATGA | |
| 21 | ATGGGATGGTCATGTATCATCCTTTTTCTAGTAGCAACTGCA | Fab-NTF(LC)-1 format, |
| | ACCGGTGTACATTCATACCCCCTGGACGGCGCCGCTAGAGGC | MMP3-D4K-4, RF61 wild- |
| | GAGGACACCAGCATGAACCTGGTGCAGAAGTACCTGGAGAA | type, light chain: |
| | CTACTATGACCTGAAGAAGGACGATGACGATAAGGTGAGAA | |
| | GGAAGGACAGCGGCCCCGTGGTCAAGAAGATCAGAGAGATG | |
| | CAGAAGTTCCTGGGCCTGGAGGTGACCGGCAAGCTGGACAG | |
| | CGACACCCTGGAGGTGATGAGAAAGCCCAGATGCGGCGTGC | |
| | CCGACGTGGGCCACTTCAGAACCTTCCCCGGCATCCCCAAGT | |
| | GGAGAAAGACCCACCTGACCTACAGAATCGTGAACTACACC | |
| | CCCGACCTGCCCAAGGACGCCGTGGACAGCGCCGTGGAGAA | |
| | GGCCCTGAAGGTGTGGGAGGAAGTGACCCCCCTGACCTTCA | |
| | GCAGACTGTACGAGGGCGAGGCCGACATCATGATCAGCTTC | |
| | GCCGTGAGAGAGCACGGCGACTTCTACCCCTTCGACGGCCCC | |
| | GGCAACGTGCTGGCCCACGCCTACGCCCCCGGCCCCGGCATC | |
| | AACGGCGACGCCCACTTCGACGATGACGAGCAGTGGACCAA | |
| | GGACACCACAGGCACCAACCTGTTCCTGGTGGCCGCTCACGA | |
| | GATCGGCCACAGCCTGGGCCTGTTCCACAGCGCCAACACCGA | |
| | GGCCCTGATGTACCCCCTGTACCACAGCCTGACCGACCTGAC | |
| | CAGATTCAGACTGAGCCAGGACGATATCAACGGCATCCAGA | |
| | GCCTGTACGGCCCTCCACCTGACAGCCCCGAGACCCCCCTGG | |
| | TGCCCACCGAGCCCGTGCCTCCAGAGCCCGGCACCCCCGCCA | |
| | ACTGCGACCCCGCCCTGAGCTTCGACGCCGTGAGCACCCTGA | |
| | GAGGCGAGATCCTGATCTTCAAGGACAGACACTTCTGGAGA | |
| | AAGAGCCTGAGAAAGCTGGAGCCCGAGCTGCACCTGATCAG | |
| | CTCCTTCTGGCCCAGCCTGCCCAGCGGCGTGGACGCCGCTTA | |
| | CGAGGTGACCAGCAAGGACCTGGTGTTCATCTTCAAGGGCA | |
| | ACCAGTTCTGGGCCATCAGAGGCAACGAGGTGAGAGCCGGC | |
| | TACCCCAGAGGCATCCACACCCTGGGCTTCCCTCCAACCGTG | |
| | AGAAAGATCGACGCCGCTATCAGCGACAAGGAGAAGAACAA | |
| | GACCTACTTCTTCGTGGAGGACAAGTACTGGAGATTCGACGA | |
| | GAAGAGAAACAGCATGGAGCCCGGCTTCCCCAAGCAGATCG | |
| | CCGAGGACTTCCCCGGCATCGACAGCAAGATCGACGCCGTGT | |
| | TCGAGGAGTTCGGCTTCTTCTACTTCTTCACCGGCAGCTCCCA | |
| | GCTGGAGTTCGACCCCAACGCCAAGAAGGTGACCCACACCC | |
| | TGAAGAGCAACAGCTGGCTGAACTGCGGCGGAGGCGGAGCC | |
| | GGCGGAGGCGGAGCCCAGAGCGTGCTGACCCAGCCCCCCAG | |
| | CGCCAGCGGCACCCCCGGCCAGAGAGTGACCATCAGCTGCA | |
| | GCGGCAGCAGCAGCAACATCGGCAGCAACTACGTGTACTGG | |
| | TATCAGCAGCTGCCCGGCACCGCCCCCAAGCTGCTGATCTAC | |
| | AGAAACAACCAGAGACCCAGCGGCGTGCCCGACAGATTCAG | |
| | CGGCAGCAAGAGCGGCACCAGCGCCAGCCTGGCCATCAGCG | |
| | GCCTGAGAAGCGAGGACGAGGCCGACTACTACTGCGCCACC | |
| | TGGGACGACAGCCTGAGCGCCGTGATCTTCGGCGGCGGTACC | |
| | AAGCTGACCGTCCTTGGCCAACCTAAGGCTGCACCATCTGTC | |
| | ACCCTCTTCCCGCCATCTTCTGAGGAGTTGCAAGCTAACAAA | |
| | GCCACTCTTGTGTGCCTGATCAGTGACTTCTATCCCGGAGCG | |
| | GTCACAGTAGCGTGGAAGGCGGATAGCTCCCCCGTAAAGGC | |
| | TGGCGTCGAGACGACTACCCCTTCGAAGCAGAGCAACAACA | |
| | AATACGCCGCCAGCAGCTACCTGTCGCTGACCCCAGAACAGT | |
| | GGAAGAGCCACAAAAGCTACTCCTGCCAAGTCACCCATGAG | |
| | GGCTCGACCGTCGAAAAGACCGTCGCCCCGACAGAGTGTTCT | |
| | TGA | |

### EXAMPLES

### Example 1: Expression and Evaluation of Antibody Guided Protease Activity

This Example demonstrates that antibody-enzyme fusions have the specificity and long serum half-life of an antibody while demonstrating the high substrate turnover yet low dose requirements of an enzyme, and that suppression of Aβ was improved through antibody co-targeting of an enzyme to Aβ substrate.

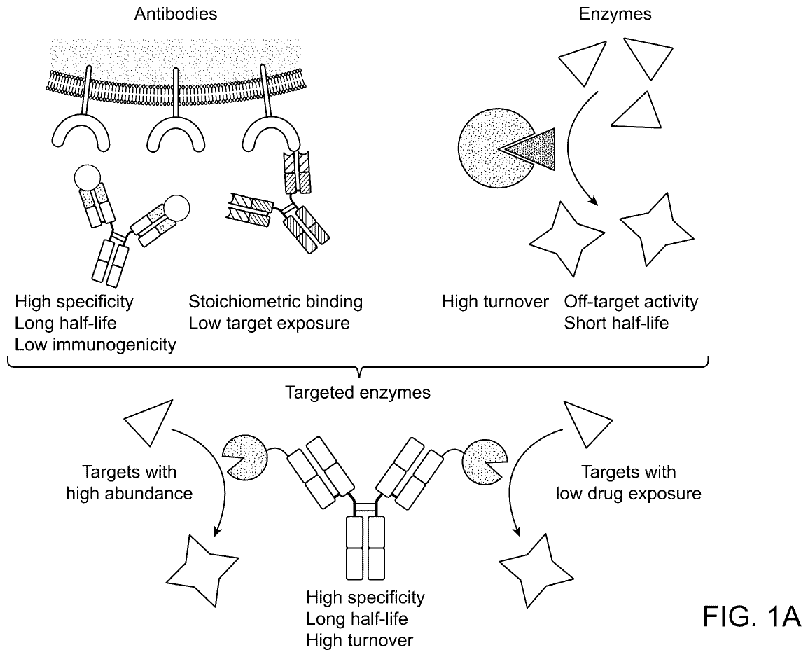

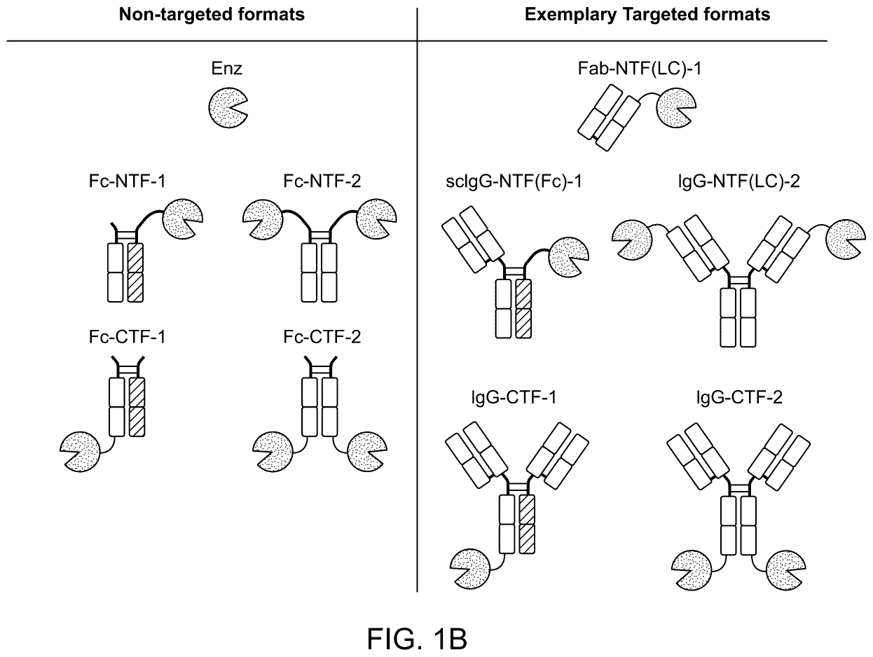

A series of formats of fusions targeting Aβ were engineered that explored geometry and valency, including both N- and C-terminal fusions with either one or two enzymes per molecule (FIGS. 1A-B). N-terminal enzymes were either fused to the antibody light chain (LC) or fragment crystallizable region (Fc), whereas C-terminal fusions were fused to the CH3 domain of the heavy chain (HC). Fc fusion versions lacking targeting arms or IgG formats targeting glycoprotein D of Herpes Simplex Virus (gD) were constructed and tested as non-targeted controls.

Proteolytic degradation of Aβ is an important and natural elimination process to avoid pathogenic accumulation, and as a consequence a diverse set of proteases have been found to play biological roles in endogenous Aβ elimination. To select an optimal protease for our targeted catalyst approach, a set of nine proteases previously implicated in Aβ degradation was screened, including neprolysin (NEP), neprolysin-2 (NEP2), endothelin-converting enzyme 1 and 2 (ECE1 and ECE2), angiotensin-converting enzyme (ACE), insulin-degrading enzyme (IDE), matrix metalloproteinase 2 and 9 (MMP2 and MMP9), and matriptase (MTSP1).

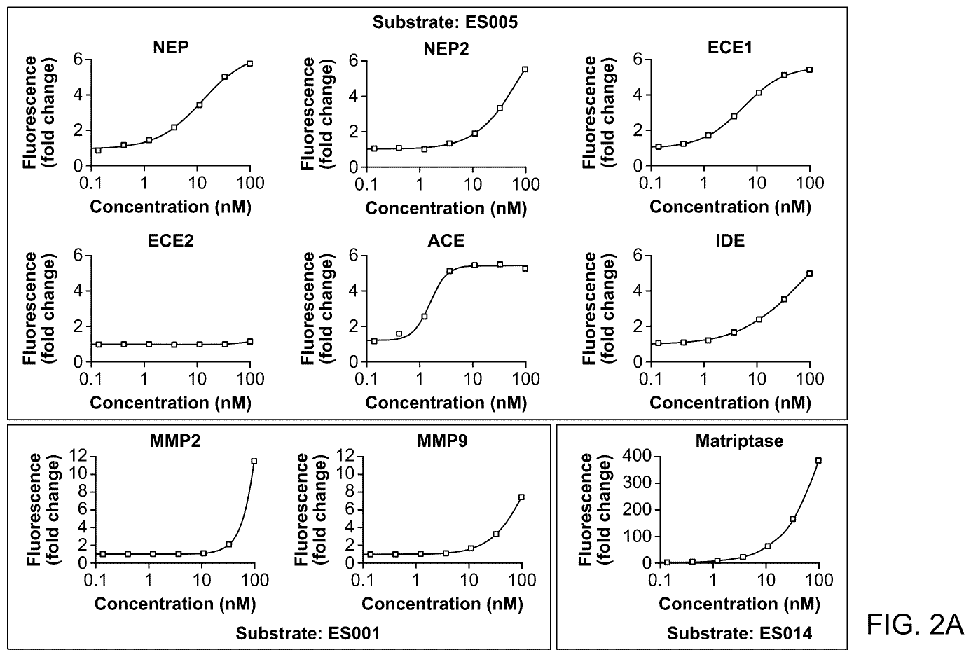

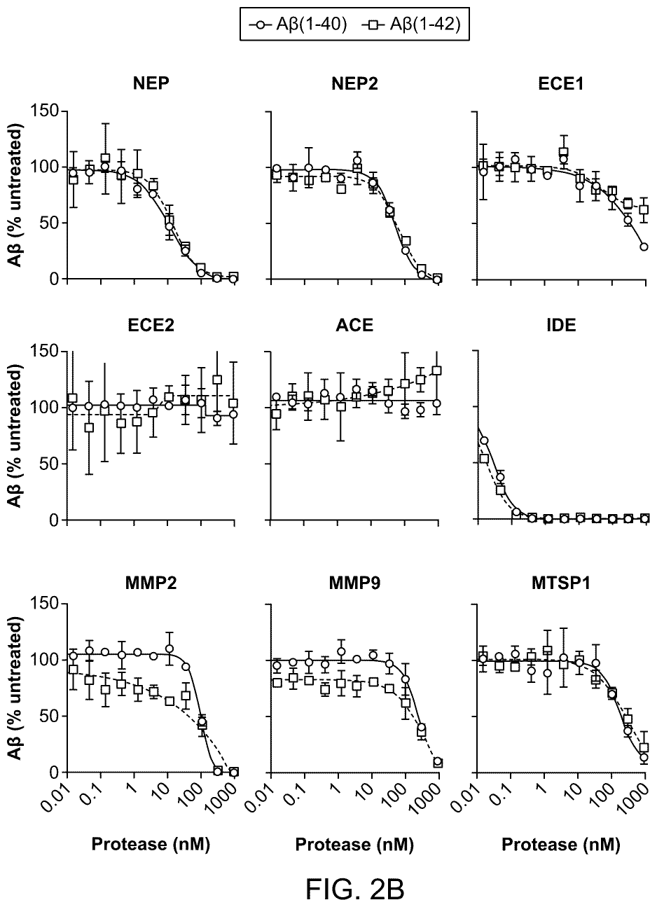

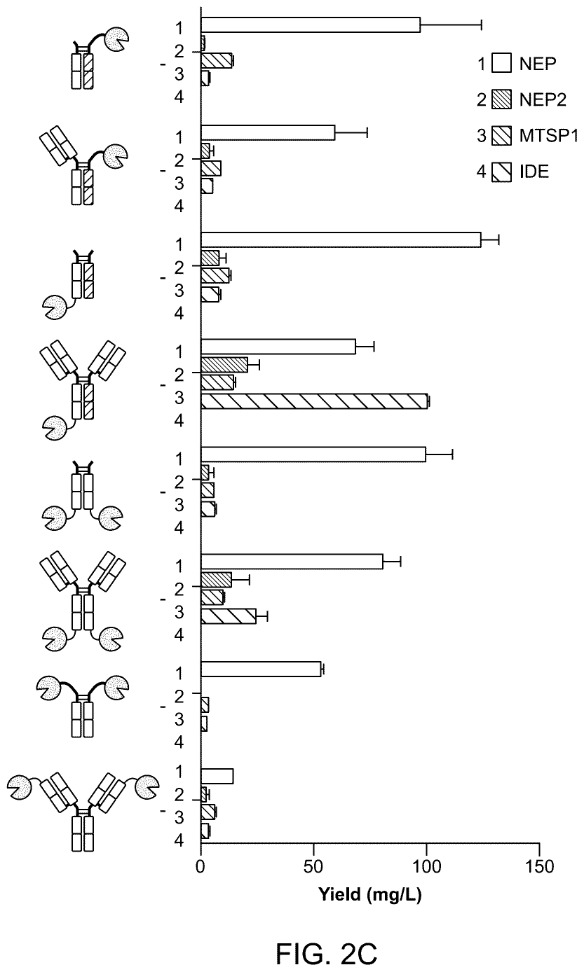

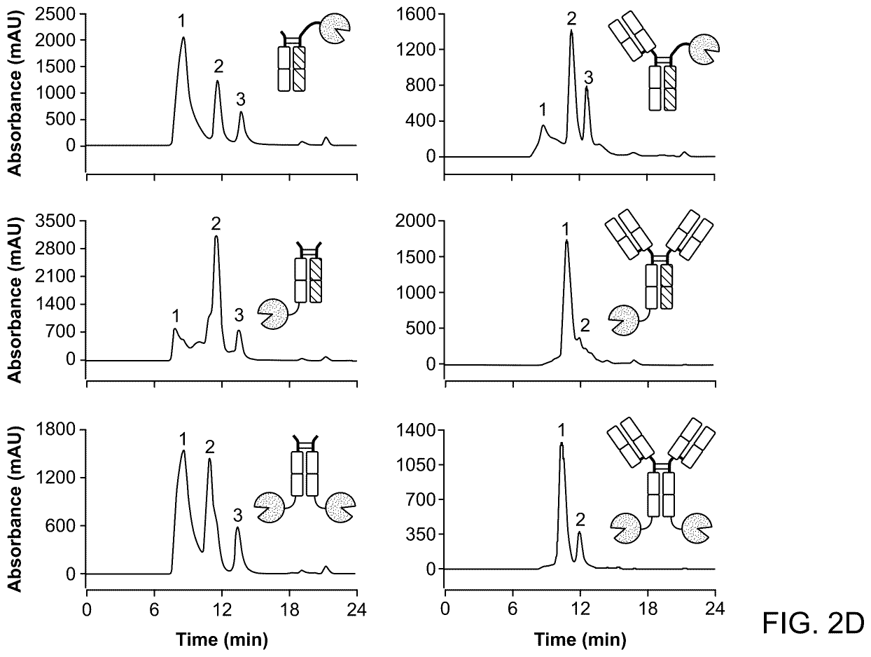

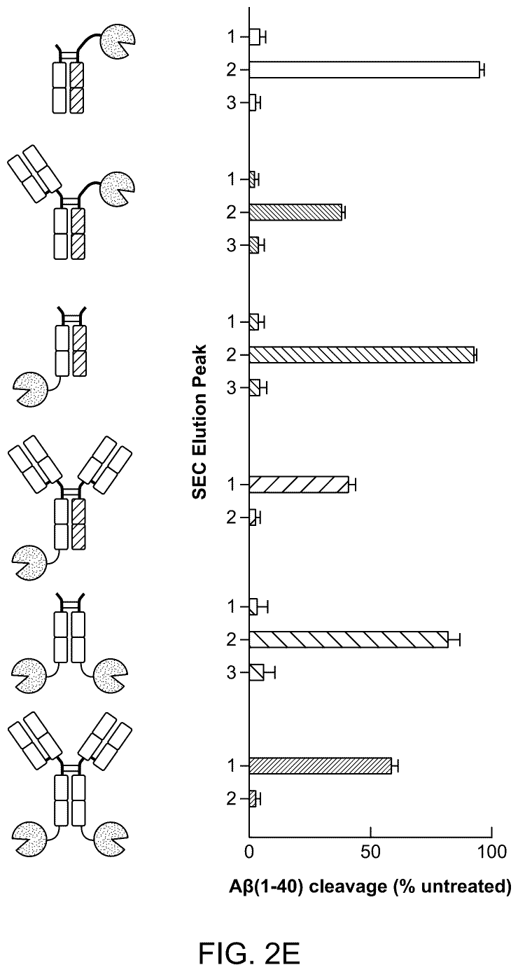

The activity of each protease was first tested on commercially available fluorogenic substrates (FIG. 2A). All enzymes were active on control substrates with the exception of ECE2, which is known to have optimal activity at low pH. The activity of each enzyme was then determined on 2 isoforms of Aβ, 1-40 and 1-42, using an optimized ELISA-based assay that utilizes capture and detection antibodies specific for the N- and C-terminus of the Aβ peptides, respectively. The efficiency of Aβ cleavage was variable with a similar level of activity observed between the two isoforms (FIG. 2B). The most active enzymes against Aβ were IDE (EC50˜0.1 nM) and NEP (EC50˜12 nM), while ECE2 and ACE were completely inactive on both Aβ isoforms.

In order to identify the optimal geometry for the Aβ-degrading targeted protease fusions in terms of expression and activity, several constructs were made with differing proteases and fusion orientations to the anti-Aβ antibody, crenezumab, which is known to bind to Aβ monomers, oligomers, and fibrils with nanomolar affinity. Following purification using protein A resin, NEP fusions consistently showed the highest yield compared to the other proteases tested and were therefore chosen for further purification (FIG. 2C). For each NEP fusion format, protein A purified material was fractionated using size exclusion chromatography (SEC) (FIG. 2D), and fractions from each of the major peaks were tested for Aβ cleavage activity to identify the fraction(s) of interest (FIG. 2E).

As shown, IgG-CTF-1 and IgG-CTF-2 both dramatically enhanced potency on average 15-fold on Aβ(1-40) and 9-fold on Aβ(1-42) relative to their respective non-targeted controls (FIG. 3). Importantly, all Fc fusion controls showed comparable activity to unfused free enzyme, indicating that both N- and C-terminal fusion does not impair enzyme activity.

### Methods:

Molecular cloning: Gene fragments encoding all in-house derived constructs with human codon optimization were synthesized and cloned into the pRK mammalian expression vector. The pRK vector contains a cytomegalovirus (CMV) enhancer and promotor to control gene expression, an N-terminal secretion signal (MGWSCIILFLVATATGVHS; SEQ ID NO. 1), a C-terminal simian virus 40 (SV40) PolyA sequence, and an ampicillin resistance gene for bacterial selection. For Aβ protease constructs, NEP (Y52-W750), NEP2 (R74-W770), IDE (M42-L1019), or MTSP1 (G596-V855) were fused to either Fc (D221-K447, EU numbering) or full-length human IgG1 via a GGGGS (SEQ ID NO. 2) linker. For C-terminal protease fusions, the C-terminal lysine of the Fc was excluded. For all monovalent protease fusions, knob-in-hole mutations were introduced into the Fc to enable heterodimerization (Merchant et al. (1998) An efficient route to human bispecific IgG. Nat Biotechnol. 16, 677-681).

Protein expression and purification: Protein expression was performed by transfection of HEK293 cells with 30 μg DNA per 30 ml cell culture at a 1:1 heavy:light chain DNA ratio using standard protocols. Some formats only required transfection of a single DNA while others necessitated co-transfection of separate DNAs encoding, for example, heavy and light chains or knob and hole constructs. Affinity chromatography was carried out using MabSelect™ SuRe™ resin (Cytiva, 17543803) for Fc-containing proteins, CaptureSelect® CH1-XL resin (Thermo, 194346201L) for Fabs. For most antibody-enzyme fusion proteins, analytical size exclusion chromatography (SEC) revealed the presence of multiple species, likely representing a variety of cleavage products, mis-paired antibody subunits, and aggregates. To isolate the appropriate species within each sample, fractions were tested from each major peak within the SEC chromatogram for cleavage activity. Further SEC purification using a HiLoad 16/600 Superdex 200 column was required to isolate the desired monomeric species. Protein quality was determined by analytical SEC using a Waters xBridge BEH200A SEC 3.5 m (7.8×300 mm) column (Waters, 176003596) and by SDS-PAGE.

Aβ protease panel: NEP (R&D, 1182-ZNC-010), NEP2 (R&D, 2340-ZN-010), ECE-1 (R&D, 1784-ZN-010), ECE-2 (R&D, 1645-ZN-010), ACE (R&D, 929-ZN-010), IDE (R&D, 2496-ZN-010), MMP2 (R&D, 902-MP-010), MMP9 (R&D, 911-MP-010), MTSP1 (R&D, 3946-SEB-010) were purchased commercially for initial Aβ cleavage activity screening. Activation of a 100 μg/mL solution of MMP2 or MMP9 was performed by incubation in 1 μM APMA (Sigma, A9563) for 1 or 24 hours, respectively. Catalytic activity of each protease was confirmed using one of three control fluorogenic substrates: Mca-RPPGFSAFK(Dnp)-OH (R&D, ES005), Mca-PLGL-Dpa-AR-NH2 (R&D, ES001), or Boc-QAR-AMC (R&D, ES014). A 3-fold dilution series starting at 100 nM was constructed for each protease in Aβ assay buffer: 50 mM HEPES pH 7.4, 150 mM NaCl, and 0.05% Brij-35, a non-ionic surfactant known to enhance protein solubility (Sigma, B4184). Each dilution series was incubated with either 20 μM Mca-RPPGFSAFK(Dnp)-O, 60 μM Mca-PLGL-Dpa-AR-NH2, or 50 μM Boc-QAR-AMC in black 96-well plates (Corning, 3356) for 10 min. Fluorescence was measured on a Molecular Devices SpectraMax® M2 microplate reader with 320 nm excitation and 460 nm emission for Mca-RPPGFSAFK(Dnp)-O and Mca-PLGL-Dpa-AR-NH2 or 380 nm excitation and 460 nm emission for Boc-QAR-AMC.

Aβ cleavage assay: Aβ(1-40) (Anaspec, AS-24236) and Aβ(1-42) (Anaspec, AS-20276) substrates were resuspended in 1% ammonium hydroxide (Anaspec, AS-61322) to a concentration of 1 mg/mL. The solution was sonicated twice for 30 seconds on ice, aliquoted, and stored at −80° C. Prior to each assay Aβ was thawed on ice and a 200 nM working solution was made in Aβ assay buffer. A 3-fold dilution series of each protease or protease fusion was produced in Aβ assay buffer starting at 1.8 μM. 5 μL of protease dilution was added to 5 μL of Aβ and incubated at 37° C. for 1 hour. 10 μL of 20 μM 1,10-phanthroline (Sigma, 131377) was added to stop the reaction. Each sample was then diluted 10-fold using 180 μL of PBST (10 mM sodium phosphate pH 7.4, 150 mM NaCl, and 0.05% Tween® 20) and the concentration of intact Aβ was assessed as previously described (62). Biotinylated capture antibody anti-Aβ(1-16) clone 6E10 (Biolegend, 803009) was diluted to 1 μg/mL in PBST. Detection antibodies for Aβ(1-40) (in-house derived) and Aβ(1-42) (Thermo, 700254) were fluorescently labeled using an Alexa Fluor 647 antibody labeling kit (Thermo, A20186) and diluted to 25 nM in Rexxip™ F buffer (Gyros Protein Technologies, P0004825). A standard curve for Aβ(1-40) or Aβ(1-42) was constructed using a 3-fold, 12-point dilution series starting at 50 μM in PBST. All samples, including capture and detection antibodies, Aβ standards and diluted protease reactions, were loaded into 96-well plates (Thermo, AB0800) and run on a Gyrolab® xPand system using a 1000 nL CD (Gyros Protein Technologies, P0004253) and according to the manufacturer's standard protocol for 3-step ELISA with two wash buffers: PBST and pH 11 wash buffer (Gyros Protein Technologies, P0020096). The Gyros software was used to measure Aβ concentration by fitting to the standard curve.

### Example 2: Evaluation of the Catalytic Potency of Antibody-Enzyme Fusions

This Example investigated the impact of antigen affinity on anti-Ap antibody-enzyme activity, and demonstrated that when fused to an enzyme, antibodies facilitate rapid recycling of target antigen for cleavage by the fused protease.

Methods: Cleavage assay was performed as described in Example 1.

Aβ surface plasmon resonance: Solution affinity constants for anti-Aβ antibodies were assessed on a Biacore™ T200. Anti-Ap fusions were diluted to 1 μg/mL in HBS-P+ (Cytiva, BR100671) and captured using a Series S protein A chip (Cytiva, 29127555). A 3-fold, 8-point dilution series of Aβ(1-28) (New England Peptide, 22360) was constructed in HBS-P+ and injected for 5 min, followed by 5 min of dissociation. Affinity constants were obtained through kinetic fitting using the Biacore Evaluation Software.

### Results:

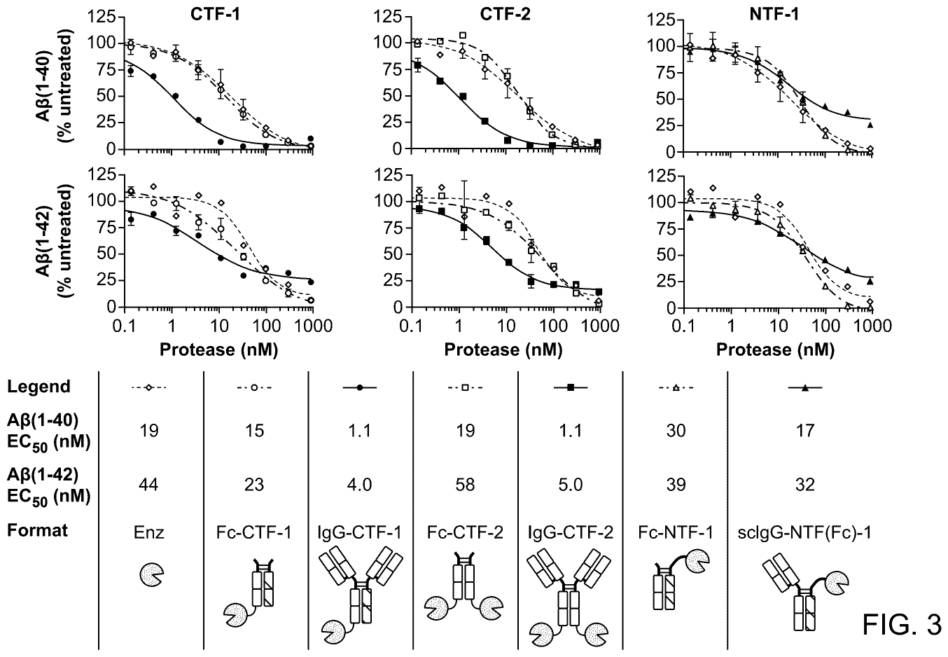

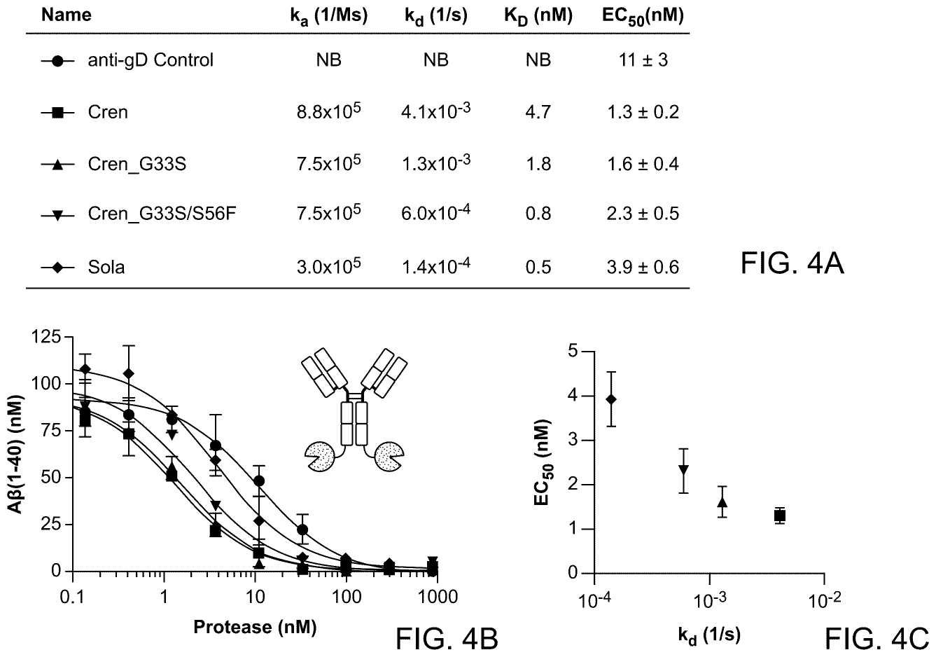

The anti-Aβ antibody, Crenezumab, which has both fast on and off rates (panel A in FIG. 4), was hypothesized to facilitate rapid recycling of target antigen for cleavage by the fused protease. To investigate the dependence of targeted catalysis on antibody binding kinetics and affinity, the bivalent NEP IgG-CTF-2 format with two variants of crenezumab with slower off-rates and, therefore, stronger affinities (G33S(HC) and G33S(HC)/S56F(LC)) was also constructed. A fusion of another anti-Aβ antibody, solanezumab, which shares high sequence identity to crenezumab (93% identity in VH and 92% identity in VL) and binds a similar epitope but with higher affinity was also constructed. Finally, a fusion containing a variable region targeting an unrelated antigen (gD) was produced as a non-binding control. Nucleotide sequences of the constructs are disclosed in Table 1.

TABLE 1
Neprolysin fusions

| Construct | SEQ ID NO: |
|---|---|
| IgG-CTF-2 format, neprolysin, anti-gD, heavy chain | 10 |
| IgG-CTF-2 format, neprolysin, anti-gD, light chain | 11 |
| IgG-CTF-2 format, neprolysin, crenezumab, heavy chain | 12 |
| IgG-CTF-2 format, neprolysin, crenezumab, light chain | 13 |
| IgG-CTF-2 format, neprolysin, solanezumab, heavy chain | 14 |
| IgG-CTF-2 format, neprolysin, solanezumab, light chain | 15 |

This resulted in a panel of five constructs of IgG-CTF-2 with varying off-rates and affinities yet similar on-rates (panel A in FIG. 4). Overall, higher Aβ cleavage activity correlated with faster off rate across the affinity variants with the crenezumab-NEP fusion (fastest off-rate) having a 3-fold lower EC50 than the solanezumab-NEP fusion (slowest off-rate) (panel B and C in FIG. 4). These data suggest that fast binding kinetics may be optimal, enabling antibody to cycle through Aβ binding events for more effective enzymatic turnover. Overall, the results suggest that faster off-rate and weaker Aβ affinity correlate with catalytic potency of enzyme fusions.

### Example 3: Protease Engineering for Immunoglobulin G Cleavage

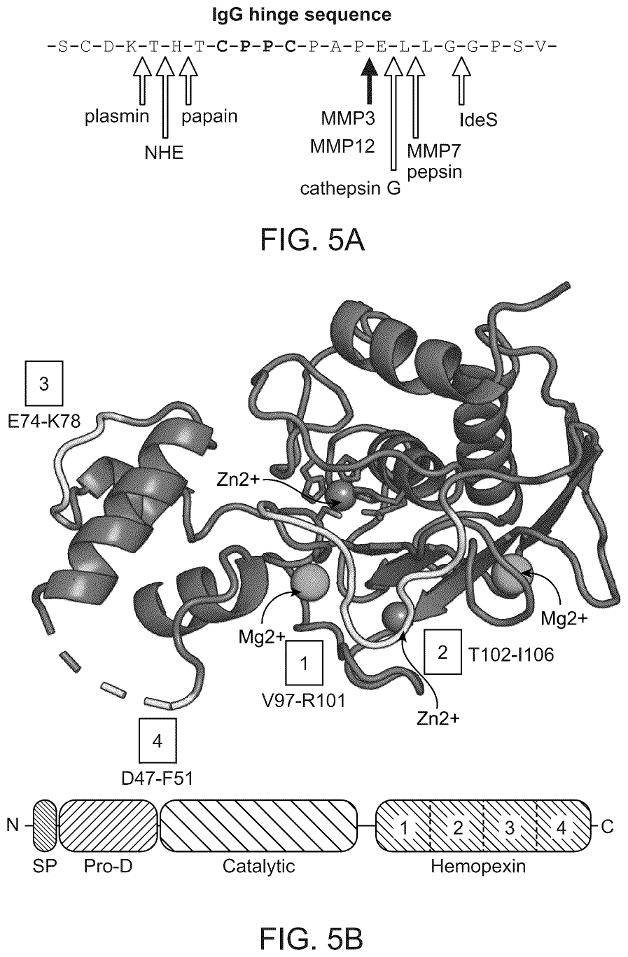

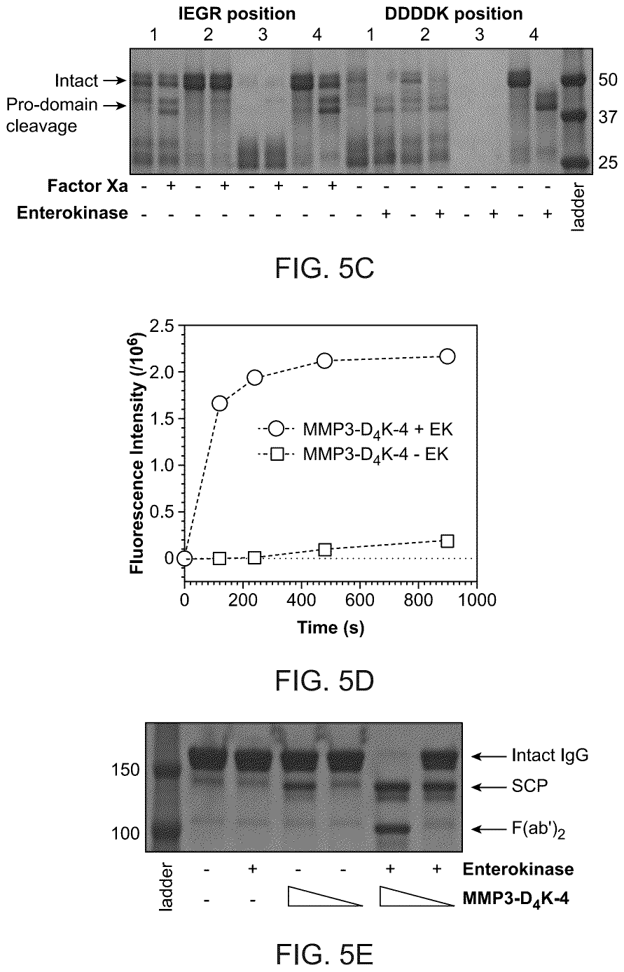

The applicability of the antibody-guided protease platform to turnover a target of high abundance, serum IgG was explored. Therapeutic IgG-suppression has been investigated for a wide range of autoimmune and inflammatory diseases with clinical success. Due to the extraordinarily high (˜10 mg/ml) concentration of IgG in human serum, catalytic turnover is essential. The IgG degrading enzyme IdeS is effective at depleting IgG in vitro and in vivo and has been tested clinically. However, due to its bacterial origin IdeS is highly immunogenic, hindering repeat dosing and, as a consequence, its therapeutic application is limited to acute treatment indications such as kidney transplantation. Several human enzymes with lower immunogenicity risk have been shown to cleave IgG (FIG. 5A), but they are not IgG-selective and lack the efficiency needed to clear the high levels of substrate present in serum. Targeted catalysis was investigated as a means to enhance the cleavage efficiency and selectivity of human proteases to degrade serum IgG.

### Methods:

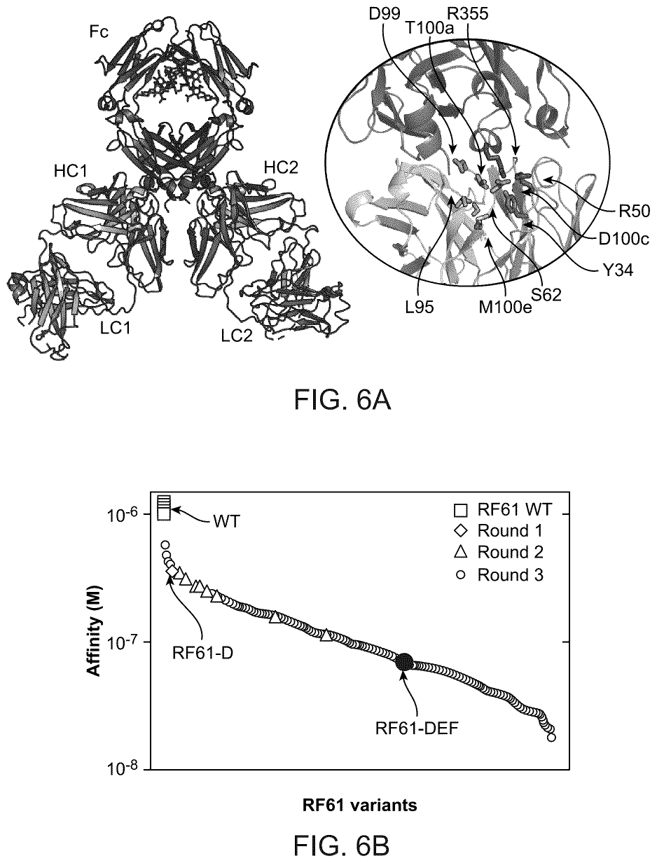

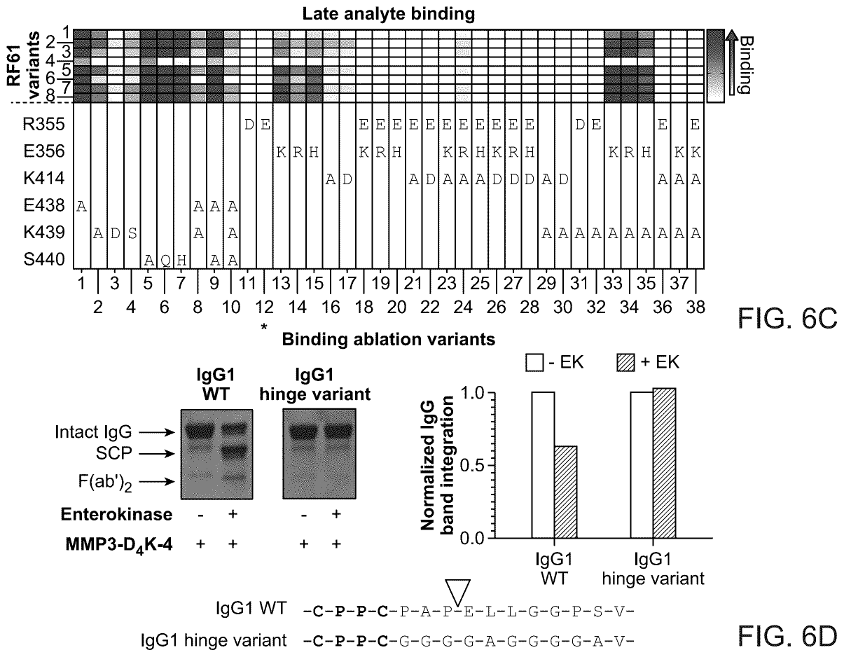

Molecular cloning: Gene fragments encoding all in-house derived constructs with human codon optimization were synthesized and cloned as described in Example 1. For IgG protease constructs, MMP3 (Y18-C477) was fused to the N-terminus of either Fc (D221-K447, EU numbering), full-length human IgG1 heavy chain, Fab heavy chain (Q1-S113, Kabat numbering), or full-length human kappa light chain via a (GGGGA)₂ (SEQ ID NO. 4) linker. The gene for MMP3 only (Y18-C477) was synthesized with a C-terminal FLAG-tag for purification, and the enterokinase (DDDDK; SEQ ID NO. 5) and factor Xa (IEGR; SEQ ID NO. 6) protease cleavage sites were inserted via site-directed mutagenesis using standard protocols (Qiagen®, 210513). Genes introducing Fc mutations for RF61 binding ablation (FIG. 6C) and an alternate hinge sequence for MMP3 resistance (FIG. 6D) were synthesized within a human IgG1 framework (Wuxi).

Protein expression and purification was performed as described in Example 1.

### Results:

Three human proteases (matrix metalloproteinase 3 (MMP3), MMP7, and Cathepsin G) were selected based on their human origin and IgG cleavage properties and screened for expression. For further development MMP3 was selected. MMP3 is a zinc matrix metalloproteinase expressed by a broad variety of cell types with promiscuous activity against matrix and bioactive substrates. The structure consists of an N-terminal signaling sequence, pro-domain, catalytic domain, and hemopexin domain connected by a proline-rich linker (FIG. 5B). Successful expression of MMP3 required fusion to the inhibitory N-terminal pro-domain, which was later proteolytically removed to induce activity. While various approaches, including heat, addition of organo-mercury reagents, or partial proteolysis, have been reported to induce activation in vitro, these strategies also had significant risks to the structure and stability of a potential therapeutic.

To circumvent the problematic activation step (removal of inhibitory N-terminal pro-domain), MMP3 variants capable of pro-domain cleavage by the highly specific proteases enterokinase (EK) and Factor Xa (Xa) were designed. In order to determine the optimal cleavage location to achieve full and selective MMP3 activity, we substituted the recognition sequences for EK or Xa within the pro-domain of MMP3 at four unstructured locations to both allow for maximal EK or Xa protease accessibility and to minimize structural perturbation of the pro-domain (FIG. 5B). SDS-PAGE analysis and MMP3 activity assays were performed on the eight variants with and without the addition of EK or Xa. The variant with the EK site insertion at location 4 showed the best combination of high stability with low catalytic activity in the native state (with pro-domain) while yielding efficient removal of the pro-domain in the presence of EK (FIGS. 5C and 5D). Therefore, the original variant with the EK site inserted at position 4, referred to as MMP3-D4K-4, was selected for further studies. At high concentrations and after EK cleavage of the pro-domain, MMP3-D4K-4 cleaved the IgG hinge (FIG. 5E).

### Example 4: Evaluation of the Catalytic Potency of Anti-IgG Antibody-Enzyme Fusions

In this Example, an antibody that weakly recognizes IgG was affinity matured, then fused to a protease. The antibody-enzyme fusion was then tested for targeted IgG cleavage capabilities.

### Example 4A: Engineering of RF61 to Increase Binding Affinity to IgG

Engineering a non-self selective anti-IgG antibody for endogenous IgG targeting. To target MMP3 to IgG, the use of Rheumatoid factors (RFs) was explored, which are naturally occurring human autoantibodies that bind to IgG. A well-characterized RF, referred to as RF61, which was first isolated from a rheumatoid arthritis patient and binds to the antibody Fc region was used (Harindranath, N. et al. (1991) Complete sequence of the genes encoding the V H and V L regions of low- and high-affinity monoclonal 1 gM and lgA1 rheumatoid factors produced by CD5+B cells from a rheumatoid arthritis patient. Int Immunol. 3, 865-875). Importantly, the crystal structure of RF61 in complex with Fc has been solved (Duquerroy et al. (2007) Crystal Structure of a Human Autoimmune Complex between IgM Rheumatoid Factor RF61 and IgG1 Fc Reveals a Novel Epitope and Evidence for Affinity Maturation. J Mol Biol. 368, 1321-1331) and suggested the possibility for non-self selectivity engineering (below).

### Methods:

RF61 affinity maturation: Affinity maturation of RF61 was performed by mutating each residue within the heavy and light chain CDRs to the other 18 possible residues (excluding cysteine) with a 2-step PCR protocol using PrimeSTAR® Max DNA polymerase (Takara, R045B) according to standard protocols, generating 18 single point mutants per CDR residue. For rounds one and two, the heavy chain template DNA contained only the VH and CH1 domains to produce recombinant Fab proteins. Fab protein variants were expressed via co-transfection of heavy and light chain DNAs at 1 mL scale in HEK293 cells and purified with CaptureSelect® CH1-XL resin for affinity screening. For round three, RF61 heavy and light chain variable domains were fused to mouse IgG2a constant domains to create chimeric full-length antibodies that do not bind to their own Fc domains, as RF61 does not bind to mouse IgG2a. Protein expression was performed as described above followed by purification using MabSelect™ SuRe™ resin.

RF61 surface plasmon resonance: RF61 affinity for human IgG was assessed with a Biacore 8K+ or T200. For rounds one and two of affinity maturation, the Fc domain of human IgG1 was captured on a Series S Protein A chip according to the manufacturer's protocols. Serial dilutions of the RF61 Fab variants were prepared in HBS-P+ buffer. The dilutions were passed over the chip for 4 min, followed by a 5 min dissociation step. Variants were assessed using the response units at the point of late analyte binding normalized to Fc capture level. For round three of affinity maturation, RF61 chimeric antibody variants were captured on a Series S CM5 chip (Cytiva, 29104988) containing immobilized anti-mouse antibodies from a mouse antibody capture kit (Cytiva, 29215281). Serial dilutions of human IgG1 Fc in HBS-P+ were passed over the chip for 10 minutes, followed by a 6 min dissociation step. Affinity constants were obtained through kinetic fitting using the Biacore Evaluation Software (GE). To evaluate the Fc mutations for RF61 binding ablation, a selection of eight RF61 chimeric antibody variants spanning a range of binding strengths from round three of affinity maturation (R50D LC; R50N LC, L95N D99H T100aA HC; R50N LC, S62P L95N D99H T100aA D100cE M100eF HC; R50N Y34F LC, S62P D99H D100cE M100eF HC; R50D LC, S62P L95N T100aA D100cE M100eF HC; R50D Y34F LC, D100cE HC; R50D Y34F LC, S62P D99H M100eF HC; R50D Y34F LC, S62P L95N D100cE M100eF HC) were captured as described above. A single 1000 nM concentration of each binding ablation variant was passed over the chip for 10 minutes, followed by a 6 min dissociation step. The late analyte binding signal (RU) was normalized to antibody capture level to quantify binding, and the signal from the eight RF61 variants is presented in FIG. 6C. To characterize affinity of the antibody-MMP3 fusion constructs, the IgG1 subtype of anti-HER2 antibody, 4D5, was captured on a Series S Protein L chip (Cytiva, 29205138) according to the manufacturer's protocols. Serial dilutions of the RF61-MMP3 fusion constructs were prepared in HBS-P+ buffer. The dilutions were flowed over the chip for 3 min, followed by 8 min of dissociation. RF61 contains a lambda light chain, so it does not bind to the Protein L chip. Affinity constants were determined as described above.

### Results:

RF61 was initially identified as an IgM, which binds weakly to IgG₁ Fc with a KD of approximately 600 nM (including avidity). The crystal structure shows a stoichiometry of two RF61 Fabs per Fc with each Fab contacting residues from both CH3 domains (FIG. 6A). To improve the affinity of RF61 for the IgG Fc, saturation mutagenesis (excluding cysteine) at each residue of the complementarity-determining regions (CDRs) of both the heavy chain (HC, 38 residues) and light chain (LC, 31 residues) was performed, totaling 1242 single RF61 point mutants. While binding of wildtype RF61 Fab to Fc in a monovalent surface plasmon resonance (SPR) binding format was not able to be detected, screening of mutants using the same technique identified four promising mutations with significantly increased affinity all located in the LC (R50N, R50D, Y34F, and Y34N). A second round of screening was performed in which the R50N mutant LC was paired with the same HC single point mutant library (684 total variants). SPR screening revealed six HC mutations with improved binding upon combination with the R50N LC (S62P, L95N, D99H, T100aA, D100cE, and M100eF). For the third and final round, 320 variants with combinations of the selected LC and HC mutations were produced, ranging from one to eight mutations per variant. The three rounds of our saturation mutagenesis screen yielded RF61 variants spanning over 2 logs in affinity from >1 μM to ˜10 nM (FIG. 6B).

Additional engineering of the RF61 IgG format was needed for an effective endogenous IgG targeting antibody. First, the Fc needed to be modified to avoid self-recognition. Using the crystal structure of RF61 bound to an IgG1 Fc as a guide, Fc variants containing between one and four mutations were designed to ablate RF61 binding. SPR was used to screen the Fc variants against a panel of eight affinity-improved RF61 variants (FIG. 6C). The single mutant R355E showed exquisite ablation of RF61 binding, with minimal improvement from additional mutations, and was therefore selected. A second requisite is that the hinge must be resistant to proteolysis by MMP3 to avoid self-cleavage by the fused enzyme. We replaced the ten residues following the hinge disulfides, effectively the lower hinge and N-terminal region of the CH2 domain, with a (G4A)2 linker to confer resistance to MMP3 cleavage (FIG. 6D). Altogether, the IgG-targeting antibody contains Fab arms with improved RF61 affinity, an Fc with the R355E mutation to avoid self-binding, and a mutated lower hinge to avoid self-cleavage by MMP3.

### Example 4B: Fusion Formats and Activities

### Methods:

MMP3 activity assay: All MMP3-D4K-4 and MMP3-D4K-4 fusion protein samples were exchanged into cleavage buffer (10 mM HEPES, 150 mM NaCl, and 10 mM CaCl₂ at pH 7.5). MMP3-D4K-4 was activated with 16 units of enterokinase (NEB, P8070L) for every 25 μg protein through incubation at room temperature for 16 hours. To inactivate the enterokinase, 0.1 mg/ml soybean trypsin inhibitor (Sigma, 17075029) was added to the protein solution. 50 μL of 2.5 μM fluorogenic MMP3 peptide substrate (R&D Systems, ES002) or 50 μg/ml DQ-collagen-IV (Invitrogen, D12052) in cleavage buffer was combined with the desired concentration of activated MMP3-D4K-4 sample within a 96-well black flat-bottom plate (Corning, CLS3925), and the fluorescence signal was measured on a Molecular Devices SpectraMax® M2 microplate reader (Molecular Devices) with 320/405 nm and 485/535 nm excitation/emission for the peptide substrate and DQ-collagen-IV, respectively. Concentration-dependent assays were performed in duplicate with 2.5-fold dilutions from 400 nM.

IgG cleavage assay: MMP3-D4K-4 fusion proteins were activated as described above with enterokinase. Seven 2.5-fold dilutions of the activated fusion proteins were prepared in cleavage buffer starting at 800 nM. 4 μL of each dilution was mixed with 4 μL of the antibody substrate (one-arm anti-gD IgG1 antibody at 800 nM). The cleavage reaction was incubated at 37° C. for 24 hours. The extent of antibody cleavage was assessed via ELISA as follows. 100 μL of Affinipure goat anti-human Fc antibody (Jackson ImmunoResearch, 109-005-098) at 1.2 μg/mL was added to each well of a Maxisorp™ 96 well plate (Thermo, 44-2404-21). The plate was incubated at room temperature for 1 hour, then washed 3 times with PBST. The wells were blocked with SuperBlock™ buffer (Thermo, 37515) for 1 hour at room temperature followed by 3 washes with PBST. Each cleavage reaction was diluted to 100 ng/mL of the one-arm anti-gD substrate in PBST (400-fold), then 100 μL of the dilutions were added to the blocked wells. The plate was incubated at room temperature for 1 hour, then washed 5 times with PBST. 100 μL of a 1:40,000-fold dilution of a goat anti-human Fab HRP-conjugated antibody (Sigma, A0293) was added to each well and incubated for 1 hour at room temperature. The wells were washed 5 times with PBST. 100 μL of TMB substrate (Thermo, N301) was added to each well, and the reaction proceeded for 15 min at room temperature before quenching with the stop solution (Thermo, N600). Absorbance was measured at 405 nm.

IgG and fluorogenic peptide substrate cleavage assays in human serum: MMP3-D4K-4 Fab fusion proteins were activated as described above with enterokinase. Soybean trypsin inhibitor was not added to the fusion proteins in order to observe any effects from endogenous protease inhibitors in the serum. Eight 2-fold dilutions of the fusion proteins starting at 600 nM were prepared in the cleavage buffer described above. The serum was prepared from a blood sample of a single human donor through centrifugation and collection of the supernatant. Before use, the serum was diluted 1:1 in cleavage buffer. 25 μL of serum was combined with 25 μL of the antibody dilution, resulting in a top fusion concentration of 300 nM. The cleavage reaction was incubated at 37° C. for 24 hours. IgG₁ cleavage was detected with the ELISA assay described above with two important modifications. First, the antibody used to coat the Maxisorp 96 well plate was a mouse anti-human IgG1 antibody at 1.0 μg/mL in PBS (Thermo, MH1015). This modification ensured that the ELISA would detect cleavage of IgG₁ only rather than all subtypes of IgG in the serum. Second, the cleavage reactions were diluted 100-fold in PBST before addition to the Maxisorp plate. The optimal dilution factor was determined with a by performing the ELISA assay with a dilution series of the serum. The rest of the assay was performed as described above. For the fluorogenic peptide substrate cleavage assay, 25 μL of the cleavage reaction in serum was combined with 25 μL of the fluorogenic MMP3 peptide substrate (R&D Systems, ES002) at 5 μM in cleavage buffer, and the sample was added to wells of a 384-well black flat, clear bottom plate (Thermo, 242764). The final top protease fusion concentration for the peptide cleavage assay was 150 nM with seven additional 2-fold dilutions. The plate was sealed and incubated at room temperature for one hour before reading the fluorescence signal with a Perkin-Elmer EnVision® plate reader.

### Results:

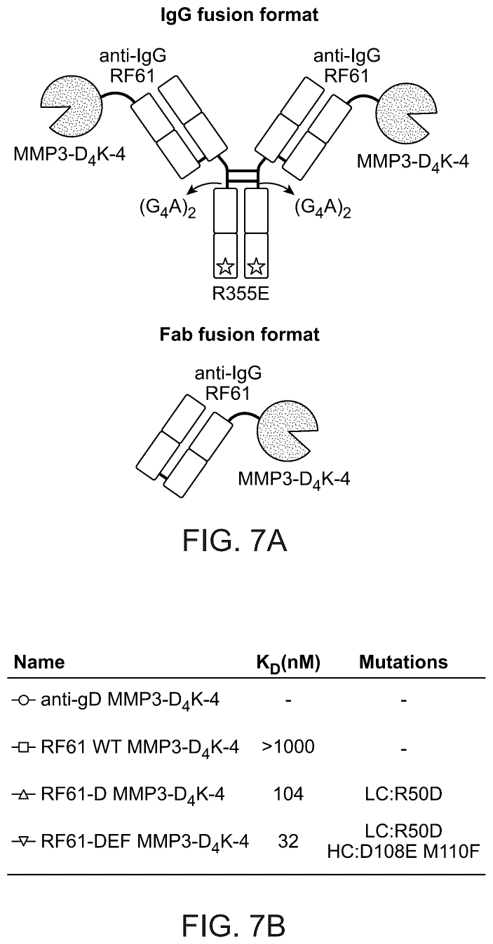

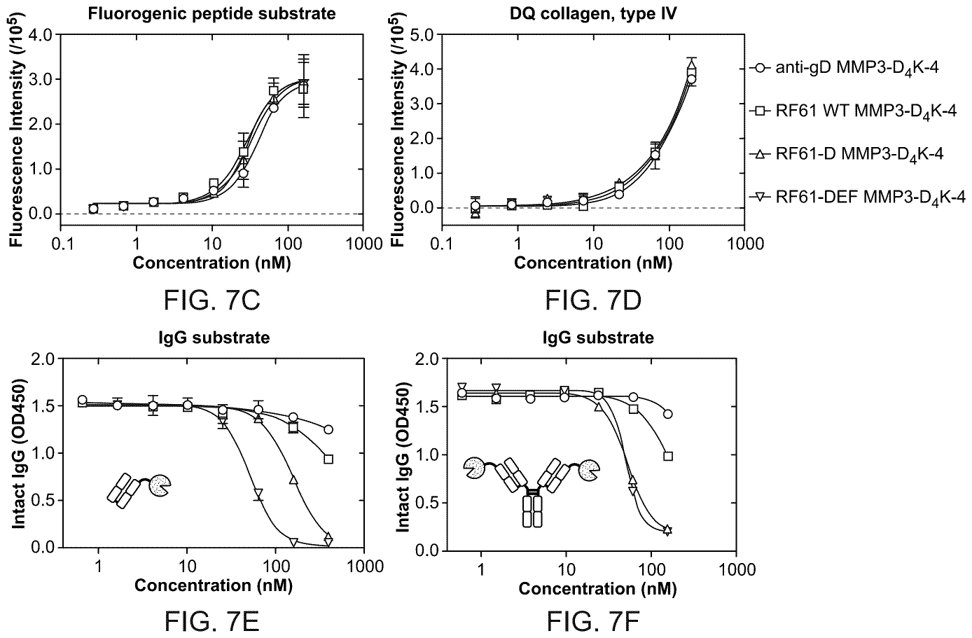

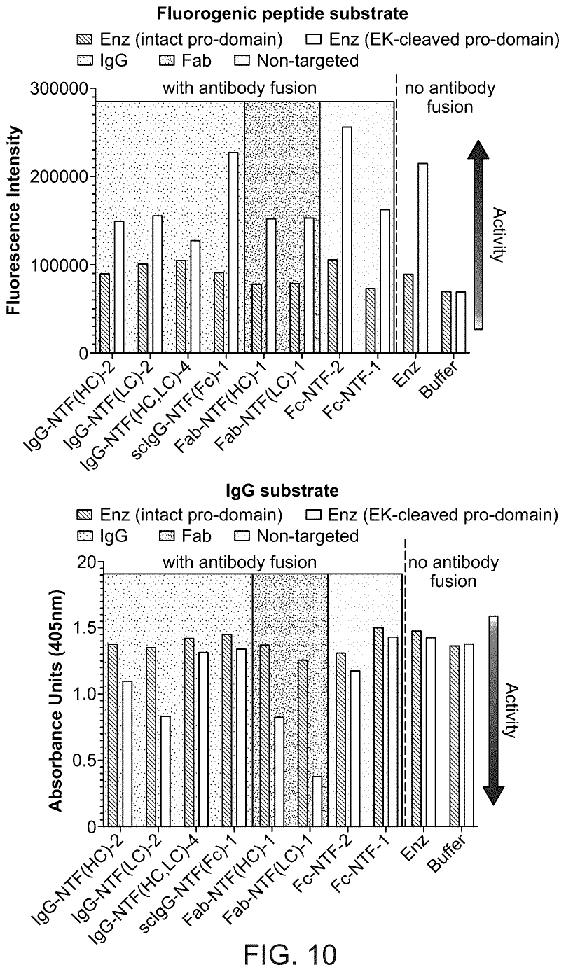

Two engineered modalities as described above were combined into a single targeted protease. In a similar approach as Aβ, a variety of antibody-enzyme fusion formats were screened for expression, stability, affinity, and activity. Since C-terminal fusions of MMP3 to the antibody would result in separation of the proteolytic and targeting modalities upon the pro-domain cleavage needed to activate MMP3, efforts were focused on the N-terminal fusions. Fusion of MMP3 and its pro-domain to the N-termini of the antibody HC, LC, or Fc yielded sufficient purified material. Activity screening against both fluorogenic peptide substrate and IgG substrate revealed the most promising IgG format as an MMP3 fusion to the LC N-terminus via a (G₄A)₂ linker, referred to as IgG-NTF(LC)-2 (FIGS. 7A and 10). Both IgG and Fab formats of the MMP3 LC N-terminal fusion were scaled up and purified for further study (data not shown).

In order to determine whether RF61-mediated IgG targeting of MMP3 could enhance its activity, three antibody-MMP3 fusion proteins with varying affinity towards endogenous human IgG were designed (protease fusions to the N-terminus of the light chain as described in Table 2; FIG. 7A, bottom panel).

TABLE 2
IgG-NTF(LC)-2 format MMP3 fusion nucleotide sequences

| | SEQ |
|---|---|
| | ID |
| Description | NO: |
| IgG-NTF(LC)-2 format, MMP3-D4K-4, anti-gD, heavy chain | 16 |
| IgG-NTF(LC)-2 format, MMP3-D4K-4, anti-gD, light chain | 17 |
| IgG-NTF(LC)-2 format, MMP3-D4K-4, RF61 wild-type, heavy | 18 |
| chain | |
| IgG-NTF(LC)-2 format, MMP3-D4K-4, RF61 wild-type, light | 19 |
| chain | |
| Fab-NTF(LC)-1 format, MMP3-D4K-4, RF61 wild-type, heavy | 20 |
| chain | |
| Fab-NTF(LC)-1 format, MMP3-D4K-4, RF61 wild-type, light | 21 |
| chain | |

Although binding of wildtype RF61 to Fc was not detectable SPR, it was still included based on previous reports that it was a weak binder to IgG. Two RF61 mutants from the saturation mutagenesis screen described above were also included: RF61-D (104 nM), and RF61-DEF (32 nM) (FIG. 7B). A gD control format was also produced as a true non-binding control. Each affinity variant and control were produced with MMP3 fused to the LC of both a Fab and full-length IgG. Monovalent affinities for each targeting arm are summarized in FIG. 7B. Non-targeted activity was first measured against two non-IgG substrates: a fluorogenic peptide substrate, and DQ collagen IV that represents an endogenous off-target substrate (FIG. 7C). As expected, the activity curves for each member of the affinity series overlap well, signifying non-selectivity of MMP3 for non-IgG substrate. To determine the effect of IgG targeting, Fab fusion and IgG fusion proteins were incubated at various concentrations with a human IgG1 substrate, and IgG cleavage was determined with an ELISA-based assay (FIGS. 7C-7F). EC50 values correlated remarkably well with IgG affinity of the targeting arm, with the Fab fusion but not IgG fusion format differentiating between the two higher affinity RF61 variants (FIG. 7C). The non-targeted anti-gD construct showed little to no cleavage at the maximum tested concentration. While a quantitative measure of enhancement over the non-targeted construct could not be determined due to the lack of IgG cleavage from the anti-gD control, the results suggest that antibody targeting provides at least one log enhancement of protease activity.

Interestingly, while the EC50 values match for the Fab and IgG fusion formats of the RF61-DEF affinity variant (FIG. 7C), the RF61-D IgG fusion had an EC50 3-fold lower than that of the Fab fusion. These data suggest that avidity in the IgG fusion may contribute to enhanced activity. Furthermore, the lack of enhanced activity upon increased affinity for the two tightest binding IgG fusion formats indicates that a maximum beneficial effect of IgG-targeting was achieved, and the activity of the MMP3-D4K-4 protease is the limiting factor.

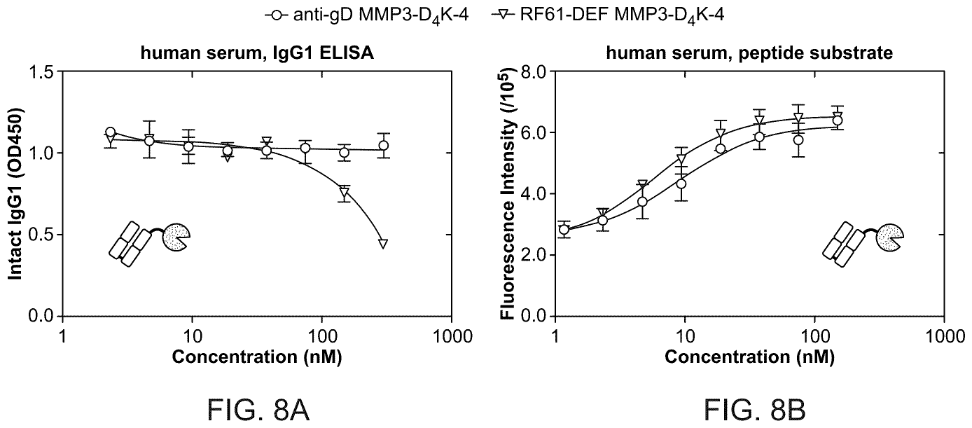

To assess the activity of the RF61-mediated IgG targeting of MMP3 in a more biologically relevant environment, the cleavage of IgG1 within human serum from both a targeted (RF61-DEF) and non-targeted (anti-gD) Fab fusion format was measured (panel A in FIG. 8). While the overall activity (EC50) of the targeted protease is reduced compared to the results against IgG1 in vitro, there still exists a clear activity enhancement compared to the non-targeted control. The reduced activity could be due to a variety of factors, including presence of endogenous protease inhibitors and greater levels of additional MMP3 substrates (e.g., many matrix and bioactive substrates including other immunoglobulin subtypes) and/or RF61 antigens (e.g., IgG₂ and IgG₃) in human serum compared to IgG1-containing buffer. Cleavage activity against a fluorogenic MMP3 peptide substrate suggests that endogenous protease inhibitors within human serum do not impact the activity of these fusion constructs (panel B in FIG. 8). The promising results described here suggest that there may be an accessible therapeutic window for IgG degradation in vivo.

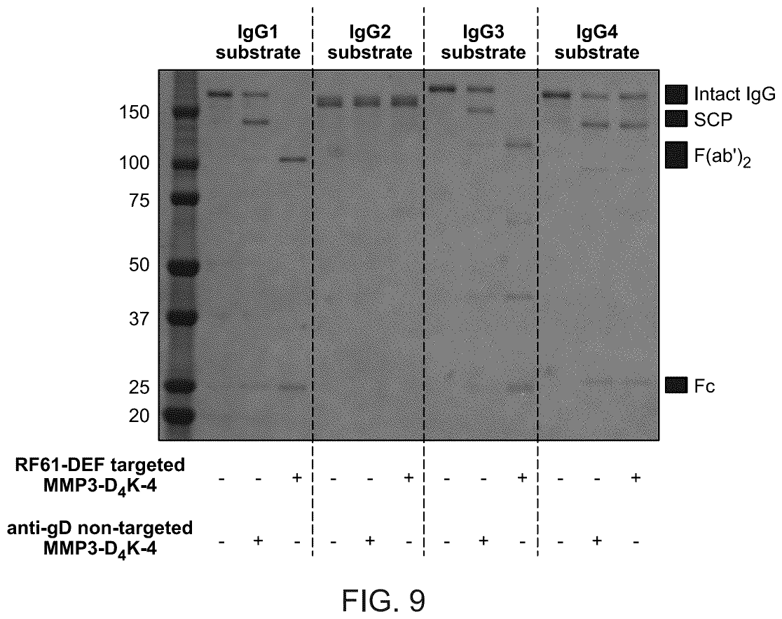

Although IgG₁ accounts for approximately two thirds of all human IgG, there are three other human IgG subclasses present in serum: IgG₂, IgG₃, and IgG₄. To assess the subtype dependence of our targeted protease, the ability of targeted (RF61-DEF) and non-targeted (anti-gD) antibody-MMP3 fusions to cleave all four human IgG subtypes was measured (FIG. 9). IgG₁ and IgG3 substrates showed similar enhanced targeting-dependent cleavage, with the RF61-DEF fusion fully cleaving IgG into Fc and F(ab′)2 fragments. In contrast, anti-gD control fusions were only capable of single hinge cleavage events for the same two subtypes. No cleavage of IgG2 was observed, which is consistent with the lack of an MMP3 cleavage site in its hinge sequence together with previous reports. No difference between targeted and non-targeted cleavage was observed for the IgG₄ substrate, which was anticipated based on the lack of RF61 binding to IgG₄. (Data not shown.) Altogether the results are consistent with the selectivity enhancement provided by targeted catalysis.

1. A fusion protein comprising an antibody that binds to a target, wherein the antibody is fused to a protease that cleaves a substrate, wherein the target is in the proximity of the substrate.

2-3. (canceled)

4. The fusion protein of claim 1, wherein the antibody comprises:
a) a heavy chain variable domain (VH) and a light chain variable domain (VL), wherein the protease is fused to the N-terminus or C-terminus of the VH or VL; or
b) a full-length antibody comprising two heavy chains and two light chains, wherein the protease is fused to the N-terminus or the C-terminus of one or both of the two heavy chains and/or the two light chains.

5-12. (canceled)

13. The fusion protein of claim 1 or 4, wherein the protease is fused with the antibody via a linker.

14-35. (canceled)

36. An isolated nucleic acid encoding the fusion protein of claim 1, 4 or 13 or a fragment thereof.

37. A host cell comprising the nucleic acid of claim 36.

38. A method of producing the fusion protein of claim 1, 4 or 13 or a fragment thereof comprising culturing the host cell of claim 37 under conditions suitable for the expression of the fusion protein or a fragment thereof.

39-40. (canceled)

41. A pharmaceutical composition comprising the fusion protein of claim 1, 4 or 13 and a pharmaceutically capable carrier.

42-44. (canceled)

45. A method of treating an individual having a disease or condition comprising administering to the individual an effective amount of the fusion protein of claim 1, 4 or 13 or the pharmaceutical composition of claim 41.
